SEQUENCE LISTING (7 sequences)

SEQ ID NO: 1 - protein, 6 aa, Artificial Sequence
  Synthetic sequence
PLGLAG

SEQ ID NO: 2 - protein, 4 aa, Artificial Sequence
  Synthetic sequence
RGDR

SEQ ID NO: 3 - protein, 9 aa, Artificial Sequence
  Synthetic sequence
CRGDKGPDC

SEQ ID NO: 4 - protein, 6 aa, Artificial Sequence
  Synthetic Sequence
  MOD_RES (3)..(4): Any amino acid
RKXXRK

SEQ ID NO: 5 - protein, 9 aa, Artificial Sequence
  Synthetic sequence
SPSYVYHQF

SEQ ID NO: 6 - protein, 5 aa, Artificial Sequence
  synthetic sequence
PLGVR

SEQ ID NO: 7 - protein, 8 aa, Artificial Sequence
  synthetic sequence
GPLGIAGQ

(19) United States
(12) Patent Application Publication
(10) Pub. No.: US 2023/0218771 A1
(43) Pub. Date: Jul. 13, 2023

(54) SELF-ASSEMBLING PRODRUGS AS IMMUNE BOOSTERS FOR CANCER IMMUNOTHERAPY

(71) Applicant: THE JOHNS HOPKINS UNIVERSITY, Baltimore, MD (US)

(72) Inventors: Honggang Cui, Lutherville, MD (US); Feihu Wang, Baltimore, MD (US); Hao Su, Baltimore, MD (US)

(21) Appl. No.: 17/759,382

(22) PCT Filed: Feb. 4, 2021
(86) PCT No.: PCT/US2021/016597

§ 371 Date: Jul. 25, 2022

Related U.S. Application Data
(60) Provisional application No. 62/969,883, filed on Feb. 4, 2020.

(51) Int. Cl. A61K 47/64; A61K 39/395; A61K 47/65; A61K 9/06; A61P 35/00; A61K 31/7084

(52) U.S. Cl. CPC A61K 47/64; A61K 39/39541; A61K 47/65; A61K 9/06; A61P 35/00; A61K 31/7084

(57) ABSTRACT

The disclosure is directed to compositions comprising a prodrug and an immunomodulator, which can self-assemble into a nanofiber hydrogel at the site of application in a human. The prodrug comprises one or more cytotoxic agents conjugated to a hydrophilic moiety by a linker. The compositions may be used to kill cancer cells, such as glioblastoma and colorectal cancer cells.

### STATEMENT OF RELATED APPLICATIONS

This application is a national phase application under 35 U.S.C. § 371 of PCT International Application No. PCT/US2021/016597, filed Feb. 4, 2021, which claims priority to U.S. Provisional Patent Application No. 62/969,883, filed on Feb. 4, 2020, which is hereby incorporated by reference for all purposes as if fully set forth herein.

### INCORPORATION-BY-REFERENCE OF MATERIAL SUBMITTED ELECTRONICALLY

The text of the computer readable sequence listing filed herewith, titled “JHU-39025-252_SQL”, created Jul. 25, 2022, having a file size of 12,563 bytes, is hereby incorporated by reference in its entirety.

### BACKGROUND OF THE INVENTION

Cancer immunotherapy has shown great promise in treating patients with advanced or metastatic tumors. However, only patients with immune-stimulating tumors, which are characterized by favorable tumor-infiltrating lymphocytes (TILs), seem to achieve effective clinical responses. The tumor microenvironment (TME) is the primary location where tumor cells and the TILs interact; the responsiveness of tumors to immunotherapy therefore depends, at least in part, on the TME immunophenotype. As a consequence, promoting an immune-stimulating TME that favors TILs has been proposed to be one of the most critical methods for maximizing the potential of cancer immunotherapy.

Thus, there remains a need for compositions and methods that not only kill cancer cells, but also stimulate and maintain an anti-cancer immune response in the TME.

### BRIEF SUMMARY OF THE INVENTION

The present disclosure provides a composition comprising: (a) a prodrug comprising one or more cytotoxic agents conjugated to a hydrophilic moiety by a linker, and (b) one or more immunomodulators. Also provided is a hydrogel comprising the composition and methods of using the composition to kill cancer cells.

### BRIEF DESCRIPTION OF THE DRAWING(S)

FIG. 1 shows a schematic and characterization of an in situ formed self-assembling prodrug hydrogel (P-NT-aPD1). (1A) Schematic illustration of localized CPT and aPD1 delivery using an in situ formed supramolecular hydrogel to attain bio-responsive drug release and tumor microenvironment regulation. (1B) Representative TEM images of diCPT-PLGLAG-iRGD nanotubes (P-NT). Scale bar: 100 nm. (1C) The circular dichroism (CD) spectrum of the diCPT-PLGLAG-iRGD nanotubes solution. (1D) Photographs of the solution to hydrogel (sol-gel) transition of P-NT upon the addition of PBS. (1E) Degradation profiles of diCPT-PLGLAG-iRGD in the presence or absence of GSH (10 mM). Data are given as mean±SD (n=3). (1F) In vitro cytotoxicity studies of free CPT and diCPT-PLGLAG-iRGD towards GL-261 brain cancer cells. (1G) Inhibition of tumor spheroid growth was evaluated following treatment with free CPT or P-NT. Spheroids treated with drug-free DMEM were used as the blank control. (1H) The degradation profiles of 200 μM diCPT-PLGLAG-iRGD solutions incubated in the presence or absence of MMP-2 (2 μg/ml). Data are given as mean±SD (n=3). (1I) Cumulative release profiles of CPT prodrugs (including diCPT-PLGLAG-iRGD and diCPT-PLG) and (1J) aPD1 from P-NT-aPD1 hydrogels incubated in PBS with or without MMP-2. Data are given as mean±SD (n=3).

FIG. 2 shows that a P-NT-aPD1 hydrogel embodiment enhances local retention and prolongs in vivo release of aPD1. (2A) In vivo gel formation and retention after subcutaneous injection of P-NT solution in the back of C57BL/6 mice. (2B) In vivo degradation profile of the P-NT hydrogel over time, as determined by the mass loss method. (2C) Fluorescence IVIS imaging of the local retention and distribution of aPD1-Cy5.5 in mice, administered in solution form and with the P-NT hydrogel. Experiments were repeated three times. (2D) Fluorescence imaging of tumor tissues and (2E) tumor sections of GL-261 brain tumor-bearing mice after tumoral injections of free (CPT+aPD1) or P-NT-aPD1. Red: Cy5.5 labelled aPD1. Blue: DAPI stained nuclei. Scale bar: 200 μm. Quantification of the in vivo retention profile of Cy5.5-aPD1 (2F) and (2G) CPT. Statistical significance was calculated using a two-sided unpaired t-test. Data are given as mean±SD (n=3). *P≤0.05, **P≤0.01.

FIG. 3 depicts local delivery of aPD1 by a CPT prodrug hydrogel embodiment elicits a robust antitumor immunity. (3A) Experimental schedule: GL-261 brain cancer cells were implanted into the right flank of mice on day 0. Mice were intratumorally (it.) injected on day 10 with P-NT, aPD1 loaded diC₁₂-PLGLAG-iRGD (aPD1-L), or P-NT-aPD1 hydrogels, with diC₁₂-PLGLAG-iRGD hydrogel (E-gel) used as a drug free control. Flow cytometric analysis was performed on lymphocytes extracted from the tumor on day 25. (3B) Representative flow cytometric analysis images and (3D) relative quantification of CD3⁺ T cells. (3C) Representative flow cytometric images and (3F) relative quantification of CD8⁺ T cell infiltration within the tumor by different treatment groups. (3E) Quantification of CD4⁺ T cell that infiltrated within the tumor in different treatment groups. (3G) Representative flow cytometric analysis images and (H) relative quantification of Foxp3⁺CD4⁺ T cells (T_{regs}). (3I) The percentages of PD-1⁺ CD8⁺ T cells and (J) PD-1⁺ CD4⁺ T cells within the tumor tissues after different treatments. (3K) The percentage of PD-L1 expressing tumor cells after different treatments. Statistical significance was calculated using a two-sided unpaired t-test. Data are given as mean±SD (n=3). *P≤0.05, **P≤0.01, ***P≤0.001.

FIG. 4 depicts intra-tumoral injection of P-NT-aPD1 prodrug hydrogel elicits complete regression of established GL-261 brain tumors. (4A) Experimental schedule: GL-261 brain cancer cells were implanted into the right flanks of mice on day 0. Mice were intratumorally (it.) injected on day 10 with free (CPT+aPD1), P-NT, aPD1 loaded diC₁₂-PLGLAG-iRGD (aPD1-L), or P-NT-aPD1 solutions. In the free (CPT+aPD1) group, treatment was administered three times (on days 10, 17 and 24). Flow cytometric analysis was performed on lymphocytes extracted from the tumor on day 25. In vivo bioluminescence imaging of the tumors was observed on days 35 and 60. (4B) The in vivo bioluminescence images of the GL-261 tumors on day 35 and (4C) day 60. (4D) Average tumor growth kinetics of different treatment groups; growth curves were plotted until the first mouse death. Data are given as mean±SD. (n=10 for P-NT-aPD1 treated group, n=5 for other groups). (4E) Survival curves corresponding to different treatment groups. Statistical significance was calculated via the log-rank (Mantel-Cox) test. (4F) Quantification of CD3⁺ T cells and (4G) CD8⁺ T cells infiltration within the tumor between different treatment groups. (4H) Ratios of the tumor infiltrating CD8⁺ T cells (T_{eff}) to T_{reg} cells in the tumors of different treatment groups. Statistical significance was calculated using a two-sided unpaired t-test. Data are given as mean±SD (n=3). *P≤0.05, **P≤0.01, ***P≤0.001.

FIG. 5 illustrates that intra-tumoral delivery of P-NT-aPD1 prodrug hydrogel induces T cell memory against tumor. (5A) The experimental scheme shows the initial treatment schedule as well as the timing of the rechallenge. Mice that considered long-term survival from all treatment groups were rechallenged on the opposite flank in an attempt to develop new tumors. The in vivo bioluminescence imaging of the GL-261 tumors was observed on day 110 (5B) and (5C) on day 130. (5D) Survival curves for naive and rechallenged mice from different treatment groups. Statistical significance was calculated via the log-rank (Mantel-Cox) test. (5E) The percentage of CD8⁺ Tₑₘ cells and (F) CD8⁺ Tₑₘ cells in splenocytes of the naive and rechallenged mice. Statistical significance was calculated using a two-sided unpaired t-test. Data are given as mean±SD (n=3). **P≤0.01, ***P≤0.001.

FIG. 6 shows that P-NT-aPD1 treatment induces systemic antitumor immune response. (6A) Experimental scheme: mice were implanted with GL-261 cells in the right back and left cortical surface, then primary tumors were locally treated with P-NT-aPD1 on day 6. In vivo bioluminescence imaging of the tumors was observed at scheduled time points. (6B) Tumors on the right flank were locally treated with P-NT-aPD1 hydrogel, while intracranial gliomas were designated as ‘distal tumors’ and were left untreated. (n=10 for P-NT-aPD1 treated group, n=5 for Saline group). (6C) In vivo bioluminescence imaging of the GL-261 tumors in response to local P-NT-aPD1 hydrogel treatment. (6D) Survival curves corresponding to Saline and P-NT-aPD1 treated mice. Statistical significance was calculated via the log-rank (Mantel-Cox) test. (6E) Quantification of CD8⁺ T cells infiltration within the tumors of the two treatment groups. (6F) Ratios of the tumor infiltrating T_{eff} to T_{reg} in the tumors of the treatment groups. Statistical significance was calculated using a two-sided unpaired t-test. Data are given as mean±SD (n=3). *P≤0.05, **P≤0.01, ***P≤0.001.

FIG. 7 depicts the characterization of the designed diCPT-iRGD as an in situ-formed anticancer drug-based hydrogel. (7A) Chemical structure of the diCPT-iRGD amphiphile. (7B) RP-HPLC trace and (7C) ESI MS profile of conjugate diCPT-iRGD showing high purity and the expected molecular weight. (7D) Representative TEM images of diCPT-iRGD nanotubes. Scale bar 100 nm. (7E) Pictures of the solution to hydrogel transition of diCPT-iRGD nanotubes after adding PBS.

FIG. 8 depicts the characterization of the designed diCPT-PLGLAG-iRGD as an in situ-formed anticancer drug-based hydrogel. (8A) Chemical structure of the diCPT-PLGLAG-iRGD amphiphile. (8B) RP-HPLC trace and (8C) ESI MS profile of conjugate diCPT-PLGLAG-iRGD showing high purity and the expected molecular weight. (8D) Representative TEM images of diCPT-PLGLAG-iRGD nanotubes (P-NT). Scale bar 100 nm. (8E) Pictures of the solution to hydrogel transition of P-NT after adding PBS.

FIG. 9 depicts the characterization of the designed PTX-iRGD as an in situ-formed anticancer drug-based hydrogel. (9A) Chemical structure of the PTX-iRGD amphiphile. (9B) RP-HPLC trace and (9C) ESI MS profile of conjugate PTX-iRGD showing high purity and the expected molecular weight. (9D) Representative TEM images of PTX-iRGD nanofilaments. Scale bar 100 nm. (9E) Pictures of the solution to hydrogel transition of PTX-iRGD nanofilaments after adding PBS.

FIG. 10 shows the characterization of the anti-PD1 antibody (aPD1) loaded P-NT hydrogel. (10A) Pictures of the formation of aPD1 loaded hydrogel. (10B) Representative confocal 2D and (10C) 2.5D images of a cryosection of aPD1-NT hydrogel. aPD1 was labeled with Cy 3. Scale bar 200 μm.

FIG. 11 shows the characterization of the anti-CD47 (aCD47) antibody loaded PTX hydrogel. (11A) Pictures of the formation of aCD47 loaded hydrogel. (11B) Representative confocal images of a cryosection of aCD47/PTX hydrogel. aCD47 was labeled with Cy 3. Scale bar 200 μm.

FIG. 12 depicts the characterization of the c-di-AMP (CDA) loaded CPT NT hydrogel. (12A) Pictures of the formation of CDA loaded hydrogel. (12B) Representative confocal 2.5D images of a cryosection of CDA-NT hydrogel. CDA was labeled with FITC. Scale bar 200 μm.

FIG. 13 depicts how the biodegradable diCPT-iRGD nanotube hydrogel promotes local retention and extends release of aPD1 in situ. (13A) In vivo gel formation and retention was tested after subcutaneous injection of NT solution in the back of C57BL/6 mice. (13B) A graph reflecting the degradation of the NT hydrogel, the extent of degradation was determined by the mass loss method. (13C) Fluorescence IVIS imaging showing the in vivo retention of aPD1-Cy5.5, administered locally either in solution or encapsulated within NT hydrogel. Experiments were repeated three times. (13D) Fluorescence imaging of tumor tissues and (13E) tumor sections of GL-261 brain tumor-bearing mice that locally received free (CPT+aPD1) or aPD1-NT. Red: Cy5.5-labeled aPD1, Blue: DAPI stained nuclei. Scale bar 200 μm. (13F) Quantification of the in vivo retention profile of Cy5.5-aPD1 and (13G) CPT. Statistical significance was calculated using a two-sided unpaired t-test. Data are given as mean±SD (n=3). *P≤0.05, **P≤0.01, ***P≤0.001.

FIG. 14 illustrates how diCPT-iRGD nanotube hydrogel extends local retention and release of CDA. (14A) Fluorescence IVIS imaging depicting the in vivo retention of CDA-Cy7, administered locally either in solution or after loading into NT hydrogels. Experiments were repeated three times. (14B) Quantification of in vivo retention profile of CDA-Cy7. Statistical significance was determined using a two-sided unpaired t-test. Data are given as mean±SD (n=3). ***P≤0.001. (14C) Fluorescence imaging of tumor sections, showing scanned tumor areas of GL-261 brain tumor-bearing mice that locally received free CDA and CDA-NT. Blue: FITC labeled CDA, Blue: DAPI stained nuclei. Scale bar 200 μm.

FIG. 15 shows that local delivery of aPD1 via CPT-based hydrogels elicits regression of established tumors. (15A) Experimental schedule: GL-261 brain cancer cells were implanted into the right flanks of mice on day 0. Ten days later, mice were intratumorally (it.) injected with free (CPT+aPD1), CPT NT (NT), aPD1 loaded diC₈-iRGD (aPD1(L)) or aPD1-NT. In the free (CPT+aPD1) group, treatment was given three times (on day 10, 17 and 24). Flow cytometric analysis was performed on lymphocytes extracted from the tumor on day 25. In vivo bioluminescence imaging of the tumors as observed on days 35 and 60. (15B) In vivo bioluminescence images of GL-261 tumors as observed on day 35 and (15C) day 60. (15D) Average tumor growth kinetics of different treatment groups, growth curves were plotted until the first mouse death. Data are given as mean±SD. (n=10 for aPD1-NT treated group, n=5 for other groups). (15E) Survival curves corresponding to different treatment groups. Statistical significance was calculated via the log-rank (Mantel-Cox) test. (15F) Absolute number of CD8+ T cells per mg tumor after different treatments. (15G) Ratios of the tumor infiltrating CD8+ T cells (Teff) to CD4+FoxP3+ regulatory T cells (Tregs) cells in the tumors of different treatment groups. Statistical significance was calculated using a two-sided unpaired t-test. Data are given as mean±SD (n=3). *P≤0.05, **P≤0.01.

FIG. 16 shows that local treatment with aPD1-NT hydrogels promoted regression of low-immunogenic 4T1 breast cancers. (16A) Experimental schedule: 4T1 breast cancer cells were implanted into the mammary gland of female BALB/c mice on day 0. Seven days later, mice were intratumorally (it.) injected with free (CPT+aPD1), CPT NT (NT), aPD1-loaded diC₈-iRGD (aPD1-L) or aPD1-NT. In the free (CPT+aPD1) group, treatment was given three times (on day 10, 17 and 24). Flow cytometric analysis was performed for lymphocytes extracted from the tumors on day 25. In vivo bioluminescence imaging of the tumors was observed on days 7 and 28. (16B) In vivo bioluminescence images of the 4T1 tumors in response to different treatments. (16C) Average tumor growth kinetics of different treatment groups. Growth curves were plotted until the first mouse died. Data are given as mean±SD. (n=10 for aPD1-NT treated group, n=5 for other groups). (16D) Survival curves corresponding to different treatment groups. Statistical significance was calculated via the log-rank (Mantel-Cox) test. Statistical significance was calculated using a two-sided unpaired t-test. Data are given as mean±SD (n=3). *P≤0.05, **P≤0.01.

FIG. 17 depicts the effect of local delivery of P-NT-aPD1 hydrogels elicits regression of established CT 26 colon tumors. (17A) Experimental schedule: 5×10⁵ CT 26 colon cancer cells were implanted into the right flanks of BALB/c mice on day 0. Eight days later, mice were intratumorally (it.) injected with free (CPT+aPD1), P-NT, aPD1 loaded diC₁₂-PLGLAG-iRGD (aPD1-L), or P-NT-aPD1 (50 μg aPD1 per mouse; 150 μg CPT per mouse). Mice with long term survival from all treatment groups were re-challenged on the opposite flank to develop new tumors on day 70. (17B) Average tumor growth kinetics of the different treatment groups. (n=6 for aPD1(L) and n=8 for P-NT-aPD1 treated group, n=5 for the other groups). Growth curves were plotted until the first mouse died. Data are given as mean±SD. **P≤0.01. (17C) Survival curves corresponding to the different treatment groups. Statistical significance was calculated via the log-rank (Mantel-Cox) test. **P≤0.01.

FIG. 18 depicts that local delivery of CDA by CPT-based hydrogels can elicit regression of established GL-261 brain tumors. (18A) Experimental schedule: GL-261 brain cancer cells were implanted into the right flanks of the C57BL/6 mice on day 0. Ten days later, the mice were intratumorally (it.) injected with free CPT+CDA, CPT NT (NT), CDA loaded diC8-iRGD NT (CDA-L) and CDA-NT (20 μg CDA per mouse; 100 μg CPT per mouse). Flow cytometric analysis was performed for lymphocytes extracted from tumor on day 13 and day 20. In vivo bioluminescence images of the tumors were taken at scheduled time points. (18B) Survival curves are shown to compare CDA loaded within a hydrogel to free CDA. Statistical significance was determined via the log-rank (Mantel-Cox) test. (18C) The in vivo bioluminescence images of GL-261 tumors as conducted on day 25 and (18D) day 50. (18E) Average tumor growth kinetics in different groups. Growth curves were plotted until the death of the first mouse. Data are given as mean±SD. (n=6-10 for each group). (18F) Survival curves corresponding to different treatment groups. Statistical significance was calculated via the log-rank (Mantel-Cox) test.

FIG. 19 shows local delivery of CDA-NT induces T cell memory and durable antitumor immune response. (19A) The experimental scheme shows the initial treatment schedule as well as the timing of the rechallenge. Mice with long time survival from all groups were rechallenged on the opposite flank to develop new tumors on day 70. (19B) The in vivo bioluminescence imaging of the GL-261 tumors was taken on day 80 and (19C) day 95. (19D) Survival curves for naive and rechallenged mice of different treatment groups. Statistical significance was determined via the log-rank (Mantel-Cox) test. (19E) The percentage of CD8+ Tcm cells and (19F) CD8⁺ Tem cells in splenocytes of the living rechallenged mice.

FIG. 20 shows local treatment with CDA-NT hydrogels promoted regression of low-immunogenic 4T1 breast cancers. (20A) Average tumor growth kinetics in different treatments against 4T1 breast cancer. Growth curves were plotted until the death of the first mouse. Data are given as mean±SD (n=6 for saline treated group, n=8 for other groups). (20B) Survival curves corresponding to different treatment groups. Statistical significance was determined via the log-rank (Mantel-Cox) test. Quantification with flow cytometry gating of (20C) KLRG1⁺ NK cells and (20D) CD103⁺ DCs in different groups on day 13. (20E) Quantification of CD8⁺ T cells in a tumor on day 20. (20F) H&E staining of lung tissues collected from 4T1 tumor-bearing mice after a schedule of different treatments. Scale bar 500 μm (20G) Number of lung metastasis foci after different treatments. (20H) Quantification of CD3⁺ T and (20I) CD8⁺ T cells in the peripheral blood. Statistical significance was calculated using a two-sided unpaired t-test. Data are given as mean±SD (n=3). *P≤0.05, **P≤0.01, ***P≤0.001.

FIG. 21 shows that local delivery of aCD47 by PTX-based hydrogels can elicit regression of established orthotopic GL-261 brain tumors. (21A) In vivo bioluminescence images of the tumors were taken at day 6, (21B) day 13 and (21C) day 20. (21D) Average tumor growth kinetics in different groups. Growth curves were plotted until the death of the first mouse. Data are given as mean±SD. (n=6-10 for each group).

FIG. 22 depicts P-NT-aPD1 treatment inducing systemic antitumor immune response. (22A) Experimental scheme: mice were implanted with GL-261 cells in the right back and left cortical surface, then primary tumors were locally treated with P-NT-aPD1 on day 6. In vivo bioluminescence imaging of the tumors was observed at scheduled time points. (22B) Tumors on the right flank were locally treated with P-NT-aPD1 hydrogel, while intracranial gliomas were designated as ‘distal tumors’ and were left untreated. (n=10 for P-NT-aPD1 treated group, n=5 for Saline group). (22C) In vivo bioluminescence imaging of the GL-261 tumors in response to local P-NT-aPD1 hydrogel treatment. (22D) Survival curves corresponding to Saline and P-NT-aPD1 treated mice. Statistical significance was calculated via the log-rank (Mantel-Cox) test. (22E) Quantification of CD8⁺ T cells gating on CD45⁺ cells infiltration within the tumors of the two treatment groups. (22F) Ratios of the tumor infiltrating T_{eff} to T_{reg} in the tumors of the treatment groups. Statistical significance was calculated using a two-sided unpaired t-test. Data are given as mean±SD (n=3). *P≤0.05, **P≤0.01, ***P≤0.001.

FIG. 23A schematic illustration and characterization of in situ formed alternative chemo-immunotherapeutic supramolecular hydrogel. (23A) Schematics of localized CPT and CDA delivery using a bioresponsive CPT-based nanotube hydrogel for tumor microenvironment regulation and chemo-immunotherapy. (23B) Representative TEM images of diCPT-iRGD nanotubes. Scale bar 50 nm. (23C) Zeta potential measurement of diCPT-iRGD NT solution and CDA condensed NT (CDA-NT) solution. (23D) Photographs of solution-to-hydrogel transition of CDA-loaded nanotubes triggered by addition of PBS. (23E) Representative confocal image of a cryosection of CDA-NT hydrogel. CDA was labeled with FITC. Scale bar 200 μm. (23F) Cumulative release profile of diCPT-iRGD and (23G) CDA from the CDA-NT hydrogels incubated with PBS or 10% FBS at 37° C. Data are given as mean±SD (n=3).

FIG. 24 Biodegradable diCPT-iRGD nanotube hydrogel enables local retention and extended release of CDA. (24A) In vivo gel formation and retention assay was tested after subcutaneous injection of NT solution in the back of C57BL/6 mice. The status of the remaining hydrogel in each mouse was photographed at the indicated time points. (24B) The degradation rate of NT hydrogel, where the extent of degradation was determined by remaining weight (%). (24C) Fluorescence IVIS imaging depicting the in vivo retention of CDA-Cy7, intratumorally injected either in solution form or after loading into NT solutions. Experiments were repeated three times. (24D) Quantification of intratumoral retention profile of CDA-Cy7. Statistical significance was determined using a two-sided unpaired t-test. Data are given as mean±SD (n=3). ***P≤0.001. (24E) Fluorescence imaging of tumor sections at day 3 post free CDA and CDA-NT treatment. Green: FITC labeled CDA, Blue: DAPI stained nuclei. Scale bar: 200 μm.

FIG. 25 depicts local delivery of CDA by CPT-based NT hydrogels eliciting regression of established GL-261 brain tumors. (25A) Experimental schedule: GL-261 brain cancer cells were implanted into the right flanks of the C57BL/6 mice on day 0. Ten days later, the mice were intratumorally (it.) injected with free CPT+CDA, NT, CDA loaded diC₈-iRGD (CDA-L) and CDA-NT (20 μg CDA per mouse; 100 μg CPT equivalent per mouse). Flow cytometric analysis was performed for lymphocytes extracted from tumor at day 3 and day 10 post-treatment. In vivo bioluminescence images of the tumors were taken at scheduled time points. (25B) Survival curves are shown to compare CDA loaded within NT hydrogel to free CDA. Statistical significance was determined via the log-rank (Mantel-Cox) test. (25C) The in vivo bioluminescence images of GL-261 tumors as conducted on day 25 and (25D) day 50 post-tumor inoculation. (25E) Average tumor growth kinetics of GL-261 tumor-bearing mice in different groups. Growth curves were plotted until the death of the first mouse. Data are given as mean±SD. (n=6-10 for each group). (25F) Survival curves corresponding to different treatment groups. Statistical significance was calculated via the log-rank (Mantel-Cox) test. Statistical significance was determined using a two-sided unpaired t-test. *P≤0.05, **P≤0.01, ***P≤0.001.

FIG. 26 shows the important role of STING pathway in stimulating innate and adaptive immune responses for effective tumor regression by CDA-NT. (26A) Quantification with flow cytometry gating of CD69⁺ NK cells, (26B) KLRG1⁺ NK cells, (26C) CD103⁺ DCs and (26D) MHCII⁺ DCs within the C57BL/6 mice-bearing GL-261 tumors at day 3 post-treatment. (26E) Quantification of CD8⁺ T cells within the C57BL/6 mice-bearing GL-261 tumors at day 10 post-treatment. (26F) Ratios of tumor-infiltrating CD8⁺ T cells (T_{eff}) to CD4⁺Foxp3⁺ regulatory T cells (T_{reg}) within the C57BL/6 mice-bearing GL-261 tumors at day 10 post-treatment. (26G) Average tumor growth kinetics of GL-261 tumor-bearing STINGg^{gt/gt} mice treated with Saline, NT, CDA(L) and CDA-NT. Growth curves were plotted until the death of the first mouse. (n=6 for each group). (26H) Average tumor growth kinetics of GL-261 tumor-bearing C57BL/6 mice locally treated with CDA-NT along with NK cells, CD4⁺ T cells and CD8⁺ T cells depletion. Growth curves were plotted until the death of the first mouse (n=8 for each group). Statistical significance was determined using a two-sided unpaired t-test. *P≤0.05, **P≤0.01, ***P≤0.001.

FIG. 27 depicts CDA-NT inducing T cell memory and durable antitumor immune response. (27A) The experimental scheme shows the initial treatment schedule as well as the timing of the rechallenge experiments. Mice with long time survival from all groups were rechallenged on day 70. (27B) The in vivo bioluminescence imaging of the GL-261 tumors was taken on day 80 and (27C) day 95 post-tumor inoculation. (27D) Survival curves for rechallenged mice of different treatment groups. Survival curves for naive mice was given in FIG. 47. Statistical significance was determined via the log-rank (Mantel-Cox) test. (27E) The percentage of CD8⁺ Tₑₘ cells and (27F) CD8⁺ Tₑₘ cells in splenocytes of the living rechallenged mice. Statistical significance was determined using a two-sided unpaired t-test. Data are given as mean±SD (n=3). *P≤0.05, **P≤0.01, ***P≤0.001.

FIG. 28 shows that local treatment with CDA-NT hydrogels promoted regression of low-immunogenic 4T1 breast cancers. 4T1 breast cancer cells were implanted into the mammary gland of female BALB/c mice on day 0. Six days later, mice were intratumorally (it.) injected with free CPT+CDA, NT, CDA loaded diC₈-iRGD (CDA-L) and CDA-NT. (28A) Average tumor growth kinetics in different treatments against 4T1 breast cancer. Growth curves were plotted until the death of the first mouse. Data are given as mean±SD (n=6 for saline treated group, n=8 for other groups). (28B) Survival curves corresponding to different treatment groups. Statistical significance was determined via the log-rank (Mantel-Cox) test. (28C) Quantification with flow cytometry gating of KLRG1⁺ NK cells and (28D) CD103⁺ DCs within the BALB/c mice-bearing 4T1 tumors at day 3 post-treatment. (28E) Quantification of CD8⁺ T cells within the BALB/c mice-bearing 4T1 tumors at day 10 post-treatment. (28F) H&E staining of lung tissues collected from 4T1 tumor-bearing mice after a schedule of different treatments. Scale bar 500 μm (28G) Number of lung metastasis foci in response to different treatments. (28H) Quantification of CD8⁺ T cells in the peripheral blood at day 10 post-treatment. (28I) Quantification of activated IFNγ-secreting CD8⁺ T cells from spleens in response to AH1 peptide derived from gp70. Statistical significance was calculated using a two-sided unpaired t-test. Data are given as mean±SD (n=3). *P≤0.05, **P≤0.01, ***P≤0.001.

FIG. 29 depicts the characterization of the of the diCPT-iRGD amphiphile. (29A) Chemical structure of the diCPT-iRGD amphiphile. (29B) RP-HPLC trace and (29C) ESI MS profile of conjugate diCPT-iRGD showing high purity and the expected molecular weight.

FIG. 30 is a representative TEM image of diCPT-iRGD nanotubes. Scale bar: 100 nm.

FIG. 31 shows images of the solution-to-hydrogel transition of diCPT-iRGD nanotubes alone after adding PBS (no CDA included).

FIG. 32 are graphs depicting in vitro drug release and cytotoxicity of diCPT-iRGD. (32A) Mechanism of GSH induced release of CPT from diCPT-iRGD. (32B) HPLC profile of CPT (blank line), diCPT-iRGD (red line) and diCPT-iRGD incubated with 10 mM GSH (green line) after 2 hours at 37° C. (32C) ESI spectra of peak 1 showing the exacted molecular mass with CPT. (32D) Degradation profiles of diCPT-iRGD in the presence or absence of 10 mM GSH. (32E) In vitro cytotoxicity of free CPT and diCPT-iRGD against GL-261 brain cancer cells. Data are given as mean±SD (n=3).

FIG. 33 depicts inhibition of tumor spheroid growth was evaluated following the treatment with free CPT or NTs. (33A) Representative images of spheroids on different days after treatment. (33B) Volume of tumor spheroids treated with different formulations compared with day 0. Spheroids treated with drug-free DMEM as blank control. Experiments were repeated three times. Data are given as mean±SD (n=7). *P≤0.05, ***P≤0.001.

FIG. 34 shows zeta potential distribution of NTs. (34A) Zeta potential distribution of diCPT-iRGD NT solution before and (34B) after adding CDA.

FIG. 35 is fluorescence IVIS imaging depicting the in vivo retention of CDA-Cy7. CDA-Cy7 intratumoral injected either in solution or after loading into the NT hydrogel and detected by using IVIS on indicated days. Experiments were repeated three times. Please note results collected after day 25 are from three different mice under the same experimental protocols.

FIG. 36 is fluorescence imaging of tumor tissues from GL-261 tumor-bearing mice after 3 days intratumoral injection of free CDA or CDA-NT. CDA was labeled with Cy 7.

FIG. 37 is a graph depicting the quantification of the in vivo retention profile of CPT. Free (CPT+CDA) or CDA-NT was intratumoral injected and the remaining CPT in tumor tissues was detected by HPLC on indicated days. Statistical significance was determined using a two-sided unpaired t-test. Data are given as mean±SD (n=3). ***P≤0.001.

FIG. 38 is the characterization of the designed diCPT-iRGRD amphiphile. Here, iRGRD peptide was used as control for the iRGD peptide, which has the same amino acid composition but differs in sequences. (38A) Chemical structure of the diCPT-iRGRD amphiphile. (38B) RP-HPLC trace and (38C) ESI MS profile of conjugate diCPT-iRGRD showing high purity and the expected molecular weight. (38D) Representative TEM images of diCPT-iRGRD nanotubes. Scale bar: 200 nm. (38E) Pictures of the solution-to-hydrogel transition of diCPT-iRGRD nanotubes after adding PBS.

FIG. 39 is fluorescence imaging of tumor sections to specifically analyze the information from tumor areas of GL-261 tumor-bearing mice that intratumoral injected with NT-Sham (NTs formed from diCPT-iRGRD) or NT. The images were obtained on day 3 day after treatment. Blue: CPT (Excitation at 365 nm), Red: DRAQS™ stained nuclei, scale bar: 500 μm.

FIG. 40 is the characterization of the designed diC₈-iRGD as an in situ-formed, drug-free hydrogel. (40A) Chemical structure of the diC₈-iRGD amphiphile. (40B) RP-HPLC trace and (40C) ESI MS profile of conjugate diC₈-iRGD showing high purity and the expected molecular weight. (40D) Representative TEM images of diC₈-iRGD nanofilaments. Scale bar: 200 nm. (40E) Pictures of the solution-to-hydrogel transition of diC₈-iRGD nanofilaments after adding PBS.

FIG. 41 depicts safety studies of hydrogel treated mice. (41A) Body weight changes of mice in different groups during treatment. Curves were plotted until when the first mouse of the corresponding group died. Data are given as mean±SD. No statistical difference was detected between groups. (41B) Fluorescence IVIS imaging of blood samples from CDA-NT hydrogel treated mice on different days. CDA was labeled with Cy 7. No CDA was detected in the blood. (41C) H&E staining of major organs collected from healthy mice and CDA-NT treated mice at day 30 (scale bar: 200 μm).

FIG. 42 is data showing localized CDA-NT hydrogel had no significant effect on complete blood cell count and serum biochemistry. C57BL/6 mice were sacrificed on day 7 and 15 after CDA-NT treatment. Untreated healthy mice were used as a control. Complete blood cell counts data including: RBC, WBC, PLT, HGB, HCT, MCV, MCH, MCHC, NEUT, LYMPH, MONO and EO. Serum biochemistry data including: ALP, ALT, AST, BUN, CRE and TBIL. Data are presented as mean±SD (n=3). Reference ranges of hematology data of healthy C57BL/6 mice were obtained from Charles River Laboratories.

FIG. 43 shows the expression of type I IFNs and chemokine within the tumor tissues after different treatments. (A) Relative IFNα, (B) IFNβ and (C) CXCL10 chemokine expression in tumor tissue after 3 days of CPT+CDA, NT, CDA(L) and CDA-NT treatment. Statistical significance was determined using a two-sided unpaired t-test. Data are given as mean±SD (n=3). *P≤0.05, **P≤0.01, ***P≤0.001.

FIG. 44 depicts the local delivery of CDA-NT induces both innate and adaptive antitumor immune responses. (44A) Quantification of CD4⁺ T cells and (44B) CD8⁺ T cells infiltration within the tumors at day 3 post-treatment. (44C) Quantification with flow cytometry gating of CD69⁺ NK cells, (44D) KLRG1⁺ NK cells, (44E) CD103⁺ DCs and (44F) MHCII⁺ DCs within the tumors at day 10 post-treatment. (44G) Quantification of CD4⁺ T cells and (44H) IFNγ⁺CD8⁺ T cells infiltration within the tumors at day 10 post-treatment. (44I) Representative flow cytometric images of CD8⁺ T cells infiltration of tumors at day 10 post-treatment. Data are given as mean±SD (n=3). *P≤0.05, **P≤0.01, ***P≤0.001.

FIG. 45 illustrates that CDA-NT shows modest anti-tumor effect on STING-deficient mice. (45A) Quantification of NK cells and (45B) CD8⁺ T cells in GL-261 tumor-bearing STING^{gt/gt} mice at day 10 post-treatment. There is no statistical significance between Saline control and treatment groups. Data are given as mean±SD (n=3). (45C) Survival curves of GL-261 tumor-bearing STING^{gt/gt} mice treated with Saline, NT, CDA(L) and CDA-NT. (n=6 for each group).

FIG. 46 shows that NK cells, CD4⁺ T cells and CD8⁺ T cells are critical to CDA-NT induced tumor growth inhibition. (46A) Depletions of NK cells, CD4⁺ T cells and CD8⁺ T cells were confirmed by flow cytometric analysis of PBMCs after CDA-NT and appropriate antibody treatment. (46B) Survival curves of GL-261 tumor-bearing C57BL/6 mice treated with intratumoral CDA-NT along with NK cells, CD4⁺ T cells and CD8⁺ T cells depletion. (n=8 for each group). Statistical significance was calculated via the log-rank (Mantel-Cox) test.

FIG. 47 is a survival curve for naive and CDA-NT rechallenged mice. Statistical significance was determined via the log-rank (Mantel-Cox) test. ***P≤0.001.

FIG. 48 shows CDA-NT inducing T cell memory. (A) The percentage of CD8⁺ Tₑₘ cells and (B) CD8⁺ Tₑₘ cells in the peripheral blood of the living rechallenged mice. Statistical significance was determined using a two-sided unpaired t-test. Data are given as mean±SD (n=3). *P≤0.05, **P≤0.01, ***P≤0.001.

FIG. 49 shows local delivery of CDA-NT hydrogels can elicit regression of established CT 26 colon tumors. (A) Experimental schedule: CT 26 colon cancer cells were implanted into the right flanks of BALB/c mice on day 0. Eight days later, mice were intratumorally (it.) injected with free CPT+CDA, NT, CDA loaded diC₈-iRGD (CDA-L) and CDA-NT (20 μg CDA per mouse; 100 μg CPT per mouse). Mice with long-term survival from all groups were rechallenged on the opposite flank to develop new tumors on day 70. (B) Average tumor growth kinetics in different groups. Growth curves were plotted until the death of the first mouse. Data are given as mean±SD. (n=8 for CDA (L) and CDA-NT treated group, n=6 for other groups). (C) Survival curves corresponding to different treatment groups. Statistical significance was determined via the log-rank (Mantel-Cox) test. Data are given as mean±SD. *P≤0.05, **P≤0.01.

FIG. 50 shows local treatment with CDA-NT hydrogels promoted regression of low-immunogenic 4T1 breast cancers. (50A) Experimental schedule: 4T1 breast cancer cells were implanted into the mammary gland of female BALB/c mice on day 0. When the tumor volume reached ˜100-150 mm³ at day 6 post-tumor inoculation, mice were intratumorally (it.) injected with free CPT+CDA, NT, CDA loaded diC₈-iRGD (CDA-L) and CDA-NT (20 μg CDA per mouse; 100 μg CPT per mouse). (n=6 for saline treated group, n=8 for other groups). In vivo bioluminescence imaging of the tumors observed at scheduled time points. (50B) The representative in vivo bioluminescence imaging of the 4T1 breast tumors on day 6. (50C) The in vivo bioluminescence imaging of the 4T1 tumors in response to different treatments on day 14 and day 28. It should be noted that although no lung metastasis was observed for the treatment groups by CPT+CDA, NT, and CDA (L) due to the detection limit of IVIS, it actually took place at a reduced level, as evidenced by H&E staining (FIG. 6F) and the presence of cancer nodules (FIG. 6G and FIG. 53).

FIG. 51 is H&E staining of tumor tissues from 4T1 tumor-bearing mice in response to different treatments on day 28. Scale bar=200 μm.

FIG. 52 shows flow cytometric analysis of immune cells change after different treatments on 4T1 tumor-bearing mice. (52A) Representative flow cytometric images of CD8⁺ T cell infiltration within the tumors at day 10 post-treatment. (52B) Representative flow cytometric images of CD8⁺ T cells in the peripheral blood at day 10 post-treatment.

FIG. 53 shows representative lung photographs collected from mice after indicated treatments at day 28 post-tumor inoculation.

FIG. 54 depicts that CDA-NT induced CD8⁺ T cells are critical to inhibit 4T1 tumor growth and metastasis. (54A) Depletions of CD8⁺ T cells were confirmed by flow cytometric analysis of PBMCs after CDA-NT and aCD8 antibody treatment. (54B) Average tumor growth kinetics in different treatments against 4T1 breast cancer. (54C) Representative lung photographs collected from mice after indicated treatments at day 28 post-tumor inoculation. (54D) Number of lung metastasis foci after different treatments at day 28 post-tumor inoculation. (54E) H&E staining of lung tissues collected from 4T1 tumor-bearing mice after indicated treatments at day 28 post-tumor inoculation. Scale bar 500 μm. Data are given as mean±SD. *P≤0.05, **P≤0.01, ***P≤0.001.

FIG. 55 is a graph showing body weight changes of 4T1 tumor-bearing mice in different groups. Curves were plotted until when the first mouse of the corresponding group died. Data are given as mean±SD. No statistical difference was detected between groups.

FIG. 56 shows gating strategies for flow cytometric analysis of tumoral and peripheral lymphocytes. Lymphocyte gate was first used to filter out non-lymphocytic cells, excluded duplets and used L/D stain to exclude dead cells. (56A). Analysis of NK cells. Live CD45⁺ cells were stained with NK1.1 antibody. Gating on NK1.1⁺ cells, the percentage of NK cells that are CD69+ and KLRG1⁺ were identified. (56B). Analysis of DCs. Live CD45⁺ cells were stained with CD11c antibody. Gating on CD11c⁺ cells, the percentage of DCs that are CD103⁺ and MHCII⁺ were identified. (56C). Analysis of CD4 and CD8 cells. Live CD45⁺ cells were stained with CD4 and CD8 antibodies. Gating on CD4+ cells, the percentage of CD4 cells that are FoxP3⁺ were identified. Gating on CD8+ cells, the percentage of cells that are IFNγ⁺ were identified (56D). Analysis of T memory cells. After gating on CD8 cells, CD44 and CD62L antibodies were used to identify the population of cells that were CD44 high CD62L high (T central memory cells) and CD44 high CD62L low (T effector memory cells).

FIGS. 57A-57E show the characterization of a self-assembling paclitaxel hydrogelator. FIG. 57A is the chemical structure of the designed PTX-iRGD prodrug amphiphile. FIG. 57B is a schematic showing localized aCD47 delivery using a PTX filament (PF) hydrogel for the post-resection treatment of glioblastoma. FIG. 57C is an image of representative cryo-TEM of PTX-iRGD filaments. Scale bar 200 nm. FIG. 57D includes images of the solution-to-hydrogel transition of PTX-iRGD filaments induced by addition of PBS. FIG. 57E is graph of storage modulus (G′) and loss modulus (G″) of PF solution as a function of time. The rheological properties were assessed as the PF solution transitioned to a hydrogel upon addition of 10×PBS at 3 minutes.

FIGS. 58A-58J illustrate that a supramolecular filament hydrogel scaffold extends local retention and release of aCD47 in situ. FIG. 58A is a graph of cumulative release profiles of PTX-iRGD and aCD47 from aCD47/PF supramolecular hydrogels. aCD47 was labelled with FITC. FIG. 58B is a graph of free PTX release profiles of 250 μM of PTX-iRGD solution incubated with or without 10 mM GSH. FIG. 58C is a schematic illustration of the proposed PTX release mechanism: hydrogel disruption, filament dissociation followed by liberating the parent PTX in intracellular GSH reductive environment. FIG. 58D includes images showing in vivo gelation and degradation of PTX-iRGD supramolecular hydrogels in C57BL/6 mice. FIG. 58E is a graph showing quantification of the degradation profile of the PTX-iRGD supramolecular hydrogel. Data are given as mean±SD (n=3). FIG. 58F includes fluorescent IVIS images showing the retention of aCD47 in the tumor at the indicated time points following intratumoral injection with free aCD47 or aCD47/PF solution, aCD47 was labelled with Cy5.5. FIG. 58G is a graph showing quantification of the tumoral retention profile of aCD47. Data are given as mean±SD (n=3). FIG. 58H includes fluorescence images of tumor sections from GL-261 tumor-bearing mice that were locally treated with free aCD47 or aCD47/PF on day 3. Green: FITC labeled aCD47, Blue: DAPI stained nuclei. Scale bar 200 μm. FIG. 58I includes ex vivo fluorescence images of brains intracranially injected with aCD47/PF which confirm the retention of aCD47 in the brain. aCD47 was labelled with Cy5.5. FIG. 58J is a graph showing quantification of the retention profile of aCD47 in the brain. Data are given as mean±SD (n=3).

FIGS. 59A-59L illustrate that the aCD47/PF hydrogel elicits regression of GL-261 brain tumors. FIG. 59A includes in vivo bioluminescence images of GL-261 tumors in response to indicated treatments (n=8 for each group). Four representative mice per treatment group are shown. FIG. 59B includes T2 weighted MR images of GL-261 brain tumors at 6-, 20-, and 50-days post injection. Lighter zones, indicated by yellow arrows, show tumor location within the brain. FIG. 59C is a graph showing quantification of tumor growth from bioluminescence imaging of the GL-261 tumors in the indicated treatment groups. FIG. 59D is a graph of survival curves corresponding to the indicated treatment groups. Statistical significance was calculated via the log-rank (Mantel-Cox) test. FIGS. 59E-59G are graphs showing quantification of macrophage (CD11B+F4/80+) (FIG. 59E), CD4⁺ T cell (FIG. 59F)), and CD8⁺ T cell (FIG. 59G) infiltration within the tumor following different treatments. FIG. 59H is a graph of ratios of the tumor infiltrating CD8⁺ T cells (T_{eff}) to T_{reg} cells in the indicated treatment groups. FIGS. 59I-59L are graphs showing secretion levels of IFN-γ (FIG. 59I), TNF-α (FIG. 59J), IL-6 (FIG. 59K), and IL-12 (FIG. 59L) in the indicated treatment groups. Statistical significance was calculated using a two-sided unpaired t-test. Data are given as mean±SD. *P≤0.05, **P≤0.01, ***P≤0.001.

FIGS. 60A-60N illustrate that the PF hydrogel triggers a robust antitumor immunity. FIG. 60A includes representative images and corresponding quantification of flow cytometric analyses of CD45⁺ cells in the indicated treatment groups. FIG. 60B includes representative images and corresponding quantification of flow cytometric analyses of macrophages (CD11B⁺F4/80⁺) within brain tumors in the indicated treatment groups. FIG. 60C is a graph showing secretion levels of IL-12 in the indicated treatment groups. FIGS. 60D-60F are graphs showing quantification of the expression of CD80 (FIG. 60D), CD103 (FIG. 60E) and CD86 (FIG. 60F) in the indicated treatment groups. FIG. 60G includes representative images and corresponding quantification of flow cytometric analyses of CD4⁺ T cell infiltration within the tumor in the indicated treatment groups. FIG. 60H includes representative images and corresponding quantification of flow cytometric analyses of CD8⁺ T cell infiltration within the tumor in the indicated treatment groups. FIGS. 60I and 60J are graphs of secretion levels of IFN-γ (FIG. 60I) and TNF-α (FIG. 60J) in the indicated treatment groups. FIG. 60K is a graph showing quantification of Foxp3⁺ cells gating on CD4⁺ T cells. FIG. 60L includes representative images and relative quantification of flow cytometric analyses of MDSC (CD11b⁺Gr-1⁺) gating on CD45⁺ cells. FIG. 60M is a graph of a representative flow cytometric analysis of surface expression of CD47 on GL-261 brain tumor cells. FIG. 60N includes representative images and relative quantification of flow cytometric analysis of CD47 expression in GL-261 brain tumors after different treatments. Statistical significance was calculated using a two-sided unpaired t-test. Data are given as mean±SD (n=3). *P≤0.05, **P≤0.01, ***P≤0.001.

FIGS. 61A-61I illustrate that aCD47/PF hydrogel inhibits brain tumor recurrence after resection. FIG. 61A includes T2 weighted images of GL-261 brain tumors obtained through MRI showing tumor growth at day 8. Lighter zones indicated by yellow arrows show the tumor location within the brain. FIG. 61B includes images showing procedure of surgical tumor resection from the GL-261 tumor bearing mice and hydrogel implantation. FIG. 61C includes images of H&E staining of brain sections collected from the GL-261-bearing mice immediately following surgical tumor removal. RC indicates the resection cavity. Scale bar: left, 500 μm; right, 100 μm. FIG. 61D includes representative images and quantitative analysis showing the extent of surgical resection using bioluminescence imaging. FIG. 61E is a graph of survival curves of GL-261 brain tumor-bearing mice with or without surgical resection (n=8). Arrow indicates the time of the surgery. Statistical significance was determined via the log-rank (Mantel-Cox) test. FIG. 61F includes in vivo bioluminescence images of surgically treated GL-261 tumors in response to indicated treatments (n=8 for each group). Four representative mice per treatment group are shown. FIG. 61G includes T2 weighted MR images of surgically treated GL-261 brain tumors at day 30 and 60 post tumor implantation. FIG. 61H is a graph showing quantification of bioluminescence imaging of the surgically treated GL-261 tumors in the indicated treatment groups. FIG. 61I is a graph showing survival curves of surgically treated GL-261 brain tumor-bearing mice corresponding to indicated treatments. Statistical significance was calculated via the log-rank (Mantel-Cox) test. Data are given as mean±SD. *P≤0.05, **P≤0.01, ***P≤0.001.

FIGS. 62A-62F illustrate that the aCD47/PF hydrogel induces durable antitumor immune response. FIG. 62A is the experimental scheme showing the treatment schedule as well as the timing of the rechallenge. FIG. 62B includes in vivo bioluminescence images of the GL-261 tumors of naive and rechallenged mice taken on days 6, 13, and 20 post tumor rechallenge. FIG. 62C is a graph showing quantification of bioluminescence imaging of naive and rechallenged mice in the indicated treatment groups. FIG. 62D is a graph of survival curves for naive and rechallenged mice corresponding to the indicated treatment groups. Statistical significance was determined via the log-rank (Mantel-Cox) test. FIGS. 62E and 62F are graphs showing the percentage of CD4⁺ Tₑₘ cells (FIG. 62E) and CD8⁺ Tₑₘ cells (FIG. 62F) in splenocytes of the naïve and rechallenged mice. Statistical significance was determined using a two-sided unpaired t-test. Data are given as mean±SD (n=3). *P≤0.05, **P≤0.01, ***P≤0.001.

### DETAILED DESCRIPTION OF THE INVENTION

The present disclosure is predicated, at least in part, on the generation of a prodrug containing a cytotoxic agent (e.g., a chemotherapeutic agent) conjugated to a hydrophilic moiety, such as peptide or polypeptide. When complexed with an immunomodulator (e.g., a checkpoint inhibitor), the prodrug can self-assemble into hydrogels at a tumor site, followed by long-term release of the cytotoxic agent and the immunomodulator. Release of the cytotoxic agent and immunomodulator results in killing of cancer cells and elicits an immune-stimulating tumor microenvironment which sensitizes against cancer recurrence.

The disclosure demonstrates that local administration of a composition described herein can self-assemble into a nanofiber hydrogel, which induces durable T cell memory and robust systemic antitumor immunity, which may be used to prevent tumor recurrence and potential metastasis. Thus, the present inventive compositions and methods of local therapy with immunomodulators can enhance antitumor immune responses and improve the overall response rate.

In some embodiments, the compositions and methods described herein may lower the dosage of immunomodulator required for therapeutic efficacy and minimize systemic exposure. Moreover, the use of the disclosed prodrug hydrogelators to deliver an immunomodulator (e.g., an immune checkpoint inhibitor) eases concerns about any possible short- or long-term toxicities of synthetic or natural drug carriers, representing a simple yet effective means to achieve combination chemo- and immuno-therapy with translation potential. In some embodiments, the incorporation of an MMP-2 responsive substrate and an iRGD segment in the prodrug design contributes to the improved treatment efficacy and reduced side effects.

The present disclosure therefore describes a combined chemo-immunotherapy strategy on the basis of a prodrug hydrogel to boost immunity against cancer. This in situ-formed prodrug hydrogel can serve as a therapeutic reservoir for sustained intratumoral release of both a cytotoxic agent (e.g., chemotherapeutic agent) and an immunomodulator (e.g., an immune checkpoint inhibitor). The prodrug hydrogel increases the frequency of T effector cells and reduces the population of immune suppressor cells, so as to provoke a robust antitumor immune response. The long-term memory T cell and systemic immune response induced by local delivery of the prodrug composition suggests that this platform may be used to treat tumor recurrence and metastasis.

### Prodrug Compositions

In some embodiments, the disclosure provides a composition comprising: (a) a prodrug comprising one or more cytotoxic agents conjugated to a hydrophilic moiety by a linker, and (b) one or more immunomodulators. The term “prodrug,” as used herein, refers to a biologically inactive compound which can be metabolized in a subject (e.g., a human) to produce a pharmacologically active compound (e.g., a drug). Prodrugs may be used to improve how a compound is absorbed, distributed, metabolized, and excreted by the subject.

An agent is “cytotoxic” and induces “cytotoxicity” if the agent kills or inhibits the growth of cells, particularly cancer cells. In some embodiments, for example, cytotoxicity includes preventing cancer cell division and growth, as well as reducing the size of a tumor or cancer. Cytotoxicity of cancer cells may be measured using any suitable cell viability assay known in the art, such as, for example, assays which measure cell lysis, cell membrane leakage, and apoptosis. For example, methods including but not limited to trypan blue assays, propidium iodide assays, lactate dehydrogenase (LDH) assays, tetrazolium reduction assays, resazurin reduction assays, protease marker assays, 5-bromo-2′-deoxy-uridine (BrdU) assays, and ATP detection may be used. Cell viability assay systems that are commercially available also may be used and include, for example, CELLTITER-GLO® 2.0 (Promega, Madison, Wis.), VIVAFIX™ 583/603 Cell Viability Assay (Bio-Rad, Hercules, Calif.); and CYTOTOX-FLUOR™ Cytotoxicity Assay (Promega, Madison, Wis.).

Any cytotoxic agent that kills cancer cells or inhibits cancer cell proliferation may be included in the prodrug. Examples of cytotoxic agents include, but are not limited to, alkylating agents, antibiotics, antimetabolites, free radical generators and mitotic inhibitors. It will be appreciated that cytotoxic agents may also be referred to in the art as “cytotoxic chemotherapeutics” or simply “chemotherapeutics.” Thus, in some embodiments, the cytotoxic agent employed in the disclosed prodrug comprises one or more chemotherapeutics. Examples of cytotoxic chemotherapeutics include, but are not limited to, alkylating agents, nitrogen mustard alkylating agents, nitrosourea alkylating agents, antimetabolites, purine analog antimetabolites, pyrimidine analog antimetabolites, hormonal antineoplastics, natural antineoplastics, antibiotic natural antineoplastics, and vinca alkaloid natural antineoplastics, such as carboplatin and cisplatin. More specifically, chemotherapeutic agents that may be employed in the disclosed prodrug include, for example, goserelin, leuprolide, tamoxifen, aldesleukin, tretinoin (ATRA), adriamycin, asparaginase, bleomycin, busulphan, cisplatin, carboplatin, carmustine, capecitabine, chlorambucil, cytarabine, cyclophosphamide, camptothecin (CPT), dacarbazine, dactinomycin, daunorubicin, dexrazoxane, docetaxel, doxorubicin, etoposide, floxuridine, fludarabine, fluorouracil, gemcitabine, hydroxyurea, idarubicin, ifosfamide, irinotecan, lomustine, mechlorethamine, mercaptopurine, meplhalan, methotrexate, mitomycin, mitotane, mitoxantrone, nitrosurea, paclitaxel, pamidronate, pentostatin, plicamycin, procarbazine, rituximab, streptozocin, teniposide, thioguanine, thiotepa, vinblastine, vincristine, vinorelbine, taxol, transplatinum, anti-vascular endothelial growth factor compounds (“anti-VEGFs”), anti-epidermal growth factor receptor compounds (“anti-EGFRs”), 5-fluorouracil, etc. In some embodiments, the cytotoxic agent is camptothecin, paclitaxel, bumetanide, verteporfrin, or vorapaxar.

The type and number of chemotherapeutics used in the disclosed prodrug composition will depend on the standard chemotherapeutic regimen for a particular tumor type. In some embodiments, two or more different cytotoxic agents may be included in the prodrug. For example, two different chemotherapeutic agents can be included in a single prodrug. In other embodiments, three or four different cytotoxic agents may each be linked by a biodegradable linker to the hydrophilic moiety of the prodrug of the present invention.

In some embodiments, the cytotoxic agent or drug (D) acts as a hydrophobic portion of the prodrug compositions of the present invention. As used herein, the term “hydrophobic” biologically active agents or drug molecules describes a heterogeneous group of molecules that exhibit poor solubility in water but that are typically, but certainly not always, soluble in various organic solvents. Often, the terms slightly soluble (1-10 mg/ml), very slightly soluble (0.1-1 mg/ml), and practically insoluble (<0.1 mg/ml) are used to categorize such substances. Drugs such as steroids and many anticancer drugs (e.g., chemotherapeutic agents) are important classes of poorly water-soluble drugs; however, their water solubility varies over at least two orders of magnitudes. Typically, such molecules require secondary solubilizers such as carrier molecules, liposomes, polymers, or macrocyclic molecules such as cyclodextrins to help the hydrophobic drug molecules dissolve in aqueous solutions necessary for drug delivery in vivo. Other types of hydrophobic drugs show even a lower aqueous solubility of only a few ng/ml. Since insufficient solubility commonly accompanies undesired pharmacokinetic properties, the high-throughput screening of kinetic and thermodynamic solubility as well as the prediction of solubility is of major importance in discovery (lead identification and optimization) and development.

While the prodrug ideally comprises a cytotoxic agent as described above, in some embodiments the prodrug may comprise other types of biologically active agents. As used herein, the term “biologically active agent” includes any compound or molecule for treating tumor-related diseases, e.g. drugs, inhibitors, and proteins. An active agent and a biologically active agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc.

As used herein, the term “biologically active agent” can also include imaging agents for use in identifying the location of the molecules in the tissues. In accordance with an embodiment, the imaging agent is a fluorescent dye. The dyes may be emitters in the visible or near-infrared (NIR) spectrum. Known dyes useful in the present invention include carbocyanine, indocarbocyanine, oxacarbocyanine, thiocarbocyanine and merocyanine, polymethine, coumarine, rhodamine, xanthene, fluorescein, boron-dipyrromethane (BODIPY), Cy5, Cy5.5, Cy7, VivoTag-680, VivoTag-S680, VivoTag-S750, AlexaFluor660, AlexaFluor680, AlexaFluor700, AlexaFluor750, AlexaFluor790, Dy677, Dy676, Dy682, Dy752, Dy780, DyLight547, Dylight647, HiLyte Fluor 647, HiLyte Fluor 680, HiLyte Fluor 750, IRDye 800CW, IRDye 800RS, IRDye 700DX, ADS780WS, ADS830WS, and ADS832WS.

Organic dyes which are active in the NIR region are known in biomedical applications. However, there are only a few NIR dyes that are readily available due to the limitations of conventional dyes, such as poor hydrophilicity and photostability, low quantum yield, insufficient stability and low detection sensitivity in biological system, etc. Significant progress has been made on the recent development of NIR dyes (including cyanine dyes, squaraine, phthalocyanines, porphyrin derivatives and BODIPY (borondipyrromethane) analogues) with much improved chemical and photostability, high fluorescence intensity and long fluorescent life. Examples of NIR dyes include cyanine dyes (also called as polymethine cyanine dyes) are small organic molecules with two aromatic nitrogen-containing heterocycles linked by a polymethine bridge and include Cy5, Cy5.5, Cy7 and their derivatives. Squaraines (often called Squarylium dyes) consist of an oxocyclobutenolate core with aromatic or heterocyclic components at both ends of the molecules, an example is KSQ-4-H. Phthalocyanines, are two-dimensional 18n-electron aromatic porphyrin derivatives, consisting of four bridged pyrrole subunits linked together through nitrogen atoms. BODIPY (borondipyrromethane) dyes have a general structure of 4,4′-difluoro-4-bora-3a, 4a-diaza-s-indacene) and sharp fluorescence with high quantum yield and excellent thermal and photochemical stability.

Other imaging agents which may be employed in the compositions of the present invention include PET and SPECT imaging agents. The most widely used agents include branched chelating agents such as di-ethylene tri-amine penta-acetic acid (DTPA), 1,4,7,10-tetra-azacyclododecane-1,4,7,10-tetraacetic acid (DOTA) and their analogs. Chelating agents, such as di-amine dithiols, activated mercaptoacetyl-glycyl-glycyl-gylcine (MAG3), and hydrazidonicotinamide (HYNIC), are able to chelate metals like ^{99m}Tc and ¹⁸⁶Re. Instead of using chelating agents, a prosthetic group such as N-succinimidyl-4-¹⁸F-fluorobenzoate (¹⁸F-SFB) is necessary for labeling peptides with ¹⁸F. In accordance with a preferred embodiment, the chelating agent is DOTA.

Various forms of the biologically active agents may be used. These include, without limitation, such forms as uncharged molecules, molecular complexes, salts, ethers, esters, amides, prodrug forms and the like, which are biologically activated when implanted, injected or otherwise placed into a subject.

A moiety, substance, compound, or molecule is “hydrophilic” when its interactions with water and other polar substances are more thermodynamically favorable than its interactions with oil or other hydrophobic solvents. A hydrophilic agent is typically charge-polarized and capable of hydrogen bonding, which makes it soluble not only in water but also in other polar solvents. In some cases, both hydrophilic and hydrophobic properties occur in a single molecule, and such molecules are referred to as “amphiphilic” molecules or “amphiphiles.” Examples of amphiphilic molecules include, but are not limited to, lipids that form cell membrane, soaps, alcohols, and certain amino acids.

The hydrophilic moiety may be any molecule or portion of a molecule that confers a self-assembly feature to the disclosed prodrug. Exemplary moieties include, but are not limited to, peptides, polypeptides, and oligo ethyleneoxide (OEG). Any suitable hydrophilic peptide or polypeptide may be included in the prodrug, but ideally the peptide or polypeptide comprises a biological activity such as tumor targeting, tissue penetrating, cell penetrating, apoptosis-inducing, and/or is capable of binding to known cellular epitopes, such as integrins or cancer cell receptors, and derivatives, or functional fragments or functional homolog of such peptides. The terms “peptide,” “polypeptide,” and “protein” are used interchangeably herein and refer to a polymeric form of amino acids of any length, which can include coded and non-coded amino acids, chemically or biochemically modified or derivatized amino acids, and polypeptides having modified peptide backbones. Oligo(ethylene glycol) (OEG) chains are hydrophilic and have been widely used as side chains of conjugated polymers. In addition to hydrophilicity, OEG chains exhibit high polarity, high flexibility, and ionic conductivity.

The term, “amino acid” includes the residues of the natural α-amino acids (e.g., Ala, Arg, Asn, Asp, Cys, Glu, Gln, Gly, His, Lys, Ile, Leu, Met, Phe, Pro, Ser, Thr, Trp, Tyr, and Val) in D or L form, as well as β-amino acids, synthetic and non-natural amino acids. Many types of amino acid residues are useful in the polypeptides and the invention is not limited to natural, genetically-encoded amino acids. Examples of amino acids that can be utilized in the peptides described herein can be found, for example, in Fasman, 1989, CRC Practical Handbook of Biochemistry and Molecular Biology, CRC Press, Inc., and the reference cited therein. Another source of a wide array of amino acid residues is provided by the website of RSP Amino Acids LLC.

Reference herein to “derivatives” includes parts, fragments and portions of the hydrophilic moiety of the prodrug molecule. For peptides and polypeptides, a derivative also includes a single or multiple amino acid substitution, deletion and/or addition. Homologues include functionally, structurally or stereochemically similar peptides from the naturally occurring peptide or protein. All such homologs are contemplated by the present invention.

Analogs and mimetics include molecules which contain non-naturally occurring amino acids or which do not contain amino acids but nevertheless behave functionally the same as the peptide. Examples of non-natural amino acids and amino acid derivatives include, but are not limited to, use of norleucine, 4-amino butyric acid, 4-amino-3-hydroxy-5-phenylpentanoic acid, 6-aminohexanoic acid, t-butylglycine, norvaline, phenylglycine, omithine, sarcosine, 4-amino-3-hydroxy-6-methylheptanoic acid, 2-thienyl alanine and/or D-isomers of amino acids. Natural product screening is one useful strategy for identifying analogs and mimetics.

Exemplary hydrophilic peptides or polypeptides include, but are not limited to, RGD or RGDR (SEQ ID NO: 1), HDK, iRGD (cCRGDKGPDC) (SEQ ID NO: 2), or derivatives thereof, having z=1 to 6 repeating moieties. As used herein, the term “iRGD” refers to 9-amino acid cyclic (c) peptide (sequence: CRGDKGPDC) (SEQ ID NO: 2) molecular mimicry agent that was originally identified in an in vivo screening of phage display libraries in tumor-bearing mice. The peptide is able to home to tumor tissues, but in contrast to standard RGD (arginylglycylaspartic acid) peptides, iRGD is distributed much more extensively into extravascular tumor tissue. It was later identified that this extravasation and transport through extravascular tumor tissue was due to the bi-functional action of the molecule: after the initial RGD-mediated tumor homing, another pharmacological motif is able to manipulate tumor microenvironment, making it temporarily accessible to circulating drugs. This second step is mediated through specific secondary binding to neuropilin-1 receptor, and subsequent activation of a trans-tissue pathway, dubbed the C-end Rule (CendR) pathway. It is believed that the RGD sequence motif mediates binding to αVβ3 and αV5 integrins that are expressed on tumor endothelial cells. Second, upon αV binding, a protease cleavage event is activated, revealing the c-terminal CendR motif (R/KXXR/K) (SEQ ID NO: 3) of the peptide. The CendR motif is then able to bind to neuropilin-1, activating an endocytotic/exocytotic transport pathway. Other hydrophilic peptides which can be used in conjunction with the compositions of the present invention include tumor associated antigens, such as, for example, CEA, TAG-72, CyclinB 1, Ep-CAM, Her2/neu, CDK4, fibronectin, p53, ras, etc.

The one or more cytotoxic agents of the disclosed prodrug may be conjugated to a hydrophilic moiety (e.g., a peptide or polypeptide) by a linker. A linker is any chemical moiety that is capable of linking one compound, usually a drug, to another compound (e.g., a cell-binding agent such as an peptide ligand or antibody) in a stable, covalent manner. Linkers can be susceptible to or be substantially resistant to acid-induced cleavage, light-induced cleavage, peptidase-induced cleavage, esterase-induced cleavage, and disulfide bond cleavage, at conditions under which the antibody remains active. In some embodiments, the linker can be any amino acid with a side chain having a free amino, carboxyl or disulfide group. Exemplary amino acids useful as amino acid linkers in the nanofiber hydrogels of the present invention include lysine (K), glutamic acid (E), arginine (R) and cysteine (C).

Suitable linkers are well known in the art and include, for example, disulfide groups, thioether groups, acid labile groups, photolabile groups, peptidase labile groups, and esterase labile groups. Linkers also include charged linkers, and hydrophilic forms thereof as described herein and known in the art. In some embodiments, the linker may be a cleavable linker, a non-cleavable linker, a hydrophilic linker, and a dicarboxylic acid-based linker. It is contemplated that the cytotoxic agent is covalently linked to a hydrophilic moiety via a biodegradable bond. For example, amino groups, carboxyl groups and disulfide bonds are capable of being cleaved in vitro by various chemical and biological or enzymatic processes. In some embodiments, the linker comprises at least one disulfide group (referred to as a “disulfide linker”). For example, in some embodiments, the linker can comprise a C₁-C₆ acyl-disulfide group. For example, the linker can be (4-(pyridin-2-yldisulfanyl)butanoate) (buSS), or (4-(pyridin-2-yldisulfanyl)ethyl carbonate) (etcSS). Other linkers that may be employed include, but are not limited to, N-succinimidyl 4-(2pyridyldithiojpentanoate (SPP); N-succinimidyl 4-(2-pyridyldithio)-2-sulfopentanoate (sulfoSPP); N-succinimidyl 4-(2-pyridyldithio)butanoate (SPDB); N-succinimidyl 4-(2-pyridyldithio)2-sulfobutanoate (sulfo-SPDB); N-succinimidyl 4-(maleimidomethyl) cyclohexanecarboxylate (SMCC); N-sulfosuccinimidyl 4-(maleimidomethyl) cyclohexanecarboxylate (sulfoSMCC); N-succinimidyl-4-(iodoacetyl)-aminobenzoate (SIAB); and N-succinimidyl-[(Nmaleimidopropionamidoj-tetraethyleneglycol] ester (NHS-PEG4-maleimide).

In other embodiments, the linker further comprises a matrix metalloproteinase-2 (MMP-2) cleavable peptide (also referred to herein as an “MMP-2 degradable peptide”). Matrix metalloproteinases (MMPs) are major extracellular enzymes involved in cancer initiation, progression, and metastasis. MMPs are widely used as cancer biomarkers and therapeutic targets. Recently, MMPs have been investigated as robust tumor microenvironmental stimuli for ‘smart’ MMP-responsive drug delivery and tumor targeting and have shown great potential in cancer diagnosis and therapy (see, e.g., Yao et al., Trends in Pharmacological Sciences, 39(8): 766-781 (2018); doi.org/10.1016/j.tips.2018.06.003). For example, because MMPs cleave active agents from the extracellular matrix by degrading proteins, MMPs have been used to cleave prodrugs and thus release the active drug selectively in the diseased tissue overexpressing MMPs (e.g., cancers). MMP-activated peptide prodrugs have been generated, in which therapeutic drugs are attached to an MMP substrate peptide. When the peptide is cleaved, the drug becomes active. The prodrug can be orally available or parenterally administered and targeting depends only on the peptide sequence specificity. Many such prodrugs have been designed for various anti-cancer agents (see, e.g., U.S. Pat. Nos. 6,844,318, 6,855,689, and 5,659,061; and Timar et al., Cancer Chemother Pharmacol., 41(4): 292-8 (1998)). There has been a growing interest in the development of peptide-based supramolecular filaments responsive to specific MMPs due to their important biomedical applications (Yang et al., Soft Matter 2009, 5, 2546-2548; Giano et al, Biomaterials 2011, 32, 6471-6477; Galler et al., J. Am. Chem. Soc. 2010, 132, 3217-3223; and Chau et al., Biomaterials 2008, 29, 17131719).

Any suitable peptide that can be cleaved or degraded by MMP-2 may be employed in the prodrug described herein. In some embodiments the MMP-2 cleavable peptide comprises one or more of the following amino acid sequences PLGLAG (SEQ ID NO: 4), PLGVR (SEQ ID NO: 5), or GPLGIAGQ (SEQ ID NO: 6), or functional variants or derivatives thereof. Without being limited to any particular example, the composition of the present invention can be a hetero-dual drug amphiphile comprising a first cytotoxic agent and a second cytotoxic agent linked by the same or different linker, for example buSS, to the peptide portion of the prodrug.

The composition further comprises one or more immunomodulators. The terms “immune modulator,” “immune modulator protein,” and “immunomodulator,” may be used interchangeably to refer to a substance or protein that affects normal immune function of an organism. In some embodiments, an immune modulator stimulates immune functions of an organism, such as by activating, boosting, or restoring immune responses. In other embodiments, an immune modulator may exert a negative effect on immune function, such as by attenuating an existing immune response or preventing the stimulation of an immune response. Immunomodulators may be naturally occurring substances (e.g., proteins) or may be synthetically generated compounds. Examples of naturally occurring immunomodulators include, but are not limited to, cytokines, chemokines, and interleukins. Cytokines are small proteins (˜25 kDa) that are released by a variety of cell types, typically in response to an activating stimulus, and induce responses through binding to specific receptors. Examples of cytokines include, but are not limited to, interferons (i.e., IFN-α, IFN-β, IFN-γ), leukemia inhibitory factor (LIF), oncostatin M (OSM), granulocyte-macrophage colony stimulating factor (GM-CSF), granulocyte colony stimulating factor (G-CSF), tumor necrosis factors (e.g., TNF-α), transforming growth factor (TGF)-β family members (e.g., TGF-β1 and TGF-β2). Chemokines are a class of cytokines that have chemoattractant properties, inducing cells with the appropriate receptors to migrate toward the source of the chemokine. Chemokines fall mainly into two groups: CC chemokines comprising two adjacent cysteines near the amino terminus, or CXC chemokines, in which two equivalent cysteine residues are separated by another amino acid. CC chemokines include, but are not limited to, chemokine ligands (CCL) 1 to 28, and CXC chemokines include, but are not limited to, CXC ligands (CXCL) 1 to 17. Interleukins are a structurally diverse group of cytokines which are secreted by macrophages in response to pathogens and include, for example, interleukin-1 (IL-1), IL-2, IL-6, IL-12, and IL-8. Other cytokines, chemokines, and interleukins are known in the art (see, e.g., Cameron M. J., and Kelvin D. J., Cytokines, Chemokines and Their Receptors. In: Madame Curie Bioscience Database, Austin (Tex.): Landes Bioscience; 2000-2013). In other embodiments, an immunomodulator may be synthetically or recombinantly generated. For example, an immunomodulator may be a fusion protein, a chimeric protein, or any modified version of a naturally-occurring immune modulator.

The immunomodulator may be an immune checkpoint regulator. Immune checkpoints are molecules on immune cells that must be activated or inhibited to stimulate immune system activity. Tumors can use such checkpoints to evade attacks by the immune system. The immune checkpoint regulator may be an antagonist of an inhibitory signal of an immune cell, also referred to as a “checkpoint inhibitor,” which blocks inhibitory checkpoints (i.e., molecules that normally inhibit immune responses). For example, the immune checkpoint regulator may be an antagonist of A2AR, BTLA, B7-H3, B7-H4, CTLA4, GALS, IDO, KIR, LAG3, PD-1, TDO, TIGIT, TIM3 and/or VISTA. Checkpoint inhibitor therapy therefore can block inhibitory checkpoints, restoring immune system function. Currently approved checkpoint inhibitors target the molecules CTLA4, PD-1, and PD-L1, and include ipilimumab (YERVOY®), nivolumab (OPDIVO®), pembrolizumab (KEYTRUDA®), atezolizumab (TECENTRIQ®), avelumab (BAVENCIO®), and durvalumab (IMFINZI®). Any suitable checkpoint inhibitor, such as those described in, e.g., Kyi, C. and M. A. Postow, Immunotherapy, 8(7): 821-37 (2016); Collin, M., Expert Opin Ther Pat., 26(5): 555-64 (2016); Pardoll, D. M., Nat Rev Cancer, 12(4): 252-6 (2012); and Gubin et al., Nature, 515(7528): 577-81 (2014)) may be included in the disclosed compositions. In some embodiments, the immune checkpoint inhibitor is a monoclonal antibody, such as an anti-CTLA-4 antibody, an anti-B7-H4 antibody, an anti-B7-H1 antibody, an anti-LAG3 antibody, an anti-CD47 antibody, or an anti-PD1 antibody. More particularly, the immune checkpoint inhibitor is an anti-CD47 antibody (also referred to herein as “aCD47”) or an anti-PD1 antibody (also referred to herein as “aPD1”). In other embodiments, the immune checkpoint regulator may be an agonist of an immune cell stimulatory receptor, such as an agonist of BAFFR, BCMA, CD27, CD28, CD40, CD122, CD137, CD226, CRTAM, GITR, HVEM, ICOS, DR3, LTBR, TACI and/or OX40.

PD-1 ligands (PD-L1 and PD-L2) expressed on the surface of tumor cells and antigen presenting cells (APCs) engage PD-1+ T cells, resulting in T cell apoptosis, anergy, and exhaustion. Blocking these interactions between PD-1 and its ligands with an anti-PD-1 antibody (aPD1) leads to restoration of T cell function and long-term antitumor immune response. Despite its promising potential, current aPD1 therapy benefits a relatively small fraction of patients, exhibiting only a 10 to 30% treatment response rate. This is partially because patients with non-immunogenic (cold) tumors, which are characterized by insufficient infiltration of tumor antigen-specific T cells (primarily CD8⁺ T cells) and low tumoral expression of PD-L1, respond poorly to immune checkpoint inhibitors. Furthermore, systemic administration as a conventional means results in off-target binding of the antibodies to normal tissues, compromising the efficacy of aPD1 therapy and inducing severe immune-related side effects. Consequently, to fully realize the potential of immune checkpoint inhibitors, approaches are needed to convert cold tumors to immune-stimulating tumors and to sensitize tumors to aPD1 with minimal off-target adverse events. Examples of anti-PD1 antibodies include, but are not limited to, pembrolizumab, nivolumab, cemiplimab, avelumab, durvalumab, atezolizumab, spartalizumab (PDR001), camrelizumab (SHR1210), sintilimab (IBI308), tislelizumab (BGB-A317), toripalimab (JS 001), MPDL3280A, MEDI4736, AMP-224, AMP-514, and MSB0010718C.

CD47 is an immunoglobulin that is overexpressed on the surface of many types of cancer cells. CD47 forms a signaling complex with signal-regulatory protein a (SIRPα), enabling the escape of these cancer cells from macrophage-mediated phagocytosis. In recent years, CD47 has been shown to be highly expressed by various types of solid tumors and to be associated with poor patient prognosis in various types of cancer. A growing number of studies have since demonstrated that inhibiting the CD47-SIRPα signaling pathway promotes the adaptive immune response and enhances the phagocytosis of tumor cells by macrophages. Indeed, blocking CD47-SIRPα interaction with an anti-CD47 antibody (aCD47) can promote tumor cell phagocytosis, and at the same time, trigger antitumor T cell immune response. Exemplary anti-CD47 monoclonal antibodies include, but are not limited to, Hu5F9-G4 (Liu et al., PLoS One.; 10(9): e0137345 (2015)), AO-176 (Puro et al., Mol Cancer Ther, 19(3) 835-846 (2020); DOI: 10.1158/1535-7163.MCT-19-1079), and commercially available antibodies (e.g., Cat. No. 127519, BioLegend, Inc., San Diego, Calif.).

In other embodiments, the immunomodulator may be a small molecule. The term “small molecule,” as used herein, refers to a low molecular weight (<900 daltons) organic compound that may regulate a biological process, with a size typically on the order of 1 nm. Small molecules exhibit a variety of biological functions and may serve a variety applications, such as in cell signaling, as pharmaceuticals, and as pesticides. Examples of small molecules include amino acids, fatty acids, phenolic compounds, alkaloids, steroids, bilins, retinoids, etc.

Recent work has demonstrated that activation of the stimulator of interferon genes (STING) signal pathway vigorously stimulates innate and adaptive immune responses in the TME, which has been proposed to enhance tumor immunogenicity. It is also well-known that chemotherapeutic drugs such as camptothecin (CPT) and etoposide can trigger cell death by instigating DNA damage. It has been reported that these anticancer drugs can lead to release of fragmented DNA into the cytosol and stimulation of intrinsic STING-dependent activity, suggesting that these small molecule drugs may exert their anticancer effects partly through triggering the STING pathway and activating the immune system.

Thus, in some embodiments, the small molecule is an agonist of the stimulator of interferon genes (STING) protein. STING is a cytosolic adaptor protein that responds to endogenous and exogenous cytosolic nucleic acid ligands. Cytosolic DNA is recognized by cyclic GMP-AMP synthetase (cGAS), which catalyzes the generation of the secondary messenger cGAMP. cGAMP binds to STING and leads to the production of type I interferons (IFNs) and other cytokines. Type I IFNs selectively stimulate antigen-presenting cells (in particular dendritic cells, DCs), which in turn prime and activate tumor antigen-specific T cells. Moreover, activation of the STING pathway in tumor cells can lead to natural killer (NK) cells infiltrating into the tumor and mobilizing an antitumor response. In light of these discoveries, STING agonists, such as cGAMP and cyclic dinucleotides (CDNs), have been utilized to promote an immune-stimulating TME and augment antitumor immune responses. While promising, CDNs as a monotherapy have shown poor therapeutic benefit in preclinical trials due to its high toxicity, requiring multiple doses and combination with immune checkpoint blockade. Examples of STING agonists that may be used in the disclosed compositions include, but are not limited to, cGAMP and cyclic dinucleotides, 2′3′-cGAMP (cyclic [G(2′,5′)pA(3′,5′)p]), 2′2′-cGAMP, cyclic [G(2′,5′)pA(2′,5′)p], 3′3′-cGAMP (cyclic [G(3′,5′)pA(3′,5′)p] (cGAMP), Bis-(3′-5′)-cyclic dimeric adenosine monophosphate (c-di-AMP), Bis-(3′-5′)-cyclic dimeric guanosine monophosphate (c-di-GMP), cyclic (adenine monophosphate-inosine monophosphate) (cAIMP), cyclic [I(3′,5′)pI(3′,5′)p] di-inosine monophosphate (c-di-IMP), MK-1454, dimeric amidobenzimidazole (diABZI), and 5,6-dimethylxanthenone-4-acetic acid (DMXAA).

### Hydrogels

In aqueous solution, the disclosed prodrug composition can assemble into one-dimensional nanostructures, such as filamentous nanostructures, forming a nanostructure solution. In some cases, the one-dimensional nanostructures may assemble into nanotubes (NTs). Upon local in vivo administration, the nanostructure solution can immediately form a hydrogel. Thus, the disclosure also provides a hydrogel comprising the above-described prodrug composition. The term “hydrogel,” as used herein, refers to a three-dimensional network composed of hydrophilic polymers crosslinked either through covalent bonds or via physical intramolecular or intermolecular interactions. Hydrogels can absorb large amounts of water or biological fluids (up to several thousand percent), and swell readily without dissolving. The high hydrophilicity of hydrogels is primarily due to the presence of hydrophilic moieties such as carboxyl, amide, amino, and hydroxyl groups distributed along the backbone of polymeric chains. In the swollen state, hydrogels are soft and rubbery, closely resembling living tissues. Many hydrogels, such as chitosan and alginate-based hydrogels, exhibit desirable biocompatibility (see, e.g., El-Sherbiny, I. M., and Yacoub, M. H. Global Cardiology Science & Practice, 2013(3): 316-342 (2013); and Kyung et al, J. Appl. Polym. Sci., 83: 128-136 (2002)). Since their discovery more than 50 years ago, hydrogels have been employed in a variety of applications including, for example, drug delivery, wound healing, ophthalmic materials, and tissue engineering (see, e.g., El-Serbiny and Yacoub, supra; Hoffman, A. S., Ann. NY Acad. Sci., 944: 62-73 (2001); and Peppas et al., Eur. J. Pharm. Biopharm., 50: 27-46 (2000)).

Hydrogels typically reach their equilibrium swelling when a balance occurs between osmotic driving forces, which encourage the entrance of water or biological fluids into the hydrophilic hydrogel matrix, and the cohesive forces exerted by the polymer strands within the hydrogel. These cohesive forces resist the hydrogel expansion and the extent of these forces depends particularly on the hydrogel crosslinking density. Generally, the more hydrophilic the polymer forming the hydrogel, the higher the total water amount absorbed by the hydrogel. Likewise, the higher the crosslinking extent of a particular hydrogel, the lower the extent of the gel swelling. Hydrogels in their dried forms are referred to in the art as “xerogels,” while dry porous hydrogels resulting from the use of drying techniques (e.g., freeze-drying or solvent extraction) are referred to in the art as “aerogels” (see, e.g., Guenet, J. M., Thermoreversible gelation of polymers and biopolymers; Academic Press, New York (1992), p. 89).

Hydrogels can be classified based on a variety of characteristics, such as, for example origin, durability, response to stimuli, charge, structure, and composition. With respect to origin, hydrogels can be classified as natural, synthetic or semi-synthetic. Most synthetic hydrogels are synthesized by traditional polymerization of vinyl or vinyl-activated monomers. The equilibrium swelling values of these synthetic hydrogels vary widely according to the hydrophilicity of the monomers and the crosslinking density. Natural hydrogels typically are made of natural polymers including, for example, polynucleotides, polypeptides, and polysaccharides that can be obtained from a variety of sources (e.g., collagen from mammals and chitosan from shellfish exoskeletons). With respect to durability, hydrogels can be classified as durable (such as most polyacrylate-based hydrogels) or biodegradable (such as polysaccharide-based hydrogels), depending on their stability characteristics in a physiological environment. Biodegradable hydrogels have recently been developed in which degradable polymers inside the hydrogel matrices undergo chain scission to form oligomers of low molecular weight. The resulting oligomers are either eliminated by the organism or undergo further degradation. Such biodegradable hydrogels can be used in both biomedical and non-biomedical applications (see e.g., Zhu, W. and Ding, J., J. Appl Polym Sci., 99: 2375 (2006)). With respect to response to environmental stimuli, “smart” hydrogels have been developed that exhibit changes in swelling behavior, network structure, and/or mechanical characteristics in response to various environmental stimuli such as pH, temperature, light, ionic strength or electric field (see, e.g., Gutowska et al., J Control Release, 22: 95-104 (1992); Ferreira et al, Int J Pharm., 794: 169-180 (2000); and D'Emanuele, A. and Staniforth, J. N., Pharm Res., 8: 913-918 (1991)). These changes typically disappear upon removal of the stimulus and the hydrogels are restored to their original state in a reversible manner.

Supramolecular hydrogels are formed by the physical crosslinking of filamentous assemblies, derived of peptide amphiphile building units. Such supramolecular hydrogels employ responsive sol-gel phase transitioning, permitting their direct injection into the target sites with minimal non-surgical invasiveness. Recently, direct conjugation of therapeutic agents onto the peptides has been shown to convert the drug into an effective hydrogelator (e.g., a liquid vehicle which becomes a gel upon contact with body tissues or fluids), capable of creating a “self-delivery” hydrogel. In addition, the ability of hydrogels to respond the tumor microenvironment allows for a better controlled release kinetics and improved therapeutic efficacy.

Starting materials and reagents that may be used to prepare prodrug hydrogel compositions of the present invention are either available from commercial suppliers such as Aldrich Chemical Company (Milwaukee, Wis.), Bachem (Torrance, Calif.), Sigma (St. Louis, Mo.), or are prepared by methods well known to those of ordinary skill in the art (see, e.g., Fieser and Fieser's Reagents for Organic Synthesis, vols. 1-17, John Wiley and Sons, New York, N.Y., 1991; Rodd's Chemistry of Carbon Compounds, vols. 1-5 and supplements, Elsevier Science Publishers, 1989; Organic Reactions, vols. 1-40, John Wiley and Sons, New York, N.Y., 1991; March J; Advanced Organic Chemistry, 4th ed. John Wiley and Sons, New York, N.Y., 1992; and Larock: Comprehensive Organic Transformations, VCH Publishers, 1989). In most instances, amino acids and their esters or amides, and protected amino acids, are widely commercially available; and the preparation of modified amino acids and their amides or esters are extensively described in the chemical and biochemical literature and thus well-known to persons of ordinary skill in the art.

General procedures for preparing prodrug hydrogel compositions of the present invention involve initially attaching a carboxyl-terminal protected amino acid to a resin. After attachment, the resin is filtered, washed, and the protecting group on the alpha-amino group of the carboxyl-terminal amino acid is removed. The removal of this protecting group must take place, of course, without breaking the bond between that amino acid and the resin. The next amino, and if necessary, side chain protected amino acid, is then coupled to the free amino group of the amino acid on the resin. This coupling takes place by the formation of an amide bond between the free carboxyl group of the second amino acid and the amino group of the first amino acid attached to the resin. This sequence of events is repeated with successive amino acids until all amino acids are attached to the resin. Finally, the protected peptide is cleaved from the resin and the protecting groups removed to reveal the desired peptide. The cleavage techniques used to separate the peptide from the resin and to remove the protecting groups depend upon the selection of resin and protecting groups and are known to those familiar with the art of peptide synthesis.

In some embodiments, a prodrug hydrogel composition as described herein may have the following formula:

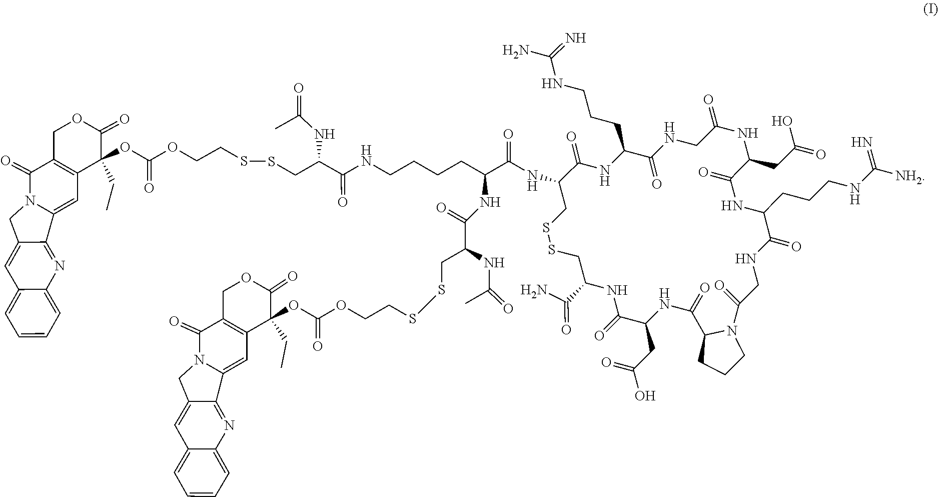

In other embodiments, a prodrug hydrogel composition as described herein may have the following formula:

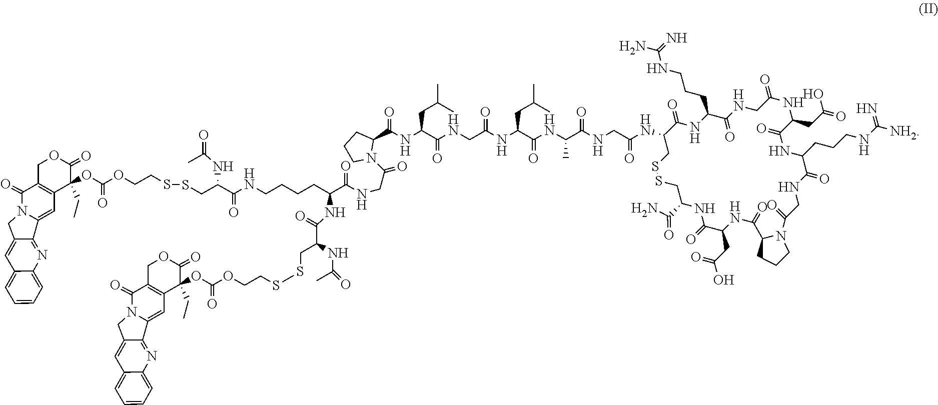

In other embodiments, a prodrug hydrogel composition as described herein may have the following formula:

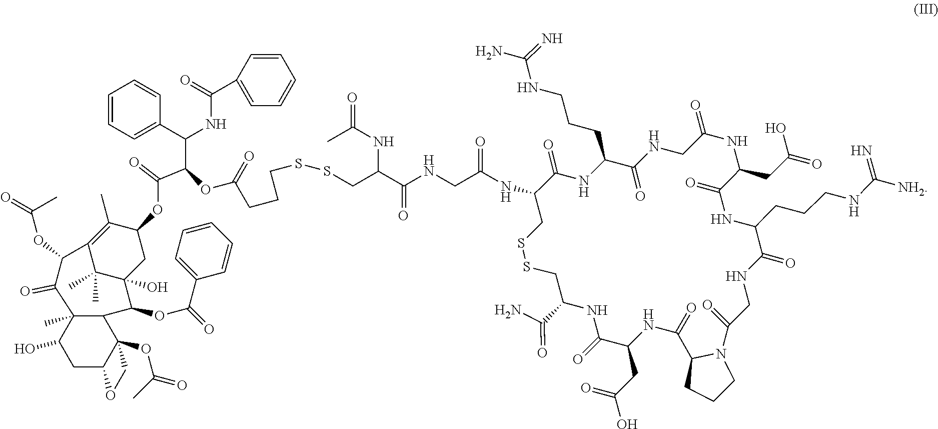

### Methods of Killing Cancer Cells

The disclosure also provides method of killing cancer cells which comprises contacting cancer cells with the above-described composition, wherein the prodrug spontaneously assembles into a hydrogel and the cytotoxic agent and immunomodulator are released from the composition, thereby killing the cancer cells.

The term “tumor,” as used herein, refers to an abnormal mass of tissue that results when cells divide more than they should or do not die when they should. In the context of the present disclosure, the term tumor may refer to tumor cells and tumor-associated stromal cells. Tumors may be benign and non-cancerous if they do not invade nearby tissue or spread to other parts of the organism. In contrast, the terms “malignant tumor,” “cancer,” and “cancer cells” may be used interchangeably herein and refer to a tumor comprising cells that divide uncontrollably and can invade nearby tissues. Cancer cells also can spread or “metastasize” to other parts of the body through the blood and lymph systems. The disclosed method ideally induces cytotoxicity in malignant tumor cells or cancer cells. The malignant tumor cells or cancer cells may be from a carcinoma (cancer arising from epithelial cells), a sarcoma (cancer arising from bone and soft tissues), a lymphoma (cancer arising from lymphocytes), a blood cancer (e.g., myeloma or leukemia), a melanoma, or brain and spinal cord tumors. The malignant tumor or cancer cells can be located in the oral cavity (e.g., the tongue and tissues of the mouth) and pharynx, the digestive system, the respiratory system, bones and joints (e.g., bony metastases), soft tissue, the skin (e.g., melanoma), breast, the genital system, the urinary system, the eye and orbit, the brain and nervous system (e.g., glioma or glioblastoma), or the endocrine system (e.g., thyroid) and is not necessarily the primary tumor. More particularly, cancers of the digestive system can affect the esophagus, stomach, small intestine, colon, rectum, anus, liver, gall bladder, and pancreas. Cancers of the respiratory system can affect the larynx, lung, and bronchus and include, for example, non-small cell lung carcinoma. Cancers of the reproductive system can affect the uterine cervix, uterine corpus, ovaries, vulva, vagina, prostate, testis, and penis. Cancers of the urinary system can affect the urinary bladder, kidney, renal pelvis, and ureter. Cancer cells also can be associated with lymphoma (e.g., Hodgkin's disease and Non-Hodgkin's lymphoma), multiple myeloma, or leukemia (e.g., acute lymphocytic leukemia, chronic lymphocytic leukemia, acute myeloid leukemia, chronic myeloid leukemia, and the like). In some embodiments, the cancer is glioblastoma or colorectal cancer.

Ideally, the disclosed method promotes inhibition of cancer cell proliferation, the eradication of cancer cells, and/or a reduction in the size of at least one tumor such that a mammal (e.g., a human) is treated for cancer. By “treatment of cancer” is meant alleviation of cancer in whole or in part. In one embodiment, the disclosed method reduces the size of a tumor at least about 20% (e.g., at least about 25%, 30%, 35%, 40%, 45%, 50%, 55%, 60%, 65%, 70%, 75%, 80%, 85%, 90%, or 95%). Ideally, the cancer is completely eliminated, i.e., the cancer cells are killed.

The cancer cells may be contacted with the composition in vitro or in vivo. The term “in vivo” refers to a method that is conducted within living organisms in their normal, intact state, while an “in vitro” method is conducted using components of an organism that have been isolated from its usual biological context. When the cell is contacted with the composition in vitro, the cell may be any suitable prokaryotic or eukaryotic cell. When the cell is contacted with the composition in vivo, the composition may be administered to an animal, such as a mammal, particularly a human, using standard administration techniques and routes. Suitable administration routes include, but are not limited to, oral, intravenous, intraperitoneal, subcutaneous, pulmonary, transdermal, intramuscular, intranasal, buccal, sublingual, or suppository administration. The composition preferably is suitable for parenteral administration. The term “parenteral,” as used herein, includes intravenous, intramuscular, subcutaneous, rectal, vaginal, and intraperitoneal administration. In other embodiments, the composition may be administered to a mammal using systemic delivery by intravenous, intramuscular, intraperitoneal, or subcutaneous injection.

In an exemplary embodiment, the compositions of the present invention can be used before or after surgical resection of a tumor in a subject. In some embodiments, the compositions are applied to the tissue margins and surrounding tissues after removal of the tumor. The tissues are then surgically closed. As used herein, the term “application” refers to the local in situ administration of the compositions of the present invention to the site of interest.

Without being held to any particular mechanism of action, the compositions of the present invention allow for the sustained release of biologically active agents (e.g., cytotoxic agents) and immunomodulators directly into the tumor site and surrounding tissues, as well as being administered post-operatively to enhance the effectiveness of the surgical treatment by local chemotherapeutic action on any remaining tumor cells which evaded surgical resection. The cytotoxic agent and immunomodulator will be released from the hydrogel through dissolution and through the biodegradation of the hydrogel and the bonds between the cytotoxic agent, linker, and hydrophilic moiety, to allow diffusion of the cytotoxic agent and one or more immunomodulators to come into contact with the surrounding tissues.

The hydrogel may be applied or administered to a subject by other means. It is contemplated that the prodrug hydrogels of the present invention can be made in gel, or liquid form, and then applied to the tissues of interest by spraying, swabbing, injection, or otherwise applying the compositions directly to the tissues or tumor site.

An advantage of the compositions and methods described herein is the fact that the use of local administration allows for high concentrations of the cytotoxic agent and one or more immunomodulators at the site of the tumor without having systemic effects in the subject.

Another advantage of the compositions and methods described herein is the ability to provide chemotherapy in a sustained release formulation, in parts of the body where there would otherwise be limited access of the biologically active agent to the site of interest. For example, the brain is well known for the blood-brain barrier preventing hydrophobic and polar molecules from entering the brain tissues. Systemic doses of chemotherapeutic agents do not typically cross the barrier without other measures or formulations which can cause systemic toxicities. However, application of the compositions of the present invention directly into the brain after tumor resection avoids this common problem.

The dose of the prodrug hydrogel compositions of the present invention also will be determined by the existence, nature, and extent of any adverse side effects that might accompany the administration of a particular composition. Preferably, the composition comprises an “effective amount” of the prodrug and the immunomodulator. As used herein, the term “effective amount” refers to the amount of a therapy (e.g., a prophylactic or therapeutic agent), which is sufficient to reduce the severity and/or duration of a disease, ameliorate one or more symptoms thereof, prevent the advancement of a disease or cause regression of a disease, or which is sufficient to result in the prevention of the development, recurrence, onset, or progression of a disease or one or more symptoms thereof, or enhance or improve the prophylactic and/or therapeutic effect(s) of another therapy (e.g., another therapeutic agent) useful for treating a disease, such as cancer.

Typically, an attending physician will decide the dosage of the pharmaceutical composition with which to treat each individual subject, taking into consideration a variety of factors, such as age, body weight, general health, diet, sex, compound to be administered, route of administration, and the severity of the condition being treated. By way of example, and not intending to limit the invention, the dose of the pharmaceutical compositions of the present invention can be about 0.001 to about 1000 mg/kg body weight of the subject being treated, from about 0.01 to about 100 mg/kg body weight, from about 0.1 mg/kg to about 10 mg/kg, and from about 0.5 mg to about 5 mg/kg body weight. In another embodiment, the dose of the pharmaceutical compositions of the present invention can be at a concentration from about 1 nM to about 10,000 nM, preferably from about 10 nM to about 5,000 nM, more preferably from about 100 nM to about 500 nM.

In some embodiments, the method induces a memory T cell response against a cancer in the subject. The term “memory T cell,” as used herein, can be defined as a CD8⁺ T cell that has responded to a cognate antigen and persists long-term. Compared to naïve cells of the same antigen-specificity, memory T cells persist in greater numbers; can populate peripheral organs; are poised to immediately proliferate, execute cytotoxic functions, and secrete effector cytokines upon antigenic re-encounter; and exist in different metabolic, transcriptional, and epigenetic states (Homann et al., Nat Med., 7: 913-9. doi: 10.1038/90950 (2001); Masopust et al., J Immunol., 172: 4875-82. doi: 10.4049/jimmuno1.172.8.4875 (2004); DiSpirito J R, Shen H, Cell Res., 20: 13-23. doi: 10.1038/cr.2009.140 (2010); Veiga-Fernandes H, Rocha B., Nat Immunol. 5: 31-7. doi: 10.1038/ni1015 (2004); and Lalvani et al., J Exp Med., 186: 859-65 (1997)). As such, hosts possessing memory T cells are often better protected against solid tumors and infection with intracellular bacteria, viruses, and protozoan parasites than their naïve counterparts (Martin, M. D., Badovinac, V. P., Frontiers in Immunology, 9: 2692 (2018)). Two major subsets of memory T cells have been identified: CD62L^{lo}/CCR7^{lo} effector memory T cells (Tem) and CD62L^{hi}/CCR7^{hi} central memory T cells (Tcm). Expression of CCR7 and CD62L on Tcm cells facilitates homing to secondary lymphoid organs, while Tem cells are more cytolytic and express integrins and chemokine receptors necessary for localization to inflamed tissues (Sallusto et al., Nature, 401: 708-12. doi: 10.1038/44385 (1999)).

The disclosed method can be performed in combination with other therapeutic methods to achieve a desired biological effect in a patient. Ideally, the disclosed method may include, or be performed in conjunction with, one or more cancer treatments. The choice of cancer treatment used in combination with the disclosed method will depend on a variety of factors, including the cancer/tumor type, stage and/or grade of the tumor or cancer, the patient's age, etc. Suitable cancer treatments that may be employed include, but are not limited, surgery, chemotherapy, radiation therapy, immunotherapy, hormone therapy, and stem cell transplantation.

The following examples further illustrate the invention but, of course, should not be construed as in any way limiting its scope.

### Examples 1-16

The following methods and materials were employed in Examples 1-16.

### Study Design

The objective of this study was to develop the in situ-formed, prodrug hydrogels for local delivery of aPD1 and CPT for enhanced cancer chemo-immunotherapy. The antitumor efficacy was evaluated in GL-261 and CT 26 tumor models. Mice were randomized to different treatment groups on the basis of tumor size and body weight. Animals from varying groups were imaged to assess tumor progression, tracked to create survival curves, and re-challenged with tumors to assess immune memory. To assess the phenotype of the TME, mice were euthanized at various time points. Sample sizes were selected on based on previous experimental experience. Body weight and tumor size were measured every two days, and mice were euthanized when tumor volume exceeded 1 cm3 or when body weight loss exceeded 20%. Blinding was not performed. All experiments were repeated at least three times.

### Materials

Rink Amide MBHA Resin and amino acids were obtained from AAPPTEC (Louisville, Ky.). Anti-PD1 antibody (cat. no. 135234) was purchased from BioLegend. Camptothecin (CPT) was purchased from Ava Chem Scientific (San Antonio, Tex.). Human MMP2 recombinant protein was obtained from Thermo Fisher. Cy3 NHS-ester and Cy5 NHS-ester were purchased from Lumiprobe. CDA (2′3′-c-di-AM(PS)2 (Rp,Rp), cat. no. tlrl-nacda2r) was purchased from InvivoGen. FITC labeled-c-di-AMP (cat. no. F011) and Cy7-labeled cGAMP (cat. no. C240) were purchased from Biolog Life Science Institute. All other chemicals were purchased from Sigma-Aldrich and were used without any further purification.

### Cell Lines

GL-261-luc brain cancer cell line, 4T1-luc breast cancer cell line, and CT 26 colon cancer cell line were generously donated by M. Lim, J. Hanes, and J. Fu at The Johns Hopkins University School of Medicine, respectively. GL-261-luc and 4T1-luc cells were grown in DMEM (Gibco, Invitrogen) supplemented with 10% FBS, 100 Units/mL penicillin G sodium, 100 μg/mL streptomycin sulfate, and 100 μg/ml of G418 (Invitrogen). CT 26 cells were grown in RPMI 1640 (Gibco, Invitrogen) supplemented with 10% FBS, 100 Units/mL penicillin G sodium, and 100 μg/mL streptomycin sulfate.

### Animals

8-10 weeks old Female C57BL/6, BALB/c mice (Charles River) and C56BL/6J-Tmem173gt/J STING-deficient mice (STINGgt/gt, Jackson Laboratory) were kept under a 12 h light/dark cycle at the Animal Care Facility with food and water ad libitum. Animal experiments were performed in accordance with the animal protocol approved by the Animal Care and Welfare Committee at The Johns Hopkins University.

Synthesis of diCPT-iRGD

The peptide C₂K-cyl[CRGDRGPDC] (SEQ ID NO: 7) was synthesized using an AAPPTEC Focus XC synthesizer via the standard Fmoc-solid phase technique. The peptide was confirmed by MALDI-TOF MS. CPT-etcSS-Pyr was synthesized using the same method as reported previously. Then, C₂K-cyl[CRGDRGPDC] (10 mg, 7.2 μmol) was dissolved in an N2-purged DMSO solution of CPT-etcSS-Pyr (16 mg, 28.8 μmol) and allowed to react overnight. The solution was purified by RP-HPLC. Product fractions were combined and immediately lyophilized to give diCPT-iRGD as a white to yellow powder. The product identity was confirmed by ESI MS.

Synthesis of diCPT-PLGLAG-iRGD.

The peptide C₂K-GPLGLAG-cyl[CRGDRGPDC] (SEQ ID NO: 8) was synthesized using an AAPPTEC Focus XC synthesizer via the standard Fmoc-solid phase technique. The purity and identity of the peptide were confirmed by HPLC and MALDI-TOF MS, respectively. CPT-etcSS-Pyr was synthesized using the same method as previously reported (Chem. Commun. 50, 6039-6042 (2014)). Then, C₂K-GPLGLAG-cyl[CRGDRGPDC] (100 mg, 51 μmol) was dissolved in 5 ml N₂-purged DMSO solution of CPT-etcSS-Pyr (115 mg, 204 μmol) and allowed to react overnight. The solution was purified by RP-HPLC and the product was lyophilized to obtain diCPT-PLGLAG-iRGD. The identity of diCPT-PLGLAG-iRGD was confirmed by ESI MS.

Self-Assembly of diCPT-PLGLAG-iRGD.

For preparation of self-assembled diCPT-PLGLAG-iRGD nanotubes (P-NT), diCPT-PLGLAG-iRGD conjugate was directly dissolved in deionized water at 500 μM and aged for 24 h at room temperature. The structure was then characterized by transmission electron microscopy (TEM, Technai12 TWIN).

### Preparation of CDA-NT

For preparation of self-assembled NTs, the diCPT-iRGD conjugate was directly dissolved in deionized water at 500 μM and aged for 24 h at room temperature. After 24 hours, adding cyclic-di-AMP molecule into nanotube solution. The negative charged cyclic-di-AMP condensed onto positive charged nanotube surface based on electrostatic interactions. The gelation was triggered to occur in physiological conditions, where biomolecules and counter ions screen charges and promote nanotube-nanotube interactions to percolate into a network. The structure was then imaged by transmission electron microscopy (TEM, Tecnai12 TWIN). CDA-NT was prepared by adding 20 μg of CDA to the diCPT-iRGD NT solution (CPT, 100 μg in 25 μl). After vortexing, the mixtures were incubated at 37° C. for 30 min. The zeta potential and morphology of CDA-NT were determined using a Zetasizer and TEM, respectively. For in vitro release study and confocal imaging, CDA was labeled with FITC. For in vivo imaging, Cy7-CDA was used.

### Hydrogel Formation Assay

Solution-to-hydrogel transition experiments were performed by adding 20 μl of 10×PBS to 180 μl of 7.2 mM P-NT solution. The sol-gel transition behavior was tested by the inverted-vial method. For preparation of aPD1 loaded hydrogel (P-NT-aPD1), 50 μg of aPD1 was added to the P-NT solution (150 μg of CPT). After vortexing, the mixture was incubated at 37° C. for 30 min. 10×PBS was then added to the P-NT-aPD1 solution to induce the formation of a hydrogel. Distribution of aPD1 in hydrogel was further characterized using confocal microscopy (Zeiss LSM 510). For in vitro release studies and confocal imaging, aPD1 was labeled with Cy3. For in vivo imaging, Cy5.5 labeled aPD1 was used. For the diCPT-iRGD NT or CDA-NT solutions, solution-to-gelation transition experiments were performed by adding 20 μl of 10×PBS to 180 μl of 5.75 mM diCPT-iRGD NT or CDA-NT solutions. The sol-gel transition behavior was tested by the inverted-vial method. Encapsulation and distribution of CDA (labeled with FITC) in hydrogel was further characterized by a confocal microscope (Zeiss LSM 510).

### GSH Responsive Drug Release

Briefly, 20 mM glutathione (GSH) and 400 μM diCPT-PLGLAG-iRGD or diCPT-iRGD stock solutions were prepared in water or PBS (pH 7.4). Equal volumes of GSH and diCPT-PLGLAG-iRGD or diCPT-iRGD solutions were then mixed to reach a final GSH concentration of 10 mM and the mixtures were incubated at 37° C. Samples were then taken at predetermined time points (0, 0.5, 1, 2, 4, 8, 12 and 24 h) and quantified using HPLC.

### MMP-2 Responsive Drug Release

4 μg/ml of MMP-2 recombinant protein and 400 μM of diCPT-PLGLAG-iRGD stock solutions were prepared in water. Equal volumes of MMP-2 and diCPT-PLGLAG-iRGD solutions were then mixed and incubated at 37° C. for the desired period of time (0, 0.5, 1, 2, 4, 8, 12 and 24 h). Samples were then quantified using HPLC.

### Cytotoxicity Assay

GL-261 cells (2.5×103 cells/well) were incubated in a 96-well plate overnight. The cells were then treated with varying concentrations of CPT or diCPT-PLGLAG-iRGD or diCPT-iRGD solutions, with untreated cells used as a control. After 48 h incubation, cell viability was tested using an MTT or WST-1 assay. The percentage of cell viability was calculated as follows: Cell Viability (%)=A570sample/A570control×100%. IC₅₀ values of the drug formulations were analyzed using Graph Pad Prism 5.

### Inhibition of Tumor Spheroids

GL-261 cells were seeded at a density of 1×10⁴ cells/well into agarose coated 48-well plates. After the culture plates were incubated at 37° C. for 7 days, the spheroids were treated with free CPT, NT and P-NT at the CPT concentration of 5 μM. The tumor spheroids were photographed every two days. Growth inhibition was evaluated by measuring the size of the tumor spheroids. Volume of the tumor spheroids was calculated using the equation V=(π×dₘₐₓ×dₘᵢₙ)/6, with dₘₐₓ dₘᵢₙ representing the maximum and minimum diameter of the spheroid, respectively.

aPD1 and CPT Release from P-NT-aPD1 Hydrogel

The in vitro hydrogel (P-NT-aPD1) release study was performed at 37° C. in the presence or absence of 2 μg/ml of MMP-2. The quantity of aPD1 (labeled with Cy3) released was determined using a Fluorescence spectrophotometer whereas CPT release was quantified using HPLC.

Hydrogel Release Studies for CDA and diCPT-iRGD

The in vitro release study was performed at 37° C. in PBS or in 10% FBS. The released CDA and diCPT-iRGD were determined using HPLC. To evaluate the in vivo release of CDA and CPT, Cy7-labeled free CDA or CDA-NT solution was injected into the tumors. At predetermined time points, fluorescence images were recorded by an IVIS Spectrum imaging system (Perkin Elmer). Mice were sacrificed to measure the amount of CPT in the tumors by HPLC. Harvested tumors were also dissected and snap-frozen in optimal cutting temperature compound (Thermo Fisher Scientific). Micrometer sections were cut using a cryotome and stained with DAPI. The slides were then imaged using a confocal microscope (Zeiss LSM 510).

### In Vivo Hydrogel Degradation

100 μl of 7.2 mM P-NT solution or diCPT-iRGD NT solution (5.75 mM, 100 μL) was injected into the back of C57BL/6 mice. The mice were then sacrificed at predetermined time points (1 hour, 5, 15, 30 and 45 days). The remaining hydrogel in each mouse was photographed and the amount of drug that persisted within the hydrogel was determined using HPLC.

In Vivo Drug Release from P-NT-aPD1 Prodrug Hydrogel

To evaluate the in vivo release of aPD1 and CPT, free (CPT+aPD1) or P-NT-aPD1 solution was injected into the tumors. Fluorescence imaging of Cy5.5-aPD1 was monitored by an IVIS Spectrum imaging system (Perkin Elmer). At predetermined time points, mice were sacrificed and major organs and tumors were collected and imaged. Bioluminescence images were analyzed using Living Image software (Perkin Elmer). The amount of CPT in the tumors was measured by HPLC. Harvested tumors were also snap-frozen, cut into micrometer sections, and stained with DAPI. The slides were then imaged using a confocal microscope (Zeiss LSM 510).

### In Vivo Tumor Models and Treatment

For the subcutaneous GL-261-luc tumor model, 2.5×10⁶ cells were inoculated on the right flanks of female C57BL/6 mice. After ten days (tumor volume had reached 100-150 mm³), mice were randomly assigned to five groups (n=5−10). Then the mice were intratumorally (it.) injected with saline (30 μl on days 10, 17 and 24), free (CPT+aPD1) ((50 μg CPT+50 μg aPD1)/30 μl on days 10, 17 and 24), diCPT-PLGLAG-iRGD NT solution (P-NT, 150 μg CPT/30 μl single injection on day 10), aPD1 loaded diC₁₂-PLGLAG-iRGD NF (aPD1(L), 50 μg aPD1/30 μl single injection on day 10), or P-NT-aPD1 ((150 μg CPT+50 μg aPD1)/30 μl single injection on day 10). For re-challenge studies, mice with long term survival from all treatment groups were inoculated with 2.5×10⁶ GL-261-luc cells on their opposite flank to develop new tumors.

To assess the systemic anti-tumor effects of P-NT-aPD1, mice were implanted with GL-261-luc cells in both the right back and left brain. Briefly, 2.5×10⁶ cells were inoculated on the right flanks of female C57BL/6 mice. The orthotopic glioblastoma tumors were established by implanting 5×10⁴ GL-261-luc cells into the left frontal lobe. The injection site was 2.5 mm lateral, 0.5 mm anterior from the bregma and 2.5 mm deep from the outer border of the cranium. Mice were imaged using an IVIS System (Perkin Elmer) to evaluate tumor growth. The tumor take rate was 100%. After six days, tumors on the right flank were treated with P-NT-aPD1 hydrogel (n=10), while orthotopic gliomas were designated as ‘distant tumors’ and did not receive any treatment. Control mice (n=5) were treated with Saline.

The tumor size and body weight were measured every 2 days. The tumor volume was calculated by the formula: V=(major axis)×(minor axis)2/2. The mice were also monitored using an IVIS Spectrum Imaging System (Perkin Elmer). Mice were euthanized when tumor volume exceeded 1000 mm³ or when body weight loss surpassed 20%.

In Vivo Tumor Models and Treatment for CDA and diCPT-iRGD.

To prepare tumor models, 2.5×10⁶ GL-261-luc cells were planted on the right flank of female C57BL/6 mice or STINGgt/gt mice and 5×10⁵ CT 26 cells were inoculated on the right flank of female BALB/C mice. After the tumor volume reached ˜100-150 mm³ (10 days for GL-261 bearing mice, 8 days for CT 26 bearing mice), tumor-bearing mice were randomly assigned to five groups (n=5-10). Then, mice were intratumorally (it) injected with either saline, free CPT+CDA, CPT NT (NT), CDA loaded diC₈-iRGD NT (CDA-L), or CDA-NT (20 μg CDA per mouse, 100 μg CPT per mouse, 25 μL), as appropriate. For the metastatic breast cancer model, 2×10⁶ 4 T1-luc cells were inoculated orthotopically into the mammary gland of female BALB/C mice to generate primary tumor. After six days, tumor-bearing mice were randomly assigned to five groups and intratumorally injected with either saline, free CPT+CDA, NT, CDA-L, or CDA-NT (20 μg CDA per mouse; 100 μg CPT per mouse, 25 μL), as appropriate. (n=6 for saline treated group, n=8 for other groups). The tumor size and body weight were measured every 3 days. The tumor volume was calculated by the formula V=(major axis)×(minor axis)²/2. The tumor was also monitored using an IVIS Spectrum Imaging System (Perkin Elmer). Mice were euthanized when the tumor volume exceeded 1 cm³ for GL-261 tumor model and 1.5 cm³ for 4T1 tumor model or when body weight loss exceeded 20%. To detect the lung metastasis of 4T1 tumor-bearing mice, mice were sacrificed on day 28. The tumors and lungs were excised, photographed, fixed and sectioned at 10 μm thickness and subjected to hematoxylin and eosin (H&E). The stained sections were imaged by microscope.

For rechallenge studies, mice with long-term survival from all groups were inoculated with 2.5×10⁶ GL-261-luc cells or 5×10⁵ CT 26 cells on the opposite flank to develop new tumors. The tumor burden was observed using an IVIS Spectrum Imaging System.

### Flow Cytometry

Tumors were minced and digested with 1 mg/mL Collagenase (Type IV, Gibco) digestion buffer. Suspensions were filtered through 70 μM cell strainers. For surface staining, cells were stained with antibodies for 30 min at 4° C. For Foxp3 staining, cells were permeabilized with Fix/Perm buffer (eBioscience) for 30 min, washed, and stained with anti-Foxp3 antibody for 30 min at 4° C. Flow cytometry was then performed using a FACSCanto II instrument (BD Biosciences). Data were analyzed with FlowJo software (Tree Star). Antibodies against CD45 (30-F11), CD4 (RM4-5 or GK1.5), CD8 (53-6.7 or 53-5.8), CD3 (17A2), CD44 (IM7), CD62L (MEL-14), Foxp3 (FJK-16s or MF-14), CDiib (ICRF44), Gr-1 (RB6-8C5), PD-1 (29F.1A12), PD-L1 (10F.9G2) NK1.1(PK136), IFN-γ(XMG1.2), KLRG1(14C2A07), MHCII(10.36), CD103(2E7) and CD69(H1.2F3) were purchased from Biolegend or eBioscience.

### In Vivo Bioluminescence and Imaging

Tumor growth in mice was observed using bioluminescence imaging. Ten minutes after intraperitoneal injection of D-luciferin (150 μg/g), mice were anesthetized and imaged using an IVIS Spectrum Imaging System (Perkin Elmer). Bioluminescence images were analyzed using Living Image software and fluorescence intensity was quantified as the average radiance (photons s⁻¹ cm sr⁻¹).

### Depletion of Immune Cells

To deplete NK cells, CD4+ T cells and CD8⁺ T cells, tumor-bearing mice were intraperitoneal injected with anti-NK1.1 (clone PK136, BioXCell), anti-CD4 (clone GK1.5, BioXCell), anti-CD8-α (clone 2.43, BioXCell) or isotype control antibody (RatIgG1, BioXcell) at an initial dose of 400 mg 1 day before treatment, followed by 200 mg every 3 days. Depletions of CD8+ T cells, CD4+ T cells and NK cells were confirmed by flow cytometric analysis of PBMCs.

### Ex Vivo Restimulation Assay

Splenocytes of 4T1-tumor bearing mice were assayed for tumor peptide-specific CD8+ T cell response. 10 days after intratumoral injection of saline, free CPT+CDA, NT, CDA-L, or CDA-NT, spleens were harvested, dissociated and treated with ACK Lysis buffer to lyse red blood cells. 1×10⁸ splenocytes were stimulated 6 hours with 1 mM AH1 peptide (SPSYVYHQF, gp70 423-431 (SEQ ID NO: 9)). Cells were then stained for intracellular IFNγ and measured on FACSCanto II instrument (BD Biosciences).

### Cytokine Detection

Tumor tissue was harvested and then homogenized in cold PBS buffer in the presence of digestive enzymes, forming single cell suspensions. The tumoral levels of IFN-α, IFNβ and CXCL10 were measured with ELISA kits (Invitrogen) according to the manufacturer's instructions.

### Statistical Analysis

Statistical analysis was performed using GraphPad Prism software 5. Data are presented as means±SD. The two-tailed unpaired t-test was used to determine statistical significance between two treatment groups. Survival was plotted using Kaplan-Meier curve and assessed by log-rank (Mantel-Cox) test. *P≤0.05, **P≤0.01, ***P≤0.001.

### Example 1

This example describes characterization of a self-assembling prodrug hydrogel.

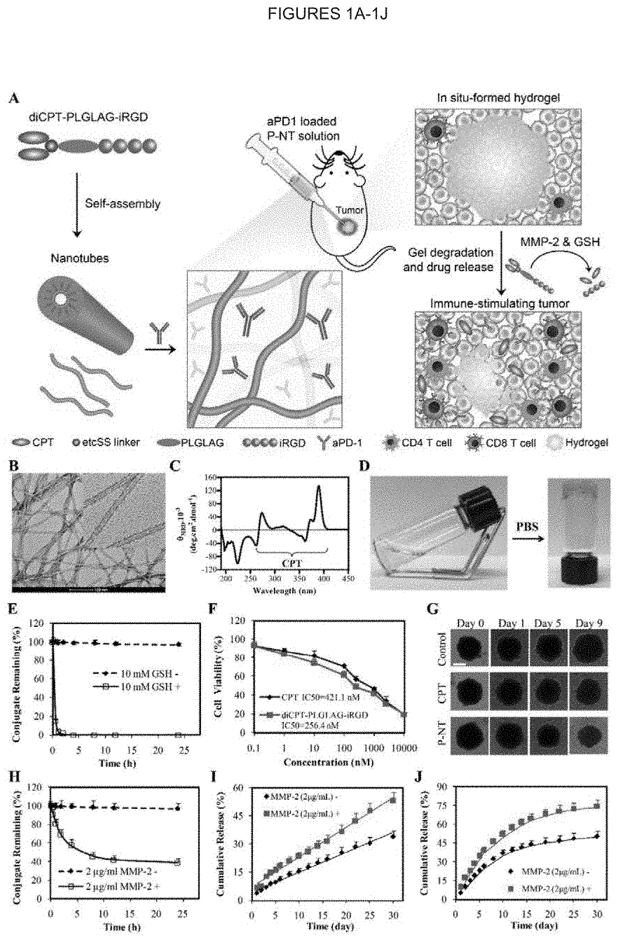

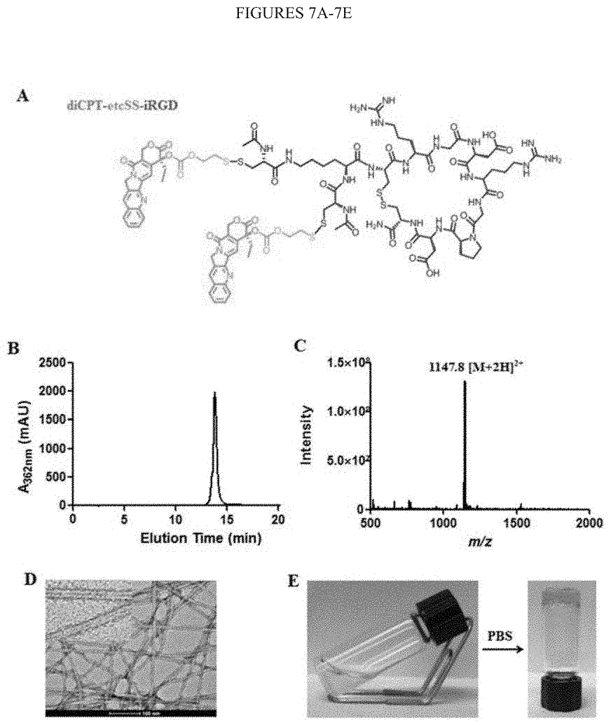

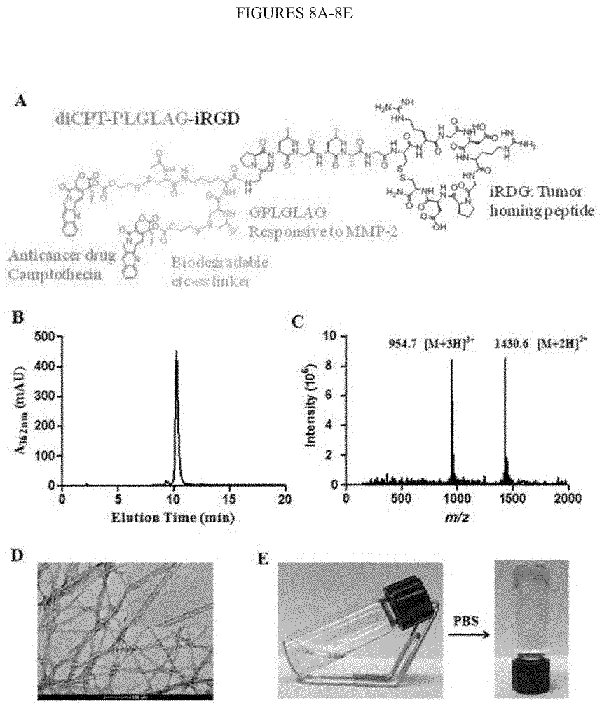

The amphiphilic prodrug, diCPT-PLGLAG-iRGD, was first synthesized by conjugating a hydrophilic iRGD (a cyclic peptide known to facilitate tumor tissue penetration of anticancer agents) to two hydrophobic CPT molecules through a matrix metalloproteinase 2 (MMP-2) responsive linker (PLGLAG (SEQ ID NO: 4) peptide) (FIGS. 7A-C). The two CPT moieties were attached to the PLGLAG (SEQ ID NO: 4) peptide through a reducible etcSS linker that forms disulfide bonds with the cysteine residues of the peptide sequence. This CPT prodrug spontaneously assembles into supramolecular nanotubes (P-NT) tens of micrometers in length in aqueous environments (FIG. 1B). Corresponding circular dichroism (CD) data showed a negative peak around 222 nm, indicative of forming highly ordered intermolecular hydrogen bonding, and a series of absorptions between 260 nm-400 nm that are attributed to CPT packed in a highly ordered fashion (FIG. 1C). It was found that increasing the concentration of the P-NT solution or adding either PBS, or cell medium could induce immediate formation of a self-supporting hydrogel (FIG. 1D). This quick sol-gel transition suggests that this CPT prodrug hydrogel can be used to deliver aPD1 by simply mixing the antibodies with the P-NTs in an admixture prior to gelation (FIG. 1A). To prepare aPD1 encapsulated P-NT hydrogel, a therapeutic dose of aPD1 (50 μg) was mixed with a P-NT solution (150 μg of CPT in 30 μl H2O). A hydrogel was resulted immediately after the addition of PBS to the mixture (FIG. 8A). Confocal imaging with Cy3-labled aPD1 suggests spatially uniform distribution of aPD1 across the hydrogel (FIGS. 8B and C).

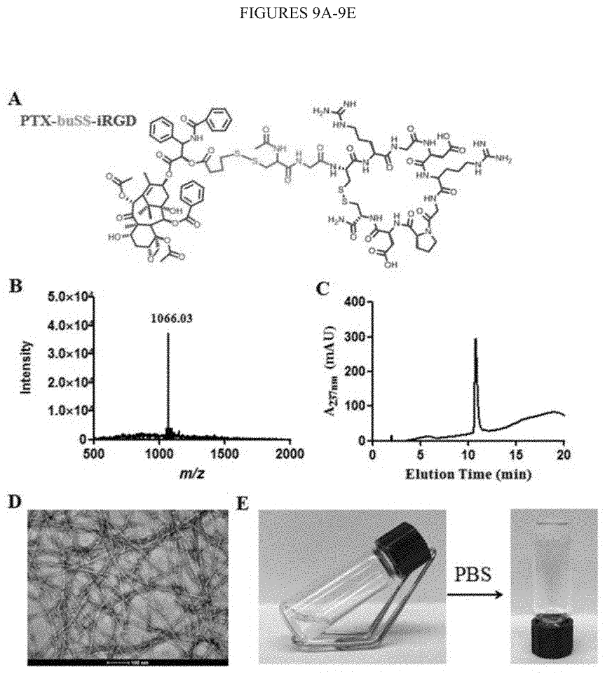

To determine the release of free CPT from the diCPT-PLGLAG-iRGD prodrug, 10 mM glutathione (GSH) was used to mimic intracellular reductive conditions. GSH is a cancer-relevant reducing agent that breaks the etcSS disulfide linker to release the parent CPT (FIG. 9). It was found that GSH-induced cleavage of diCPT-PLGLAG-iRGD occurred rapidly, with 95% of CPT prodrug degraded within 1 hour (FIG. 1E), suggesting the diCPT-PLGLAG-iRGD prodrug can quickly supply bioactive CPT in intracellular GSH reductive environment. In the absence of GSH, the drug conjugate concentration decreased only 4% over 24 hours in the absence of GSH as a result of hydrolysis. In addition, the CPT prodrug and P-NT hydrogel exhibited superior cytotoxicity against GL-261 brain tumor cells and its 3D tumor spheroids, respectively (FIGS. 1F and 1G).

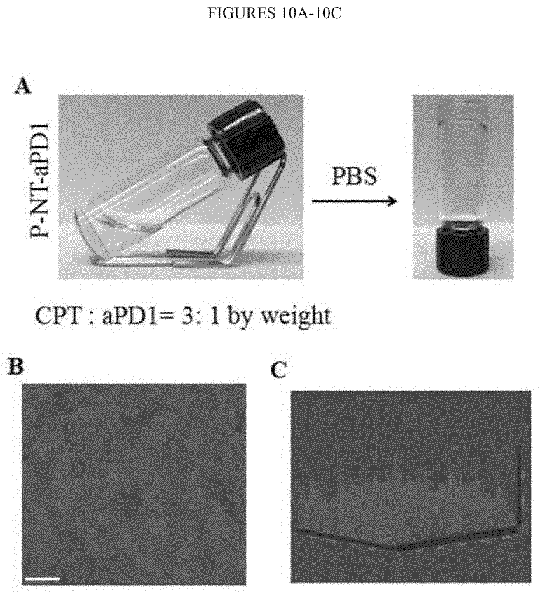

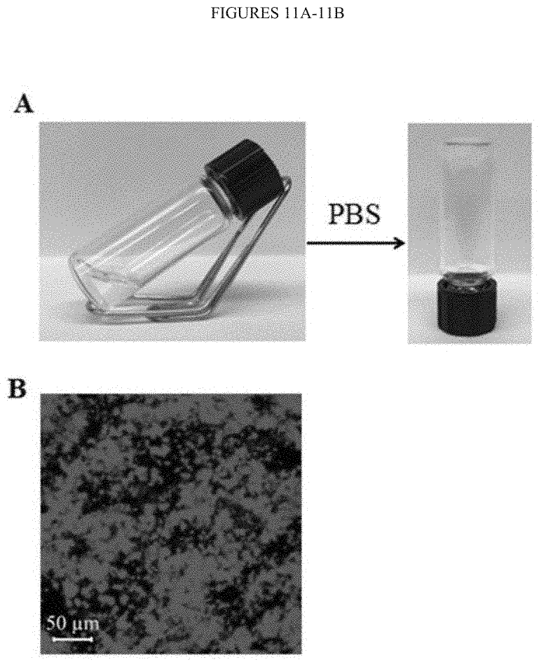

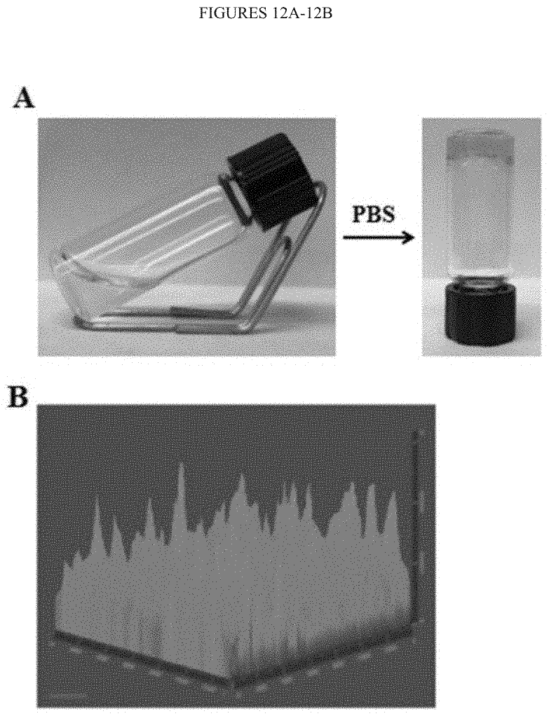

The PLGLAG peptide was used as a bioresponsive linker in some of the CPT prodrug embodiments because it is cleavable by MMP-2, an enzyme known to be overexpressed in the tumor extracellular matrix. MMP-2 was selected as the cleavage enzyme due to its association with the progression of malignant tumors and high expression in various tumor types including GL-261 brain tumors (FIG. 10). To test the cleavability of the PLGLAG spacer, diCPT-PLGLAG-iRGD was incubated with the MMP-2 enzyme and the resultant products were analyzed using HPLC and MS. It is apparent that a new peak, identified as the cleaved product LAG-iRGD (FIG. 11), was eluded at a retention time of 2.9 min. Monitoring the changes of this peak over time shows that diCPT-PLGLAG-iRGD is quickly degraded by MMP-2, with 54% cleaved after 8 hours (FIG. 1H). TEM and CD (FIG. S6) studies confirmed further MMP-2 accelerates the degradation of the P-NT hydrogel by cutting and shortening these supramolecular nanotubes. The release of CPT and aPD1 from the P-NT-aPD1 hydrogel was subsequently investigated in the presence of MMP-2. As expected, an accelerated release was observed in MMP-2 treated hydrogels. CPT prodrugs displayed a sustained, linear release from the P-NT hydrogel, (FIG. 1I), whereas aPD1 showed a much faster release rate, with 53% released within 10 days in the presence of MMP-2 (FIG. 1J).

### Example 2

This example demonstrates that the supramolecular hydrogel scaffold formed from the self-assembling prodrug hydrogels extends local retention and release of aPD1 antibody.

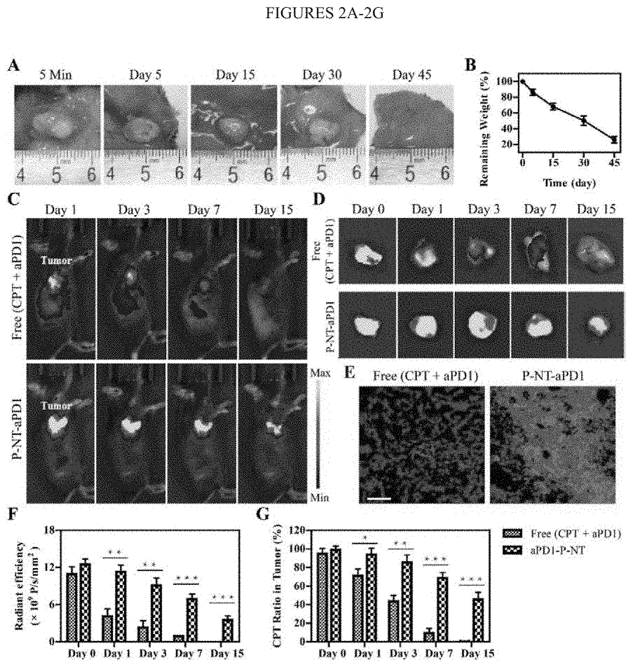

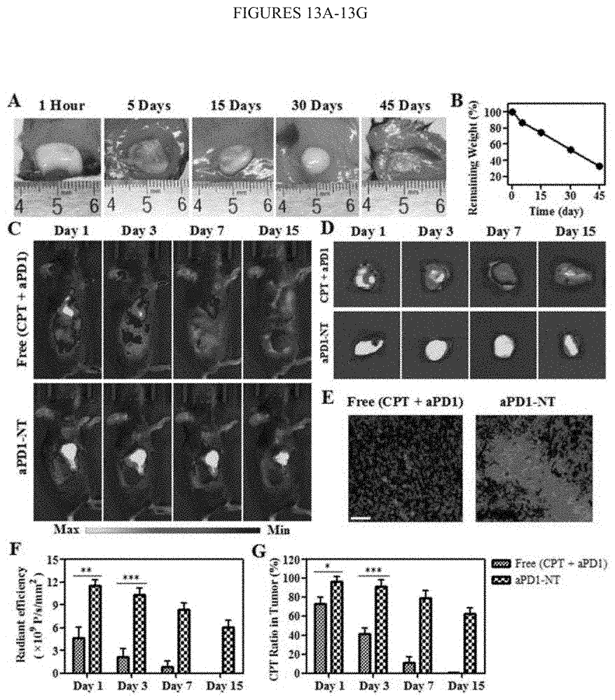

The in vivo formation of gels after injection and its subsequent degradation were then evaluated in C57BL/6 mice. After subcutaneous injection into the backs of mice, a solution containing 7.2 mM diCPT-PLGLAG-iRGD was observed to undergo a sol-gel transition within 5 minutes (FIG. 2A). This in situ formed P-NT hydrogel displayed a nearly linear degradation profile, as determined by the mass loss method, with ˜78% degraded within 45 days (FIG. 2B). To assess the potential of this prodrug hydrogel to act as a drug delivery scaffold, tumoral injections of aPD1 solutions and aPD1 encapsulating P-NT hydrogels were compared. Three days following injection of free (CPT+aPD1), it was found that Cy5.5-labeled aPD1 fluorescence appeared throughout the whole body and in major organs (FIG. 2C, FIGS. 13A and B). After seven days, the fluorescence signal were not observed in tumors, indicating that aPD1 rapidly leaked out of the tumor tissue (FIG. 2D). In stark contrast, for P-NT-aPD1 treated mice, almost all aPD1 are contained within the tumor tissue and no fluorescence was detected in other parts of the bodies (FIGS. 2C and D, FIGS. 13A and B). These results indicate that the P-NT hydrogel increases local retention of the therapeutic agents within the tumor tissue, reducing drug leakage and off-site accumulation. Furthermore, IVIS imaging revealed no fluorescence at the periphery of the tumors treated with free (CPT+aPD1) (FIG. 2D), whereas P-NT-aPD1 treated tumors displayed strong fluorescence throughout the whole tumor. These results were also confirmed by confocal imaging of tumor tissue sections (FIG. 2E and FIG. 13C). The release profiles of aPD1 and CPT were quantitatively assessed using IVIS and HPLC, respectively. For free drug treated mice, over 90% of fluorescence was lost within seven days after injection (FIG. 2F), while for P-NT-aPD1 treated mice, a strong fluorescence signal can still be detected 15 days after injection, with approximately 29.2% remaining in the tumor tissue. Consistently, CPT exhibited a steady release profile in the P-NT-aPD1 treated group, with 53.6% liberated within 15 days, in contrast to a rapid release in the free drug treated group, where no drug was detectable in the tumor site after 15 days (FIG. 2G). These results suggest that local delivery of a self-assembling prodrug, such as P-NT-aPD1, can significantly enhance tumoral drug retention, enabling a sustained release of both therapeutics over an extended period of time.

### Example 3

This example demonstrates that a P-NT-aPD1 prodrug hydrogel elicits a robust antitumor immunity.

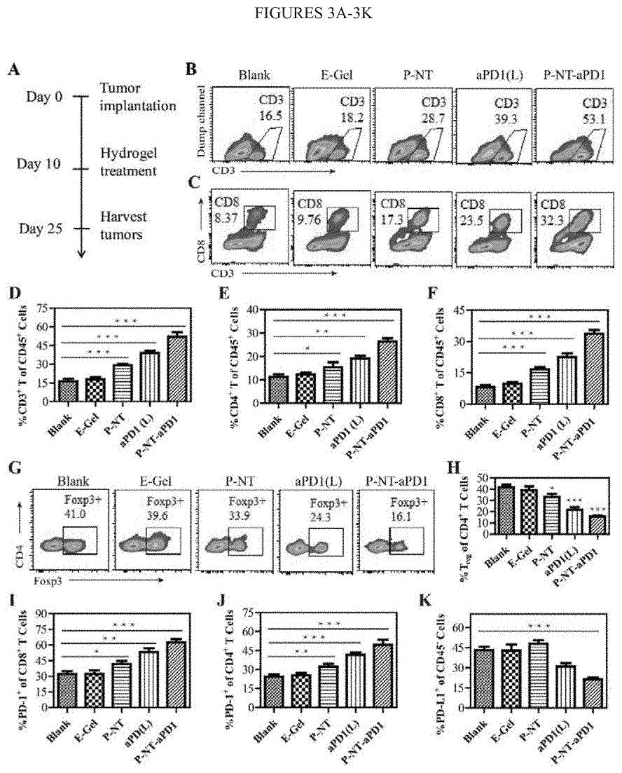

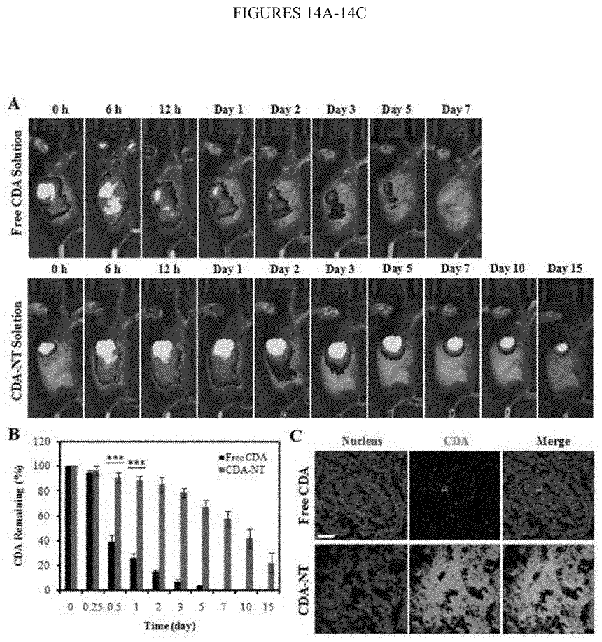

To assess the immune response of each treatment, all mice were sacrificed at day 25 post tumor implantation, and tumor infiltrating lymphocytes and tumor cells were then analyzed using flow cytometry (FIG. 3A). DiC12-PLGLAG-iRGD was designed and used as a therapeutic-free hydrogel (FIG. 14). These results suggest that this “empty” hydrogel (E-Gel) had no important effects on TILs and tumor cells (FIG. 3A), while the P-NT prodrug hydrogel increased in the proportion of CD3⁺, CD4+ and CD8+ T cells in the tumor tissue (FIG. 3B-F). This observation clearly shows that the P-NT hydrogel alone (no aPD1) could induce infiltration of T cells. When aPD1 was loaded into the E-Gel (aPD1(L) (no CPT), an increased percentage of T cells were also observed within the tumor. This suggests that as expected, the aPD1 alone can block the interactions between PD-1 and its ligands, resulting in increased T cell survival. Notably, the addition of aPD1 to P-NT (P-NT-aPD1) increased the percentage of CD8+ effector T cells in tumors by 4.1-fold and 1.5-fold, relative to that of untreated and aPD1(L) treated mice, respectively. Furthermore, P-NT treatment reduced moderately the percentage of FoxP3+CD4+ regulatory T cells (Tregs) and myeloid-derived suppressor cells (MDSCs, Gr-1+CD11b+CD45⁺ cells) compared to the E-Gel (FIGS. 3G, 3H and FIG. 15), whereas the combined P-NT-aPD1 treatment significantly decreased the ratio of Tregs and MDSCs. It was also found that both P-NT and aPD1(L) treatment increased the frequency of PD1+CD8+ T cells in the tumor microenvironment compared to those untreated mice (FIG. 31). The PD1+CD8+ T cell response was most evident in tumors treated with dual P-NT-aPD1 therapy. Similar effects were also observed in CD4+ T cells (FIG. 3J). Thus, the combined P-NT-aPD1 treatment provoked a potent antitumor immune response, as evidenced by the significantly decreased percentage of PD-L1+ cancer cells in P-NT-aPD1 treated tumors (FIG. 3K). These results led to the conclusion that P-NT-aPD1 treatment elicits an immune-stimulating tumor microenvironment in mice.

### Example 4

This example demonstrates that the P-NT-aPD1 prodrug hydrogel elicits complete regression of established GL-261 brain tumors.

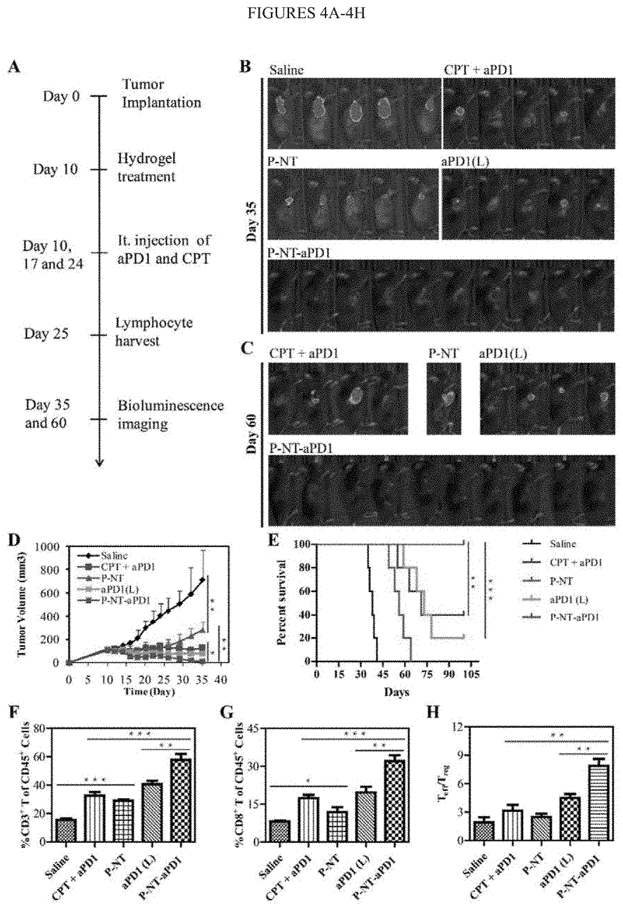

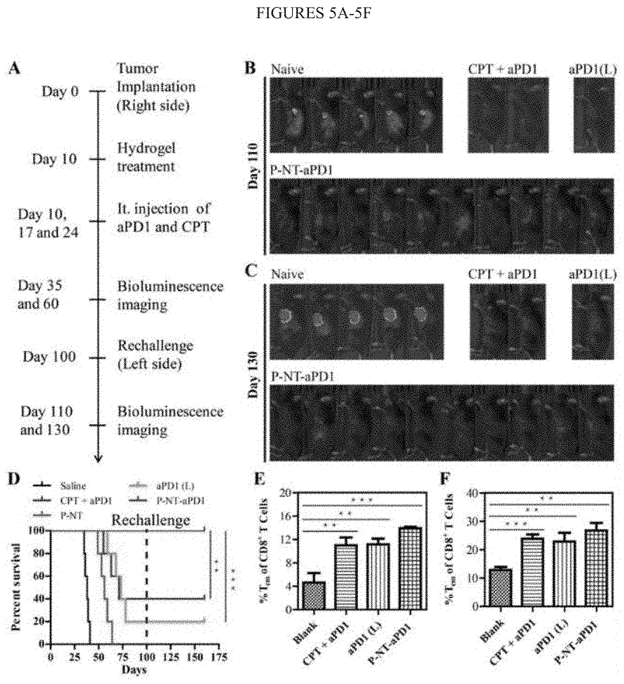

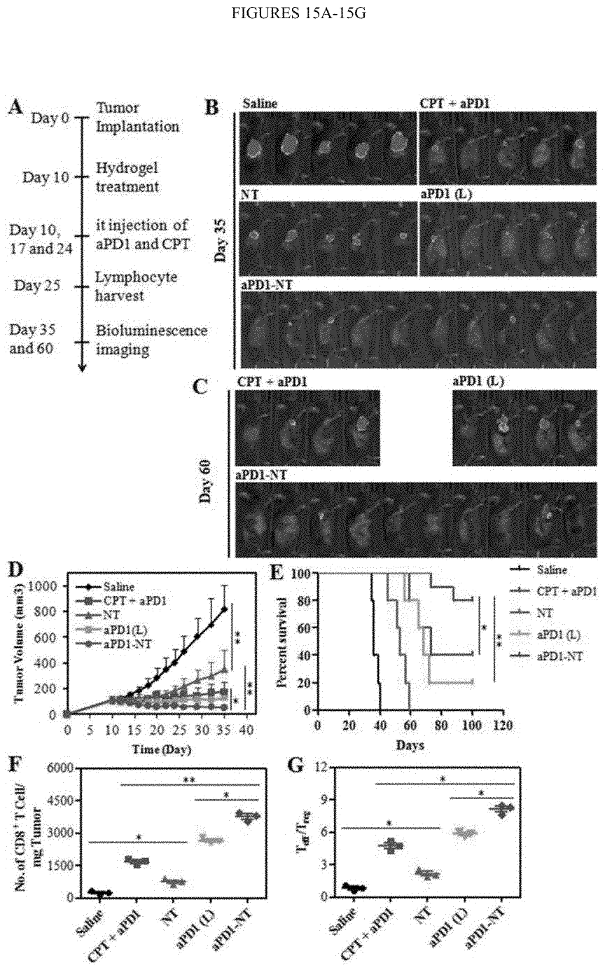

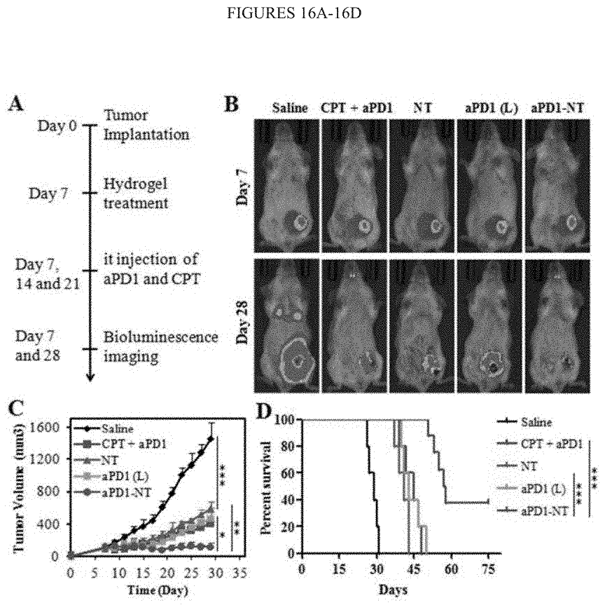

To evaluate the synergistic antitumor effects of the P-NT-aPD1 hydrogel, a subcutaneous GL-261 brain tumor model was used (FIG. 4A). Tumor burden was monitored and quantified using bioluminescence signals and caliper measurements. The in situ formed gels were injected into the tumor when its volume reached ˜100-150 mm3 at day 10. The “empty” hydrogel (E-Gel) had no tumor inhibition effect (FIGS. 16A and B). P-NT treated mice showed a delay in tumor growth (FIGS. 4B and 4C). Although aPD1(L) monotherapy was not sufficient to control tumor burden, tumor suppression was more pronounced in aPD1(L) treated mice than those treated with P-NT. Importantly, P-NT-aPD1 combination therapy resulted in the most effective tumor recession (FIGS. 4C and 5D), with all P-NT-aPD1 treated tumors fully regressed and 100% mouse survival at 100 days (FIG. 4E). In contrast, free (CPT+aPD1) treatment exhibited worse tumor growth inhibition than P-NT-aPD1 treatment, even when a much higher dose of aPD1 (150 μg vs 50 μg in P-NT-aPD1) was given over three administrations. This study confirms that the P-NT-aPD1 hydrogel has an enhanced synergistic antitumor effect. In addition, mouse body weight, blood cell count and serum biochemistry showed no significant difference following P-NT-aPD1 treatment, indicating that local delivery of P-NT-aPD1 did not induce obvious side effects (FIG. 16C, table S1). Residual tumors were collected on day 25 post tumor implantation and cells were subsequently analyzed using flow cytometry to investigate immune cell subset changes in response to different treatments. P-NT-aPD1 treated mice exhibited the highest frequencies of CD3⁺, CD4+, and CD8⁺ T cells among all the treatment groups (FIGS. 4F, 4G and FIG. 16D). The percentage of CD8⁺ T effector cells in the P-NT-aPD1 treated mice was 1.6-fold higher than the aPD1(L) treated group and 1.9-fold higher than that of the free (CPT+aPD1) treated mice. Moreover, the ratio of tumor-infiltrating CD8⁺ T cells (T_{eff}) to T_{reg} was substantially increased post P-NT-aPD1 treatment (FIG. 4H). Collectively, these results suggest that P-NT-aPD1 can effectively suppress tumor growth in mice by eliciting a robust, T cell-mediated, antitumor immune response.

### Example 5

This example demonstrates that the P-NT-aPD1 prodrug hydrogel induces durable immune response.

To assess whether P-NT-aPD1 therapy could induce a memory response, mice that displayed long-term survival from all previous treatment groups were re-challenged with GL-261 cells on the opposite flank on day 100, with naive mice used as controls (FIG. 5A). No tumor growth was observed in the free (CPT+aPD1), aPD1 (L), and P-NT-aPD1 groups, as evidenced by the absence of bioluminescence signal of GL-261 cells (FIGS. 5B and 5C). In contrast, all naive mice developed large tumors and died within 40 days post cancer cell implantation (FIG. 5C and FIG. 16E). These results reveal that a memory response upon tumor recognition occurred in all survived mice treated with aPD1. It is noteworthy that the 100% overall survival of the P-NT-aPD1 treated mice was significantly higher than that of any other treatment group (FIG. 5D). 60 days after tumor re-challenge, spleens were collected to analyze memory immune cells. Indeed, the percentages of CD8+CD44highCD62Lhigh central memory T (Tcm) cells and CD8+CD44highCD62Llow effector memory T (Tem) cells were both generated in the long-term survival mice (FIGS. 5E and 5F). Notably, P-NT-aPD1 treated mice showed increased frequency of CD8⁺ Tcm and Tem cells compared to other rechallenged treatment groups and the control group. These results clearly show that a durable and robust T cell memory response was generated by local delivery of P-NT-aPD1 hydrogel.

### Example 6

This example demonstrates that systemic antitumor immunity was induced by localized P-NT-aPD1 therapy.

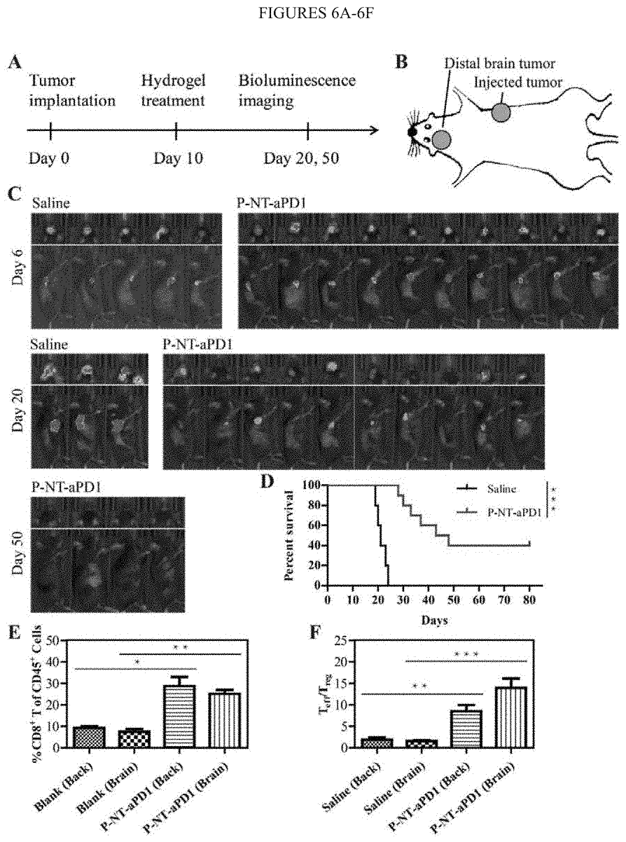

Localized P-NT-aPD1 therapy was tested to determine whether it induces systemic antitumor immunity against GL-261 tumors. An orthotopic glioblastoma tumor model was established in the left cortical surface to mimic tumor metastasis. On day 6, the primary tumors were locally treated with P-NT-aPD1 prodrug hydrogels (FIGS. 6A and B). It was observed that not only was the primary tumor growth significantly inhibited by the P-NT-aPD1 therapy, but distant intracranial glioma development was also suppressed (FIG. 6C). In contrast, all untreated mice died within 25 days as a result of orthotopic glioma (FIG. 16F). The P-NT-aPD1 therapy significantly prolonged survival time and completely eliminated tumors in 40% of mice (FIG. 6D), indicating that localized P-NT-aPD1 therapy effectively suppresses the progression of both primary and distant tumors. Consistent with these results, the frequency of CD8+ T cells and the ratio of tumor infiltrating T_{eff} to T_{reg} increased in tumors treated with P-NT-aPD1 (FIGS. 6E and 6F). These treatment outcomes suggest that localized P-NT-aPD1 therapy induced a protective systemic immune response against GL-261 tumors.

### Example 7

This example describes the efficacy of the P-NT-aPD1 prodrug hydrogel in a colon cancer model.

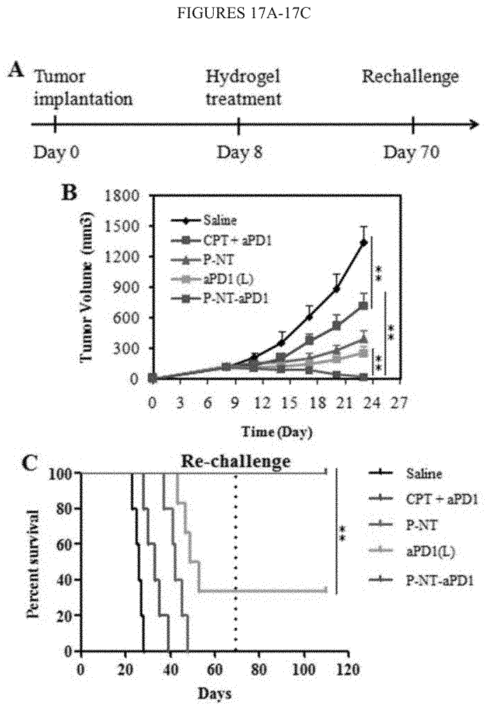

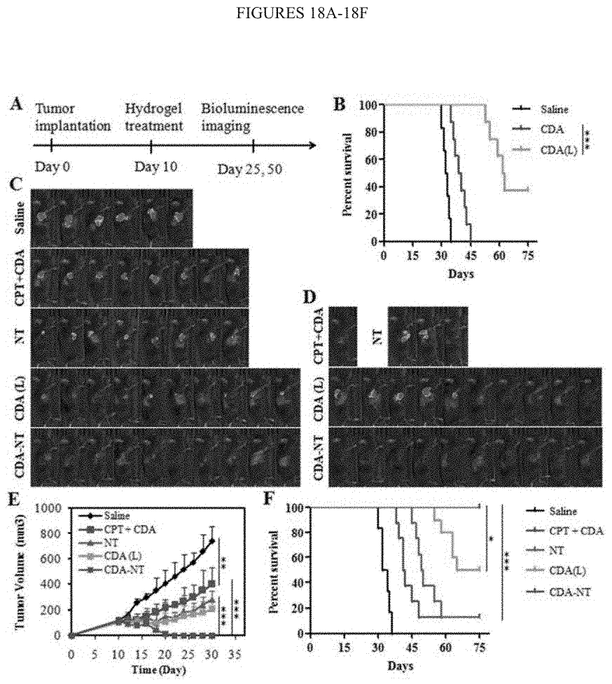

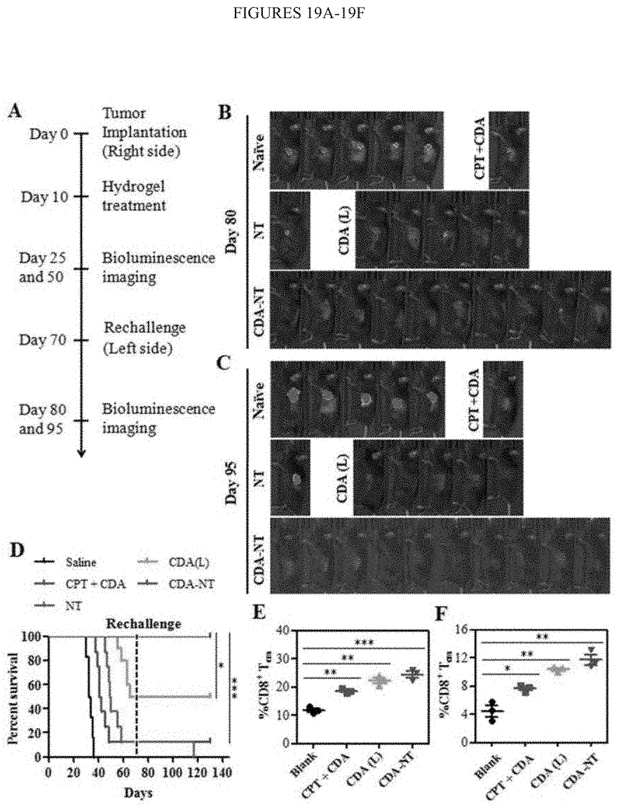

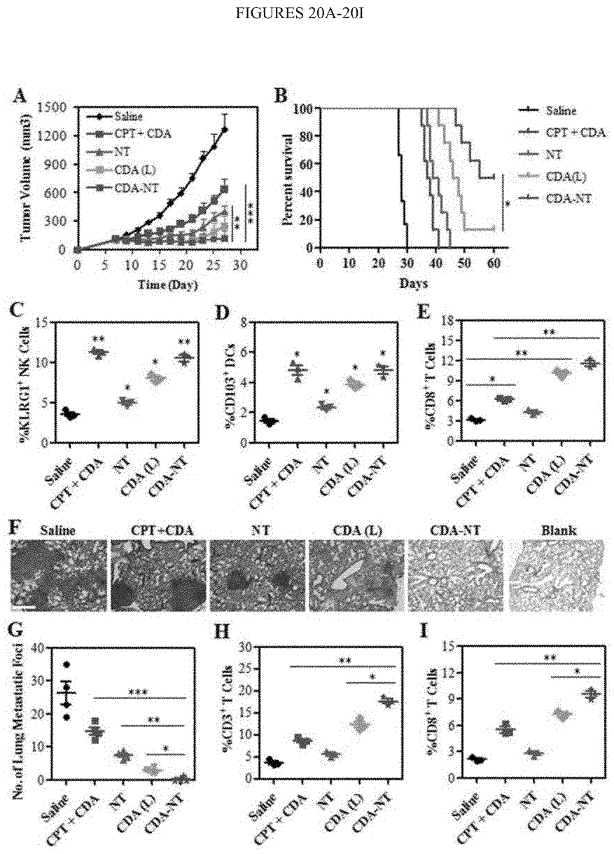

To ascertain the broad application potential of the P-NT-aPD1 prodrug hydrogel, CT 26 colon cancer bearing BALB/c mice were evaluated. When tumor volume reached 100-150 mm³ at day 8, a solution containing P-NT and aPD1 was injected into the mice to from P-NT-aPD1 hydrogel in situ (FIG. 17A). Consistent with the findings in the brain tumor model, mice treated with P-NT-aPD1 showed noticeable tumor growth inhibition (FIG. 17B). About 100% of the mice treated with P-NT-aPD1 survived for at least 130 days and developed antitumor immune memory (FIG. 17C). These results corroborate the high antitumor efficacy of P-NT-aPD1 hydrogel in cancer immunotherapy.

### Example 8

This example describes the design of an alternative in situ formed chemo-immunotherapeutic hydrogel.

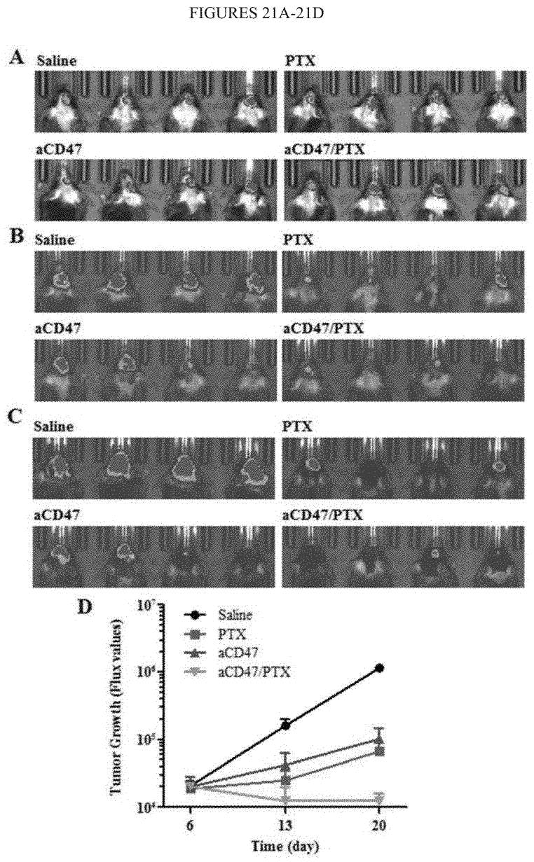

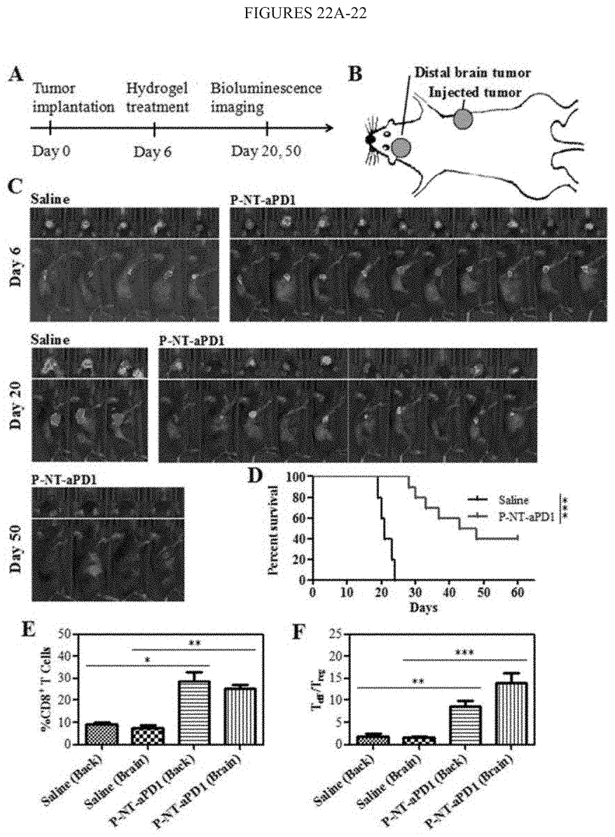

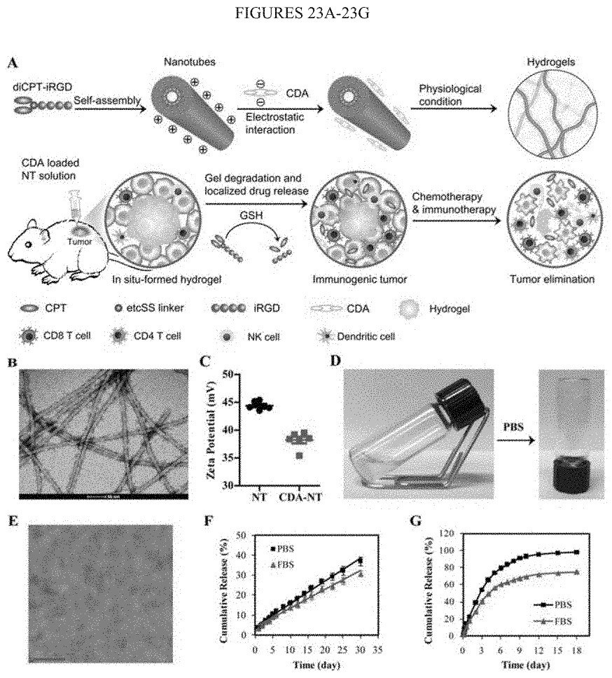

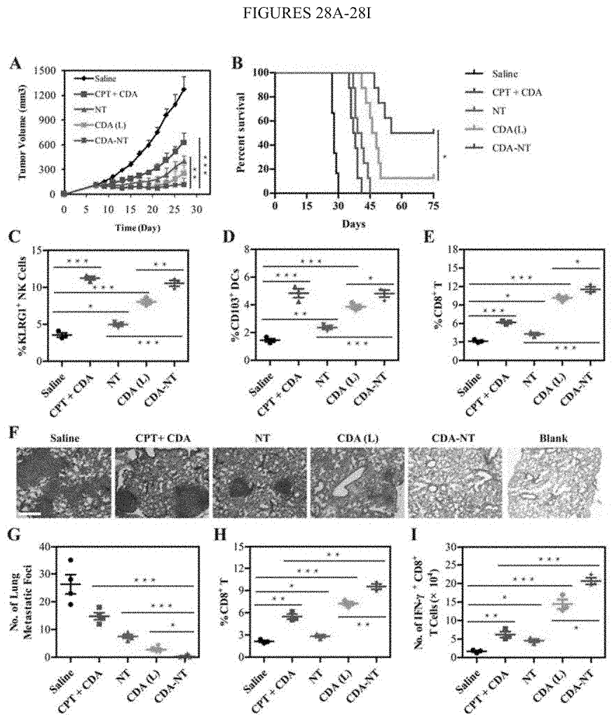

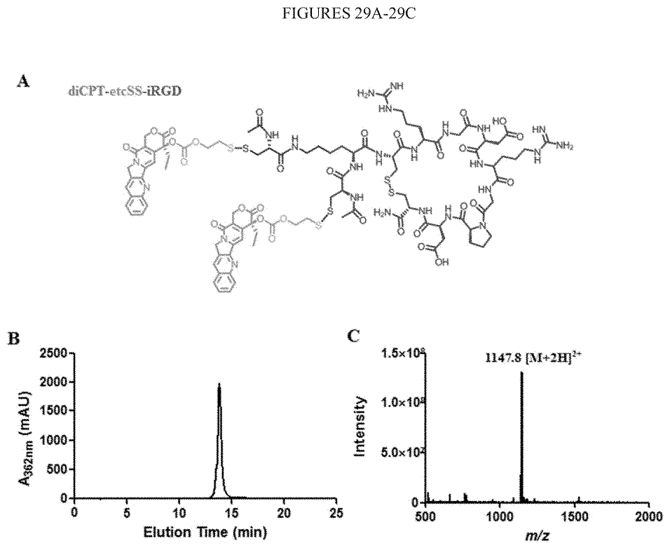

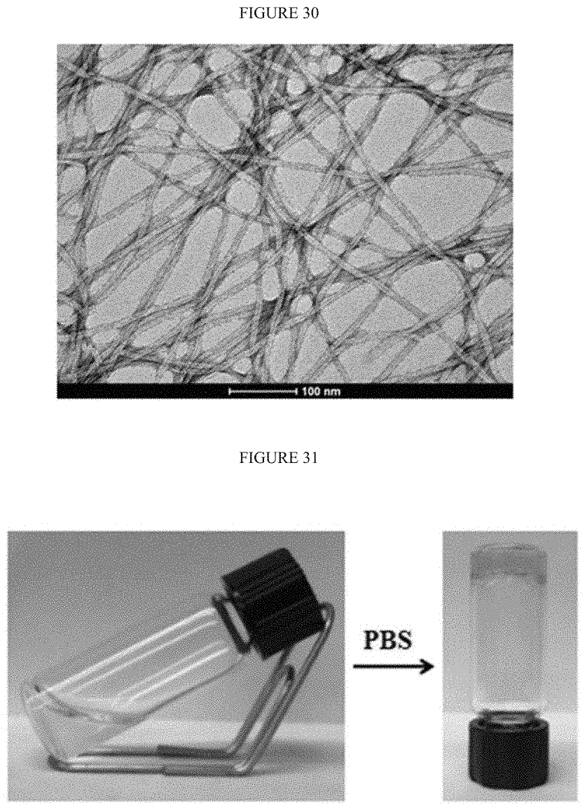

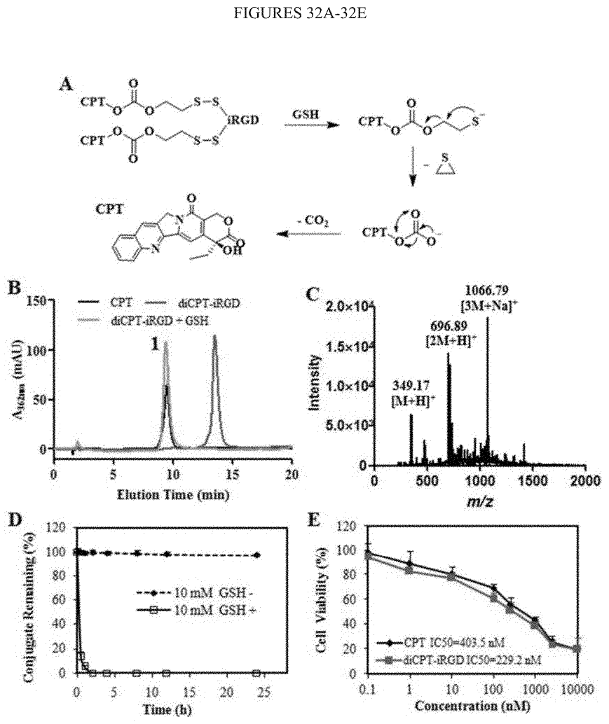

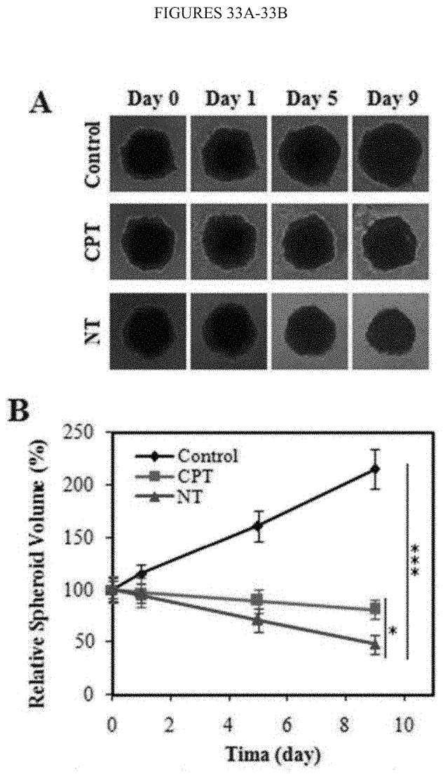

Simplicity and efficacy are the two primary considerations in the design of the self-assembling CPT drug amphiphile. Two CPT molecules were chemically conjugated to iRGD, a short peptide known to facilitate tumor tissue penetration, through a reducible disulfanyl-ethyl carbonate (etcSS) linker to form an amphiphilic molecular construct (diCPT-iRGD) (FIG. 29). This drug amphiphile spontaneously assembles into supramolecular NTs (FIG. 23B and FIG. 30) which can subsequently entangle to form a self-supporting supramolecular hydrogel under physiological conditions (FIG. 31). To probe the release of free CPT from the diCPT-iRGD drug amphiphile, a mechanistic study of glutathione (GSH)-induced degradation was performed. GSH is a cancer-relevant reducing agent that breaks the designed etcSS disulfide linker to release CPT. These results reveal that parent CPT could be effectively liberated from diCPT-iRGD in the presence of GSH, and the cleavage of diCPT-iRGD occurred rather rapidly, with nearly 100% of free CPT released within 2 hours (FIG. 32A-D). As expected, the CPT prodrug and NT hydrogel showed potent cytotoxicity against GL-261 brain tumor cells and 3D tumor spheroids (FIGS. 32E and 33).

### Example 9

To investigate the gelation and drug release behavior of the CDA containing hydrogel, a therapeutic dose of CDA (20 μg) was added into a 25 μl aqueous solution of diCPT-iRGD (100 μg equivalent of CPT). After mixing, the reduced Zeta potential suggests that the negatively charged CDAs were effectively condensed on the surface of these positively charged NTs (FIG. 23C and FIG. 34). It was further discovered that increasing the concentration of the CDA-NT or adding counterions (such as PBS or cell medium) can lead to the formation of self-supporting CDA-loaded NT hydrogels (FIG. 23D). To confirm the distribution of CDA within hydrogels, fluorescein (FITC)-labeled CDA was used to prepare the hydrogel. Confocal imaging reveals that the FITC signals of the labeled CDA were uniformly distributed across the frozen section of the hydrogel (FIG. 23E). The release behavior of diCPT-iRGD and CDA from the CDA-NT hydrogel was assessed under sink conditions in PBS (pH 7.4) and also in 10% FBS at 37° C. The diCPT-iRGD conjugate displayed a sustained and linear release from the NT hydrogel, with 37.2% of the drug released over 30 days in PBS (FIG. 23F). Notably, CDA exhibited a relatively faster release profile, with 98% released within 18 days in PBS (FIG. 23G). The non-linear release of CDA is typical of encapsulated therapeutic agents within hydrogels, showing an initial burst release, followed by a gradually reduced release rate over time. There was also a slight difference in drug release rate between in PBS and in 10% FBS, with a relatively slower rate observed in the latter. This is likely due to the presence of biomolecules in FBS that stabilize the supramolecular CPT NTs and enhance their interactions to form a mechanically stronger hydrogel, leading to retarded release of both CDA and diCPT-iRGD. Despite these variations, it is clear that a sustainable, long-term release profile was achieved in all cases for both therapeutic agents.

### Example 10

This example demonstrates that a biodegradable NT hydrogel enables local retention and sustainable release of STING agonists in vivo.

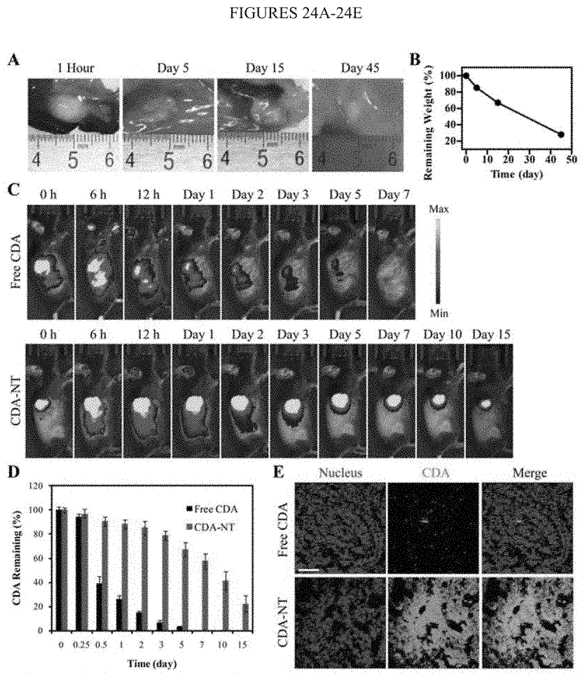

Next, the in vivo gelation and biodegradation of the diCPT-iRGD hydrogel was evaluated by subcutaneous injection of the diCPT-iRGD NT solution into the back of C57BL/6 mice. One hour after injection, the mouse was sacrificed and it was observed that the injected NT solution had translated into a well-defined, spherical-shaped hydrogel. This in situ formed NT hydrogel showed gradual biodegradation over a period of 45 days (FIG. 24A). To determine the extent of degradation over time, the amount of the remaining hydrogel was determined using HPLC. The hydrogel exhibited a near linear degradation profile, with approximately 75% degraded within 45 days (FIG. 24B). These results are consistent with the in vitro findings, confirming that the NT solution can quickly form a hydrogel after in vivo injection, and that this in situ formed hydrogel could serve as a therapeutic reservoir for sustained release of CPT in vivo.

### Example 11

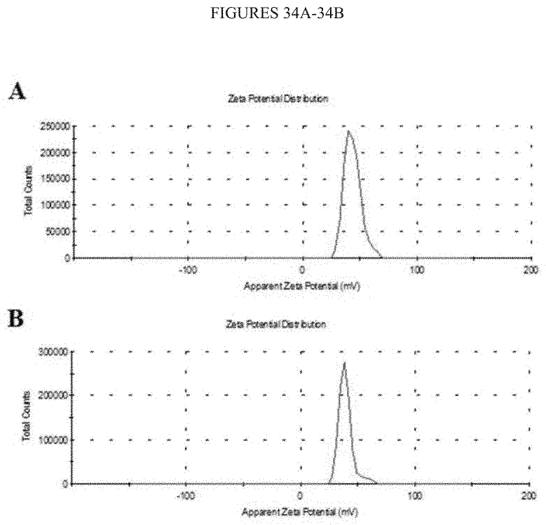

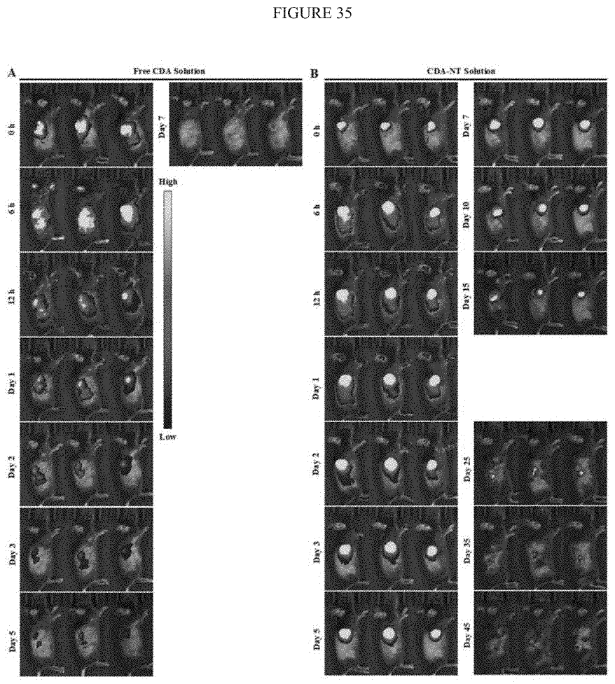

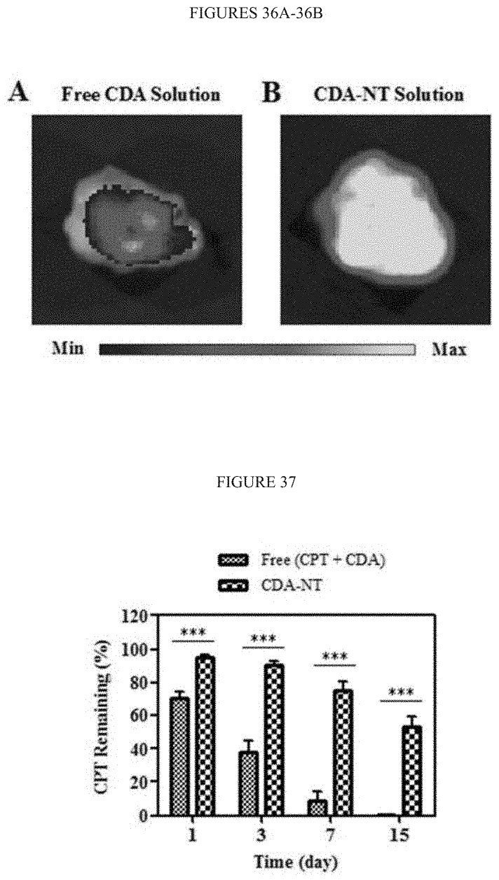

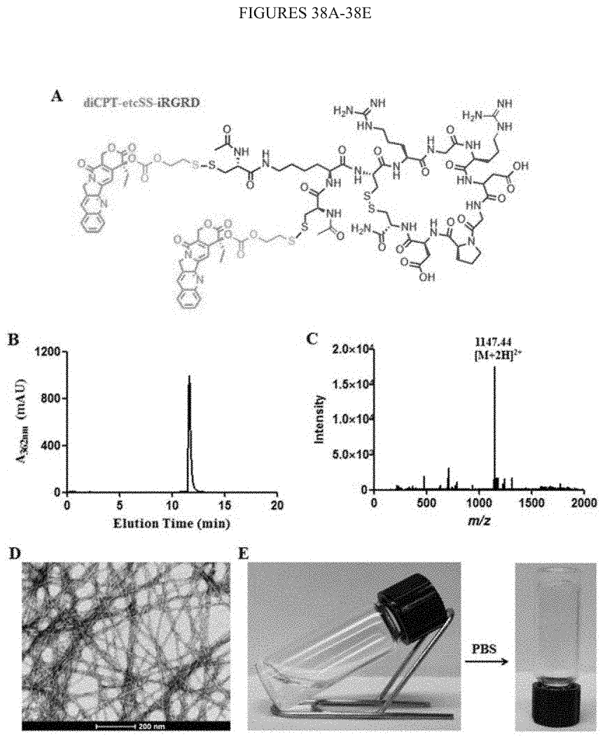

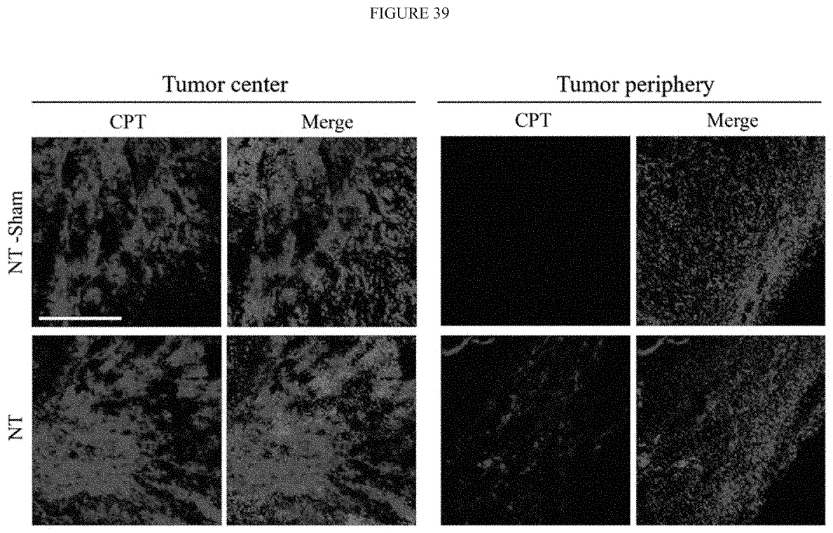

To further assess the potential of this CDA-NT hydrogel as a drug depot, the intratumoral therapeutic release behavior was investigated on GL-261 tumor-bearing mice with a Cy7-labeled CDA. The fluorescent CDA was first mixed with a NT aqueous solution, and the mixture was injected into the tumors, with free Cy7-labeled CDA solution (not loaded with NT) as control. The CDA signal was detected using fluorescence IVIS imaging (FIG. 2C and FIG. 35) and the data were quantified (FIG. 2D). As expected, the fluorescence intensity of free CDA treated tumors dropped rapidly, virtually no fluorescence signal observed five days after injection. In stark contrast, the fluorescence intensity for the CDA-NT treated group remained strong on day 15 and was still detectable even on day 35 after injection (FIG. 35). These results further confirmed that the NT hydrogel could significantly prolong the drug retention time in tumors and extend the local release of CDA. It is worth mentioning here that the specific deposition of STING agonist in the tumor tissue might also reduce the off-target toxicity and immune-related adverse events. Moreover, the IVIS and CLSM imaging of treated tumors after three days revealed that very low fluorescence was detected at the injection site for tumors treated with free CDA (FIG. 36). The CDA-NT treated group, on the other hand, displayed strong fluorescence throughout the entire tumor, confirming effective drug delivery by the diCPT-iRGD NT hydrogel. These results were further validated by confocal imaging of tumor tissue sections (FIG. 2E). Again, CDA signal was observed to drop significantly in the free CDA treated tumors, whereas CDA-NT treatment led to enhanced fluorescence in the tumor. Importantly, CPT was observed to release from CDA-NT hydrogel over an extended time period (FIG. 37). To determine whether iRGD can indeed facilitate tumor tissue penetration, a control drug amphiphile with a scrambled peptide sequence, diCPT-iRGRD, was designed and synthesized (FIG. 38). This drug amphiphile can also form self-supporting NT hydrogel (defined as NT-Sham) under the physiological condition. As depicted in FIG. 39, both NT-Sham and NT treated tumors exhibited very strong CPT fluorescence in the tumor center, suggesting great local retention. However, for mice treated with NT-Sham hydrogel, no blue fluorescence was observed at the periphery of tumor. This is in sharp contrast with the diCPT-iRGD treated mice, in which distinct CPT fluorescence can be observed throughout the tumor and even at the tumor periphery.

### Example 12

This example demonstrates that CDA-NT hydrogel elicits regression of established tumors through STING-dependent immune stimulation.

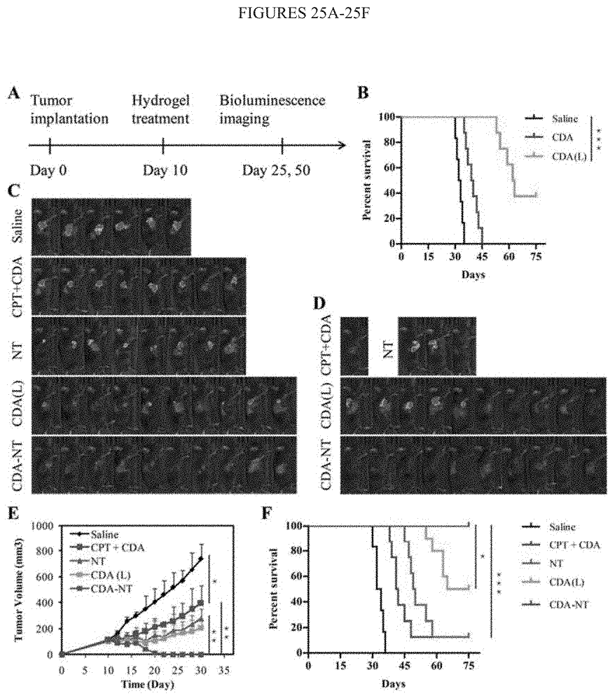

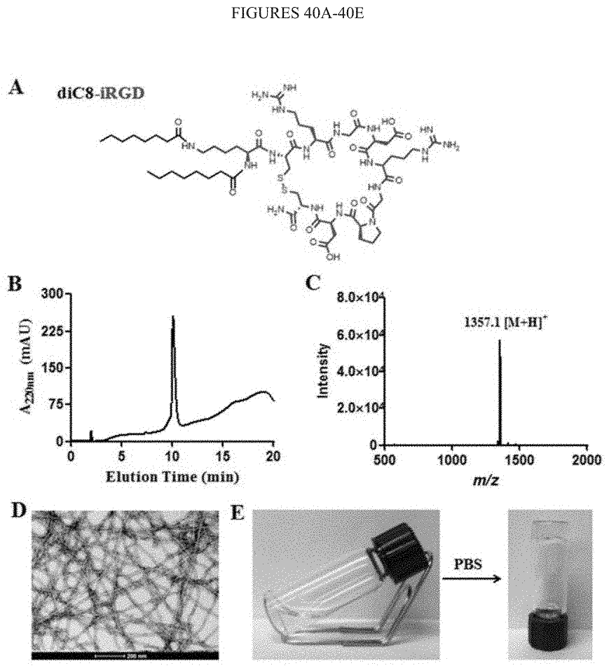

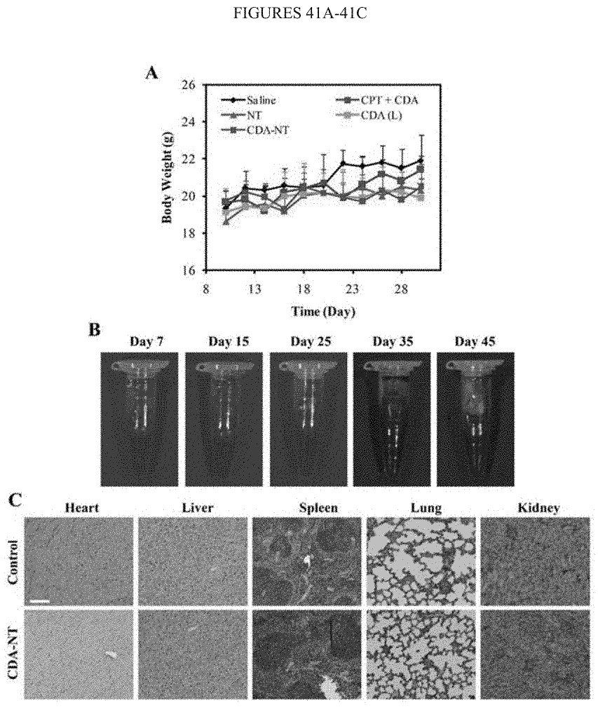

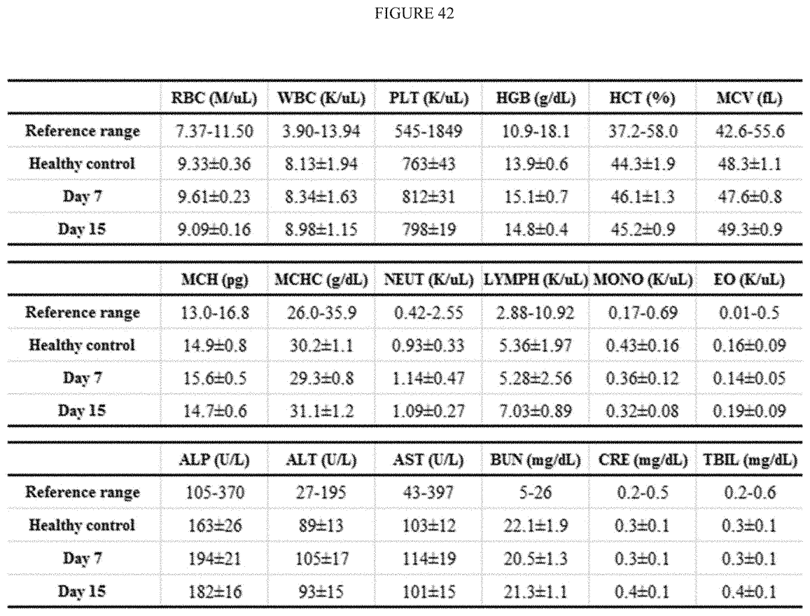

Having confirmed that the release of therapeutic agents could be sustained locally in vivo, it was decided to evaluate the synergistic antitumor effects of the CDA-NT hydrogel on subcutaneous GL-261 brain tumor model. The CDA-NT solutions were injected into the tumor when the tumor volume reached ˜100-150 mm³ at day 10 post-tumor inoculation (FIG. 25A). To better assess the synergy of combining chemotherapeutics with STING agonists, diC₈-iRGD was designed and used as a drug-empty hydrogel (E-Gel) (FIG. 40) for the local delivery of CDA [CDA(L)]. Given that the E-Gel also enhances local retention and release of the STING agonist in tumors, treatment with CDA(L) resulted in a significant increase in the survival rate relative to free CDA. But the monotherapy by CDA(L) alone was not sufficient to completely control the tumor burden (FIG. 25B). When the diCPT-iRGD NT hydrogel is used, the CDA-NT treatment led to remarkable tumor regression (FIGS. 25C, D and E) and demonstrated a 100% survival rate in mice (FIG. 25F). Chemotherapeutic NTs and immunotherapeutic CDA(L) alone resulted in 12.5% and 50% mice survival, respectively. When combined together as non-encapsulated free CPT+CDA, the treatment exhibited much less effective tumor growth inhibition than CDA-NT, suggesting the superior therapeutic properties of NT hydrogels over free CPT solution. Remarkably, the body weights of these tested mice were not impacted by local treatments (FIG. 41A). It should also be mentioned here that CDA was not detected in blood over 45 days post CDA-NT injection (FIG. 41B), and histology analysis of major organs, blood cell count and serum biochemistry showed no significant difference between CDA-NT treated mice and healthy mice (FIGS. 41C and 42), suggesting that local delivery of CDA-NT did not induce noticeable systemic adverse effects to the studied mice.

### Example 13

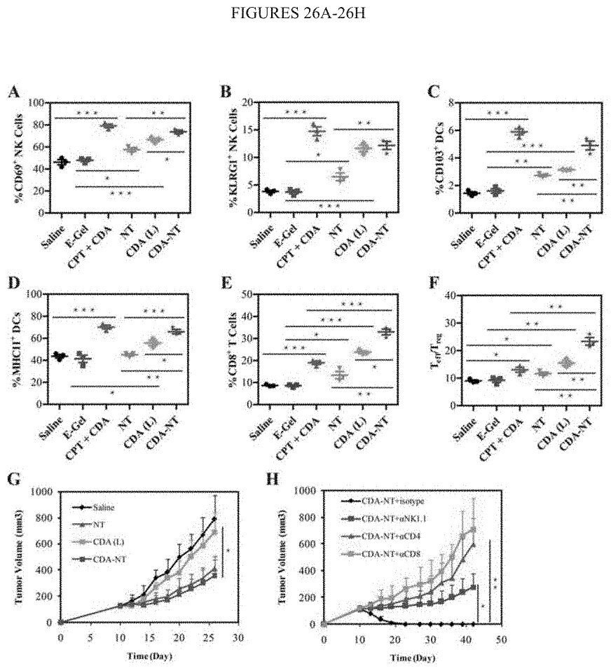

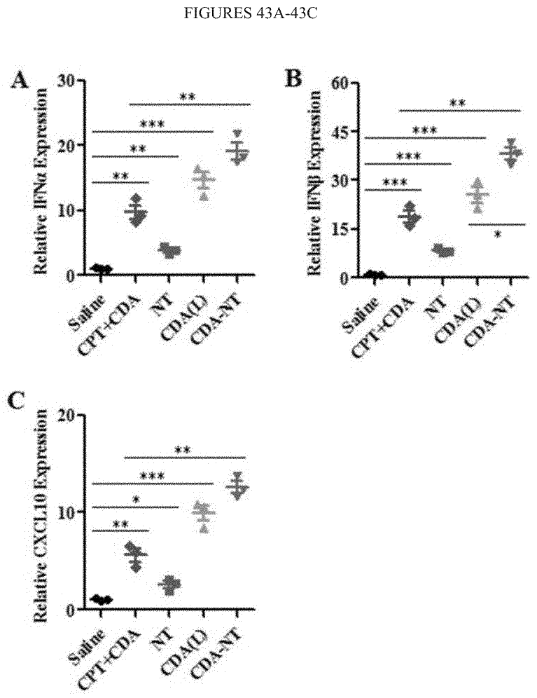

To dissect the changes in immune cell population within the TME after different treatments, the residual tumors were collected and analyzed using flow cytometry at day 3 and day 10 post-treatment. These results suggest that the diC₈-iRGD “drug-empty” hydrogel (E-Gel) had no effects on TILs. During the priming phase, the ratio of innate immune cells, particularly activated (CD69+ or KLRG1⁺) NK cells, was significantly increased after CPT+CDA, NT, CDA(L) and CDA-NT treatment (FIGS. 26A and B). Similar findings were observed for CD103+ and MHCII+ DCs (FIGS. 26C and D), both of which are essential for cross-presentation and DCs activation. Notably, CPT+CDA, NT, CDA(L) and CDA-NT markedly increased secretion of type I IFNs and CXCL10 chemokine (FIG. 43), which promote presentation of antigen to CD8+ T cells and recruitment of effector T cells to the tumor site. Indeed, these findings are further supported by the increased proportion of CD4+ T cells and CD8+ T cells within the tumor tissues (FIGS. 44A and B). Importantly, CDA-NT induced highest percentage of CD8⁺ T cells among all the treatment groups. At day 10 post-treatment, there was a decrease in the percentage of NK cells and DCs for CPT+CDA group (FIGS. 44 C-F), whereas mice treated with CDA(L) and CDA-NT maintained a high proportion of NK cells and DCs. Since DCs are known to stimulate adaptive immune system, the activity levels of CD4+ and CD8+ T cells were carefully examined. Indeed, the CDA-NT treated tumors exhibited higher infiltration of CD4+ and CD8+T (including IFN γ+ T cells) cells compared to other groups (FIGS. 26E, G and FIGS. 44 G-I). The percentage of CD8+ T cells in the CDA-NT treated mice was observed to increase 1.7-fold over the CPT+CDA group and 2.5-fold over the NT treated mice. Furthermore, the ratio of tumor-infiltrating CD8⁺ T cells (T_{eff}) to CD4+Foxp3⁺ regulatory T cells (Treg) was also observed to increase substantially after CDA-NT treatment (FIG. 26F). These observations suggest that local delivery of CDA-NT promotes an immune-stimulating TME with both innate and adaptive immune cells infiltrated into tumor tissues so as to induce a robust antitumor immune response.

### Example 14

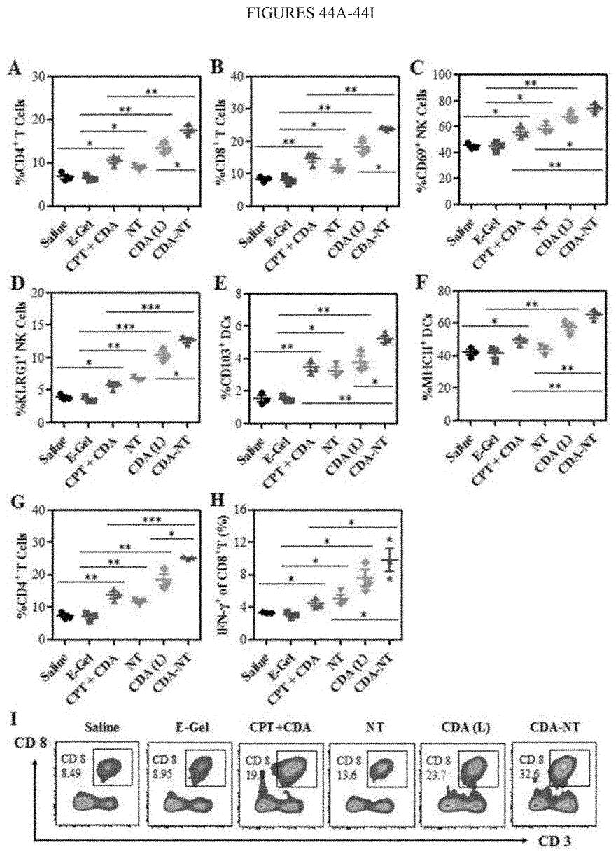

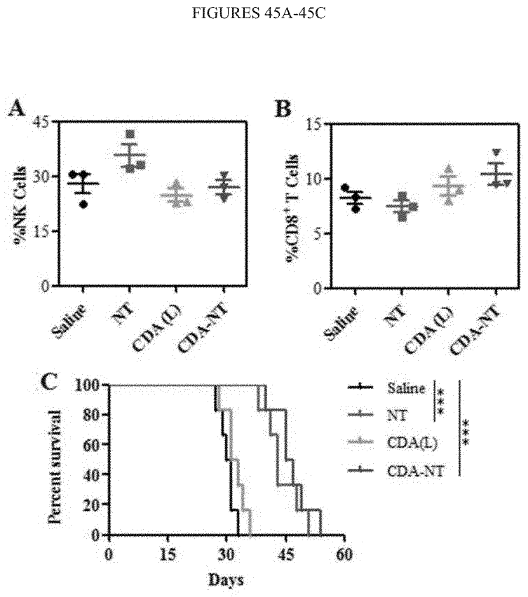

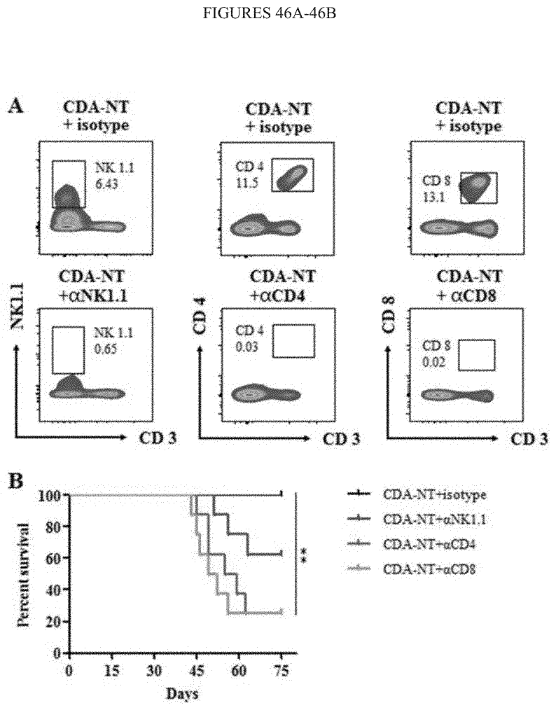

To determine whether the observed anti-tumor efficacy of CDA-NT was STING-dependent, experiments were performed on GL-261 tumor-bearing STING-deficient (STINGgt/gt) mice. Analysis of tumor tissues removed from STINGgt/gt mice revealed lack of NK cells and CD8+ T cells accumulation in tumors after each treatment with the NT, CDA(L) and CDA-NT (FIGS. 45A and B). Compared with the anti-tumor activity in WT (C57BL/6) mice (FIG. 25E), nearly no therapeutic efficacy was seen in CDA(L) treated STINGgt/gt mice (FIG. 26G, FIG. 45C). Importantly, the treatment efficacy by NT alone was also reduced in STINGgt/gt mice in comparison to WT mice. CDA-NT also exhibited modest tumor growth reduction in STINGgt/gt mice, and the observed anti-tumor efficacy of CDA-NT could be ascribed to the cytotoxic nature of CPT and CDA. These results provide strong evidence that STING pathway serves as a prerequisite for CDA induced tumor inhibition and is critical to maximize the therapeutic outcomes of CDA-NT treatment. To further investigate the contribution of the individual immune cell to the robust therapeutic efficacy of CDA-NT, NK cells, CD4+ T cells or CD8+ T cells was depleted by using appropriate antibodies (FIG. 46A). Depletion of NK cells partially impaired the anti-tumor effect of CDA-NT, whilst depletion of CD4+ T cells or CD8+ T cells resulted in a significant decrease in both tumor growth inhibition and survival benefit (FIG. 26H and FIG. 46B). These results confirmed that CDA-NT elicits tumor regression through priming NK cells, CD4+ T cells and CD8+ T cells, and that anti-tumor effect of CDA-NT is critically dependent on adaptive immune response. Collectively, these findings suggest that CDA-NT induces both innate and adaptive immune systems by activation of the STING pathway.

### Example 15

This example demonstrates that local delivery of CDA-NT induces durable immune responses.

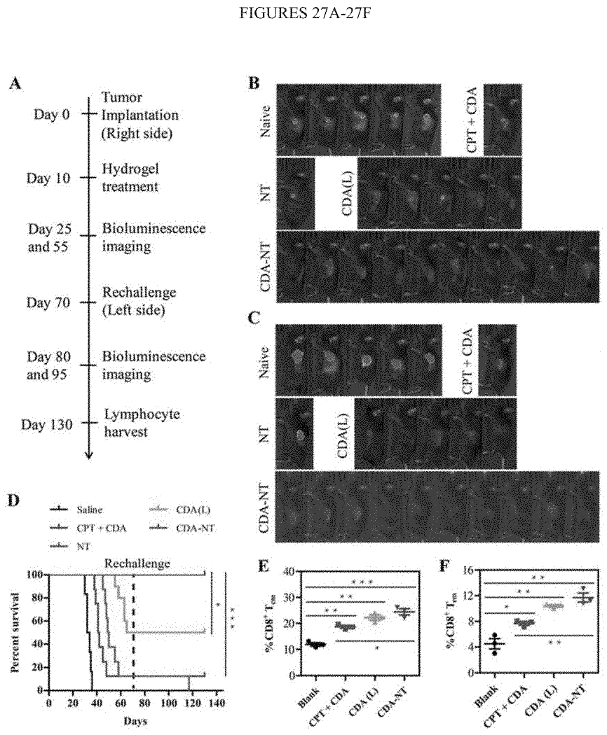

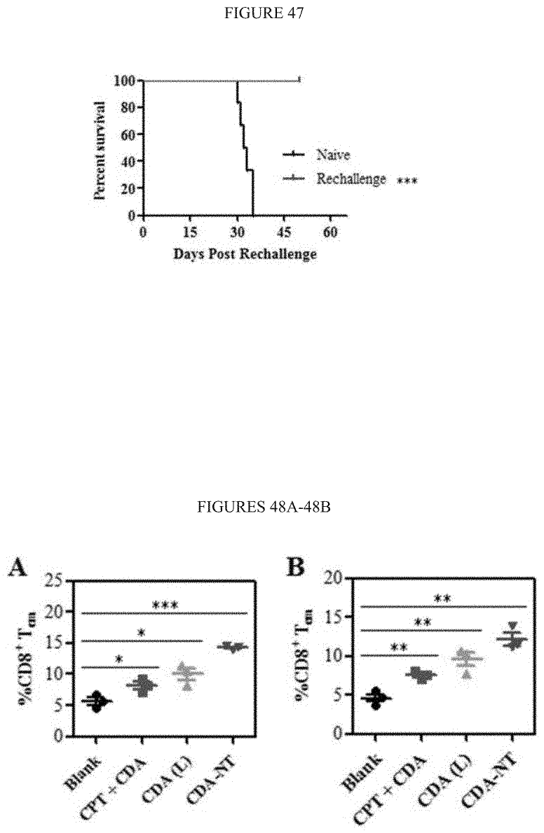

To examine whether the local delivery of the CDA-NT hydrogel also induces a memory response, mice with long-term survival from all groups were rechallenged on day 70 to see if new tumors can be developed on the opposite flank, and naive mice were challenged as control (FIG. 27A). As shown in FIGS. 27B and 27C, tumor development can be clearly seen in naive mice on day 10 after tumor cell inoculation and continued to grow into large tumors on day 15. The only survived mouse in the NT treatment group also developed tumors after rechallenge that grew over time. For mice survived in the CPT+CDA, CDA (L) and CDA-NT groups, although some initial tumor growth was seen in several mice on day 80 (10 days after inoculation of tumor cells), the bioluminescence signal of GL-261 cells was not detectable on day 95, suggesting effective inhibition of tumor growth in those survived mice. It should be noted that the overall survival of the CDA-NT treated mice was significantly higher than that of other treatment groups (FIG. 27D and FIG. 47). Sixty days after tumor rechallenge, spleens and peripheral blood were collected and analyzed using flow cytometry to examine memory immune cells. Indeed, the percentages of CD8+CD44highCD62Lhigh central memory T (Tcm) cells and CD8+CD44_{high}CD62_{low} effector memory T (Tem) cells were both elevated in mice treated with the STING agonist (FIGS. 27E and F, FIG. 48). CDA-NT treated mice exhibited higher percentages of CD8+ Tcm and Tem cells over the controls. These results indicate that local delivery of CDA-NT induces T cell memory and the establishment of long-term immunity against tumor recurrence.

### Example 16

This example demonstrates that the CDA-NT hydrogel promotes regression in other tumor models.

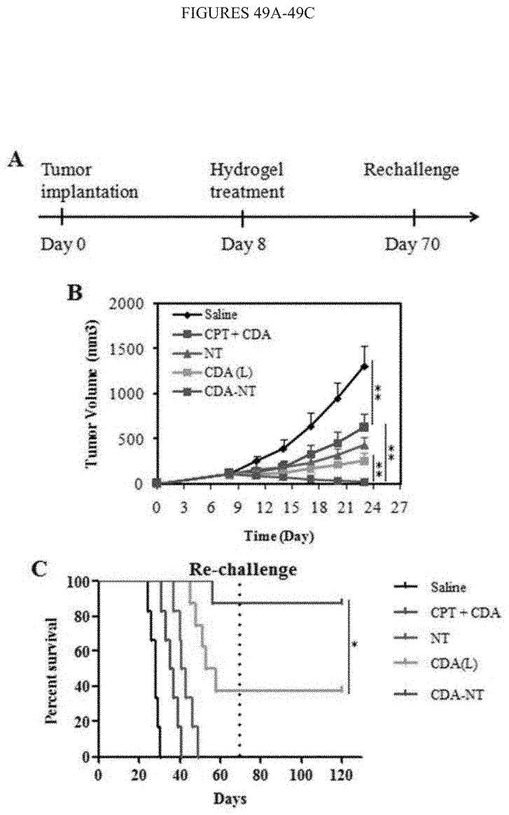

To further demonstrate the broad application of the proposed CDA-NT chemo-immunotherapy hydrogel, CT 26 colon cancer-bearing BALB/c mice were evaluated. When the tumor volume reached ˜100-150 mm³ at day 8 post-tumor inoculation, the in situ formed gels were injected into the mice (FIG. 49A). Consistent with amplified anti-tumor efficacy in the brain tumor model (FIG. 25), the combined chemo-immunotherapy of CDA-NT led to complete regression of established CT26 colon carcinoma tumors in ˜88% of mice (FIGS. 49 B and C).

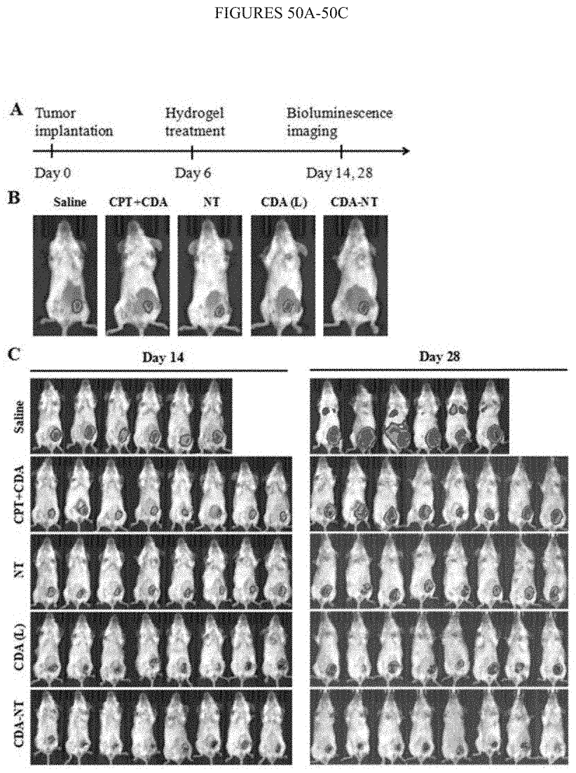

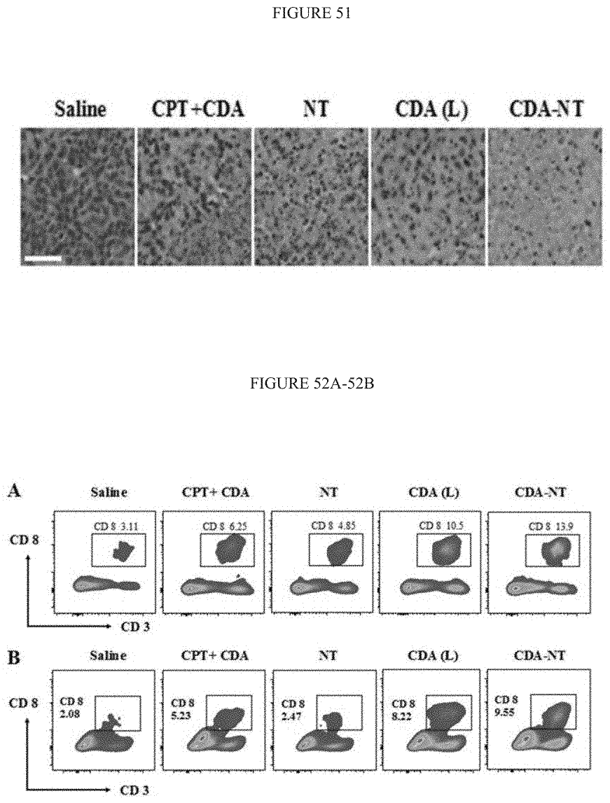

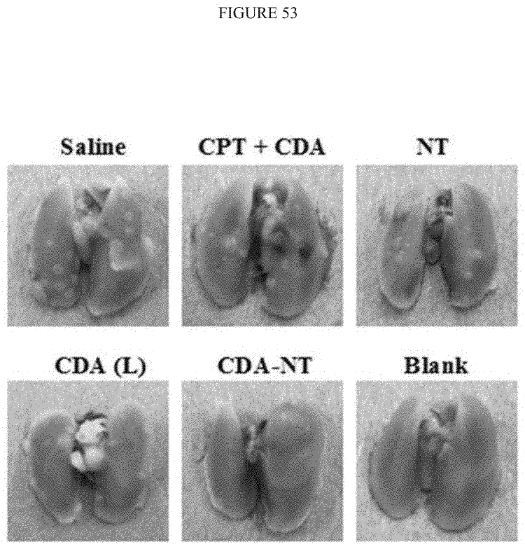

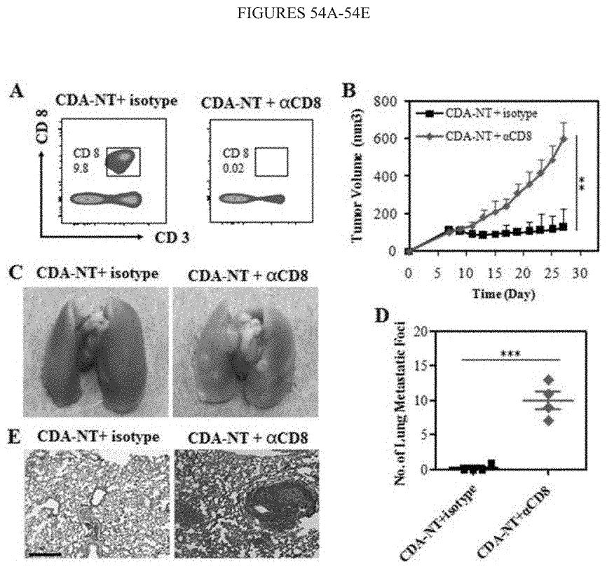

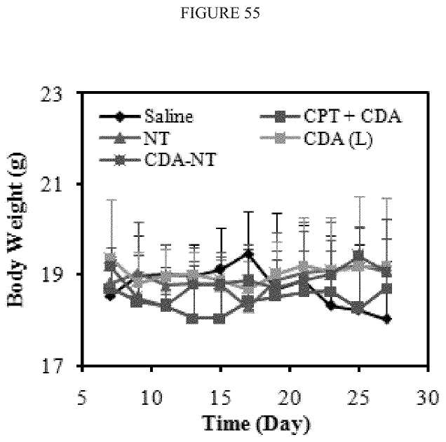

Furthermore, the efficacy of CDA-NT was evaluated in a low-immunogenic 4T1 breast cancer model, where 4T1 breast cancer cells were implanted into the mammary gland of female BALB/c mice. After 6 days, tumors were treated with different therapeutic agents (FIGS. 50 A and B). Tumor growth was significantly inhibited in the mice treated with CDA-NT (FIG. 50C and FIG. 28A), which led to complete tumor regression in 50% mice (FIG. 28B). These results are mainly ascribed to the CPT-induced cell death (FIG. 51) and the STING-agonist-promoted robust antitumor immune response, given that increased infiltration of activated NK cells, DCs, and CD8+ T cells were detected in the CDA-NT treated tumors (FIG. 28C-E and FIG. 52A). It should be noted that extensive tumor nodules were found in the lung tissue of the Saline group mice (FIGS. 50C and 53), indicating that the 4T1 breast cancer underwent significant pulmonary metastasis. CPT+CDA, NT and CDA (L) therapy substantially decreased, but not completely inhibited, lung metastasis, as confirmed by H&E staining (FIG. 28F) and whole lung observation (FIG. 53 and FIG. 28G). Remarkably, the CDA-NT hydrogel possessed the greatest anti-metastatic efficacy, with no metastatic nodules observed in the lung. This could be explained by the systemic immune response induced by CDA-NT, corresponding to increased CD8+ T cells in the peripheral blood (FIG. 28H and FIG. 52B). The contribution of the immune system to the observed efficacy was further assessed using a CD8⁺ T cell depletion assay. Indeed, depletion of CD8+ T cells significantly reduced the treatment efficacy of CDA-NT, with impaired tumor growth inhibition and increased lung metastasis tumor nodules (FIG. 54), demonstrating that CDA-NT induced CD8+ T cells were critical to inhibit 4T1 tumor growth and metastasis. Furthermore, splenocytes harvested from treated mice were re-stimulated with AH1 peptide, an immunodominant gp70 (amino acids 423-431) endogenous retroviral antigen that is expresses by 4T1 cells. The proportion of CD8+ T cells secreting IFNγ were significantly increased in mice treated with CDA-NT hydrogel (FIG. 28I). These results provided compelling evidence that the CD8+ T cell responses to AH1 peptide re-stimulation in a tumor antigen-specific manner, and that the observed anti-cancer effect of CDA-NT hydrogel is associated with tumor antigen-specific CD8+ T cell response. Additionally, the body weights of mice in the CDA-NT group showed no significant difference after treatment, indicating that this in situ-formed hydrogel does not cause severe systemic toxicity (FIG. 55).

### Examples 17-22

Glioblastoma multiforme (GBM) is the most aggressive neoplasm, with extremely high patient morbidity and mortality. Current standards of care for GBM focus on maximal safe surgical resection, implantation of GLIADEL® wafers (the only FDA approved carmustine implant) in the resection site adjuvant radiotherapy and/or oral chemotherapy. Despite an initial modest therapeutic effect, infiltrating cancerous cells remain in the surrounding brain parenchyma following resection, leading to tumor recurrence and thereby limiting the 5-year survival rate to below 5%. While recent progress in immunotherapy has drastically improved clinical outcomes for patients with a few advanced cancers, brain tumors continue to be a conspicuous exception to this trend, largely due to the unique immunosuppressive tumor environment in brain and insufficient infiltration of T cells into GBM. Even the most prominent immunotherapy approaches such as checkpoint inhibition, PD-1 and CTLA-4 blockade, and CAR-T cell therapies have shown limited benefit in GBM patients to date. Given that tumor-associated microglia/macrophages (TAMs) constitute 30-50% of brain tumor mass, macrophage-directed immunotherapy may improve the treatment of GBM.

CD47 blockade has demonstrated potential in clinical trials 23, but it requires multiple large doses and combination with chemotherapy or irradiation for an efficacious treatment of malignant brain tumors 13, 14, 22, 24. Moreover, systemic administration of CD47 antagonists causes severe side effects such as anemia and thrombocytopenia and its efficacy is compromised due to aCD47 sequestration by red blood cells 25-27. As such, approaches to enhance aCD47 immune activation and to avoid off-target effects are needed for CD47-blockade-based immunotherapy in GBM patients.

Paclitaxel (PTX) is the most successful drug in cancer chemotherapy and has been approved for the use against a wide variety of cancers including GBM. Recently, there has been mounting evidence to support that PTX can trigger infiltration of TAMs and induce enrichment of CD47 on cancer cells. These findings suggest that PTX has the ability to boost the immune response in a manner synergistic with CD47 blockade immunotherapy. It has been shown that direct linkage of a chemotherapeutic agent onto a biologically active β-sheet forming peptide transforms the drug into a supramolecular hydrogelator. The rapid solution-to-hydrogel phase transition of such self-assembling prodrug systems under physiological conditions allows for their deposition and retention in the resection cavity immediately after surgical removal of GBM. Based on these observations, it was reasoned that such PTX-containing in situ formed hydrogels can be exploited for localized delivery of immunotherapeutic agents, enabling a sustained, directed release of the combination therapy into the surrounding brain tissues to eradicate residual tumor cells.

In this context, a PTX-bearing supramolecular hydrogel was developed for site-specific delivery of aCD47 and PTX. The experiments described below reveal that the aCD47-loaded PTX prodrug hydrogelator forms a well-defined hydrogel upon infusion into the resection cavity. The hydrogel serves as a drug depot for localized, sustained release of both PTX and aCD47, eliciting an immune-stimulating tumor microenvironment (TME) and inducing macrophages phagocytosis of cancer cells.

The following methods and materials were employed in Examples 17-22.

### Materials, Cell Lines and Animals

All amino acids and Rink Amide MBHA Resin were purchased from AAPPTEC (Louisville, Ky.). Anti-CD47 antibody (cat. no. 127519) was obtained from BioLegend. All other reagents and solvents were purchased from Sigma-Aldrich. GL-261-luc cells were obtained from M. Lim at The Johns Hopkins University. Cells were cultured in DMEM (Gibco, Invitrogen) supplemented with 100 μg/ml of G418 (Invitrogen). 8-10-week female C57BL/6 mice were purchased from Charles River. Animal experiments were carried out following the animal protocol approved by the Animal Care and Welfare Committee at The Johns Hopkins University.

### Synthesis of PTX-iRGD

Through the standard Fmoc-solid phase technique, an AAPPTEC Focus XC synthesizer was used to synthesize the peptide C₂K-cyl[CRGDRGPDC]. RP-HPLC and MALDI-TOF MS were used to purify and analyze the peptide, respectively. Paclitaxel (PTX) was chemically modified to PTX-buss-Pyr as previously reported. Then, PTX-buss-Pyr and purified peptide were mixed at a molar ratio of 1:1.5 (PTX-buss-Pyr/peptide) in 5 ml N²-purged DMSO and allowed to react for 3 days. The crude reaction solution was purified by RP-HPLC, then the product was confirmed by ESI MS and lyophilized to obtain PTX-iRGD as a white powder.

Preparation of aCD47/PF Hydrogel

PF hydrogel formation was tested by adding 15 μl of 10×PBS to 150 μl of 17.5 mM PF solution. For preparation of aCD47 loaded PF hydrogel (aCD47/PF), aCD47 (50 μg) was added to the PF solution (200 μg of PTX). After vortexing, 10×PBS was added to the mixture to induce the formation of aCD47/PF hydrogel. The inverted-vial method was then performed to test sol-to-gel transition. aCD47 dispersion in the hydrogel was further characterized using confocal microscopy (Zeiss LSM 510). For in vivo application, aCD47 and PF mixture solution was loaded into a syringe and directly injected into the target sites to form a gel in situ.

### Hydrogel Degradation Study

100 μl of 17.5 mM PF solution was subcutaneously injected into C57BL/6 mice. At predetermined time points, the mice were sacrificed and the remaining hydrogel in each mouse was photographed. Hydrogel weights were determined by detecting the amount of PTX-iRGD within the hydrogel.

Drug Release from aCD47/PF Hydrogel

The in vitro release study was performed at 37° C. in PBS with 10% FBS. The released PTX-iRGD was quantified by HPLC, whereas released aCD47 (labeled with Cy3) was analyzed using a fluorescence spectrophotometer. To evaluate the in vivo release of aCD47, free aCD47 or aCD47/PF solutions were injected into the subcutaneous GL-261-luc tumors, which were generated by inoculating 2.5×10⁶ cells on the right flanks of female C57BL/6 mice for ten days. At predetermined time points, fluorescence imaging of Cy5.5-aCD47 was completed using an IVIS Spectrum imaging system (Perkin Elmer). Harvested tumors were also cut into sections, stained with DAPI, and imaged using a confocal microscope (Zeiss LSM 510). Furthermore, to evaluate the release of aCD47 in the brain, the mice were intracranially implanted with aCD47/PF hydrogel. Briefly, mice were anesthetized and stereotactically injected with 10 μl of aCD47/PF solution using a 25 μl Hamilton syringe fitted with a 26 G needle. The injection coordinates were 2.5 mm lateral, 0.5 mm anterior to bregma, and 2.5 mm deep from the outer border of the cranium. At predetermined time points, brains were taken out and fluorescence imaging of Cy5.5-aCD47 was completed using an IVIS Spectrum imaging system (Perkin Elmer).

### Glioblastoma Tumor Model and Local Treatment

An orthotopic glioblastoma tumor model was developed by intracranial implant of GL-261 glioma cells. Mice were anesthetized by intraperitoneal injection of ketamine/xylazine. Once prepped for surgery, the mice were positioned on a stereotactic frame and a 5 mm long midline scalp incision was made. A burr hole was drilled at the right cranial hemisphere, 2.5 mm lateral and 0.5 mm anterior to bregma. Then, 1×10⁵ GL-261-luc cells were implanted at a depth of 2.5 mm from the dura. The skin was closed with tissue glue. On day 6 after inoculation, brain tumor-bearing mice were randomly assigned to five groups (n=8). The cranium was re-opened and 10 μL of treatment solutions were injected in the previous burr hole using a 25 μL Hamilton syringe fitted with a 26 G needle. The treatment groups were as follows: Saline, drug free DOCA-iRGD filament solution (EF), PTX-iRGD filament solution (PF), aCD47 loaded DOCA-iRGD filament solution (aCD47/EF), or aCD47 loaded PTX-iRGD filament solution (aCD47/PF). Administered drug doses were 50 μg of aCD47 and 150 μg of PTX per mouse. Body weights and behavior of the mice were monitored daily post-operation and treatment. Mice were euthanized when body weight loss surpassed 20%.

### Orthotopic GBM Tumor Resection Model and Treatment

At day 8 post-tumor implantation, well-established tumors had formed and the tumor resection was performed using method similar to those previously reported 44, 55. Briefly, mice were anesthetized, then immobilized on a stereotactic frame and a midline incision was made in the skin above the cranium to expose the previous burr hole. Under a dissecting surgical microscope, the brain tumor was surgically resected using a biopsy punch. Tumor tissue and blood were aspirated using a vacuum pump. Then, 10 μL of formulation solution was injected into the resection cavity using a 25 μL Hamilton syringe fitted with a 26 G needle. Finally, the wound was closed with tissue glue. The treatment groups were as follows (n=8 for each group): Saline, EF, PF, aCD47/EF, or aCD47/PF. The dosages of administered drugs were 50 μg of aCD47 and 150 μg of PTX per mouse.

Mice were considered long-term survivors if no tumor was detected 80 days post-tumor implantation. Long-term survivors from all treatment groups were rechallenged at day 80 with 2×10⁵ GL-261-luc cells in the contralateral hemisphere to develop new tumors. Naïve mice were intracranially implanted as controls. Tumor growth was checked weekly using IVIS imaging. Body weight and behavior of the mice were also monitored post-surgical resection and treatment. Mice were euthanized when body weight loss surpassed 20%.

### Brain Tumor Growth Monitoring

Tumor growth in the brain was monitored by bioluminescence imaging using an IVIS Spectrum Imaging System. Bioluminescence images were analyzed using Living Image software (Perkin Elmer). The location and size of brain tumors were also tracked by magnetic resonance imaging (MRI) using a horizontal bore 4.7 T Biospec animal imager (Bruker Biospin). T2-weighted images were acquired in the horizontal plane at predetermined time points. Additionally, to examine the pattern of tumor growth, mice were sacrificed at different days and specimens of the brain were harvested, fixed and stained with haematoxylin and eosin (H&E). The H&E brain slides were then imaged using an optical microscope.

### Safety Study of Hydrogel in Healthy Mice

10-week-old healthy female C57BL/6 mice were anesthetized, a burr hole was made as described above, then 10 μL of aCD47/EF solution was injected in the burr hole using a 25 μL Hamilton syringe fitted with a 26 G needle. At predetermined time points, blood samples were harvested and complete blood cell count and serum biochemistry were determined. After 1 month, mice were sacrificed, the brain and other major organs were harvested, fixed, and stained with H&E. The slides were then imaged using an optical microscope.

### Flow Cytometry

Tumors collected from mice were digested with 1 mg/mL Collagenase digestion buffer (Type IV, Gibco) to get single cell suspensions. Cells were then stained with fluorescence-labelled antibodies following the manufacturer's instructions. Flow cytometry was performed using a FACSCanto II instrument (BD Biosciences) and analyzed by FlowJo software (Tree Star). Antibodies against CD45 (30-F11), CD11b (ICRF44), F4/80 (BM8), CD3 (17A2), CD4 (RM4-5), CD8 (53-6.7), Foxp3 (FJK-16s), Gr-1 (RB6-8C5), CD80 (16-10A1), CD86 (GL-1), CD103 (2E7), CD44 (IM7), and CD62L (MEL-14) were purchased from Biolegend or eBioscience. To detect the expression of CD47 on the tumor cells, cells were stained with FITC-labelled anti-CD47 antibody (miap301) and measured using flow cytometer.

### Cytokine Analysis

Blood samples were collected from mice on day 7 post-treatment. Serum levels of IL-6, IL-12, IFN-γ, and TNF-α were measured using ELISA kits according to the manufacturer's instructions.

### Statistical Analysis

All results are presented as means±SD. The two-tailed unpaired t-test was used to determine statistical significance between two treatment groups and ANOVA was used for multiple comparisons. Survival was plotted using a Kaplan-Meier curve and assessed by a log-rank (Mantel-Cox) test. Statistical analysis was performed using GraphPad Prism software 5. *P≤0.05, **P≤0.01, ***P≤0.001.

### Example 17

This example describes the characterization of aCD47/PF supramolecular filament hydrogel.

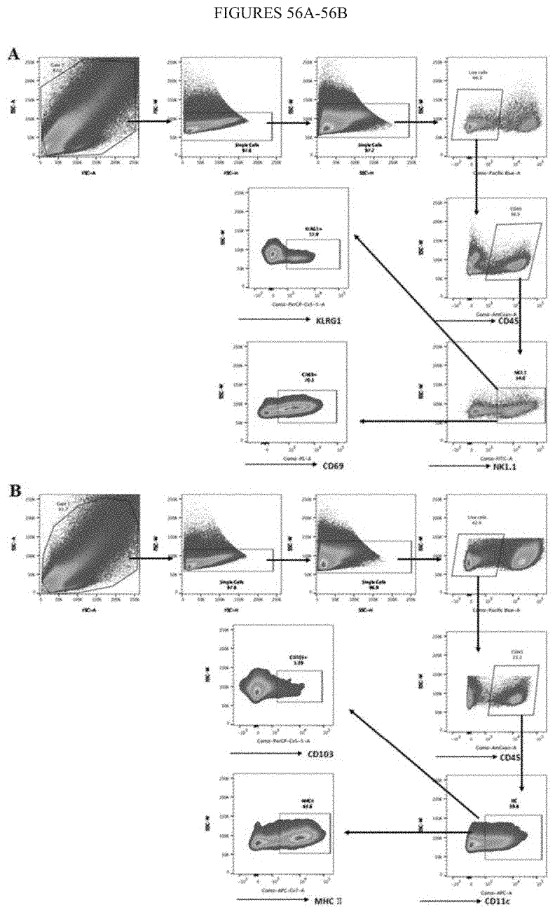

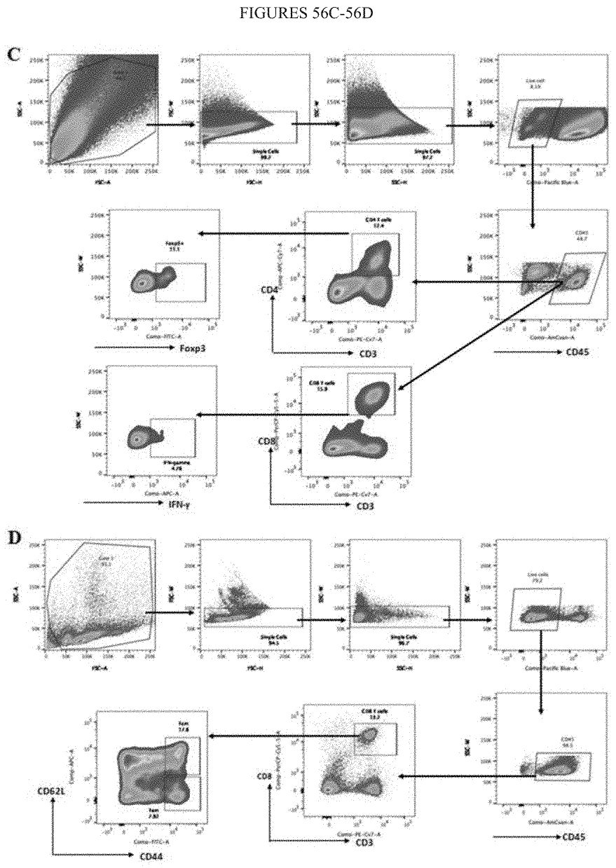

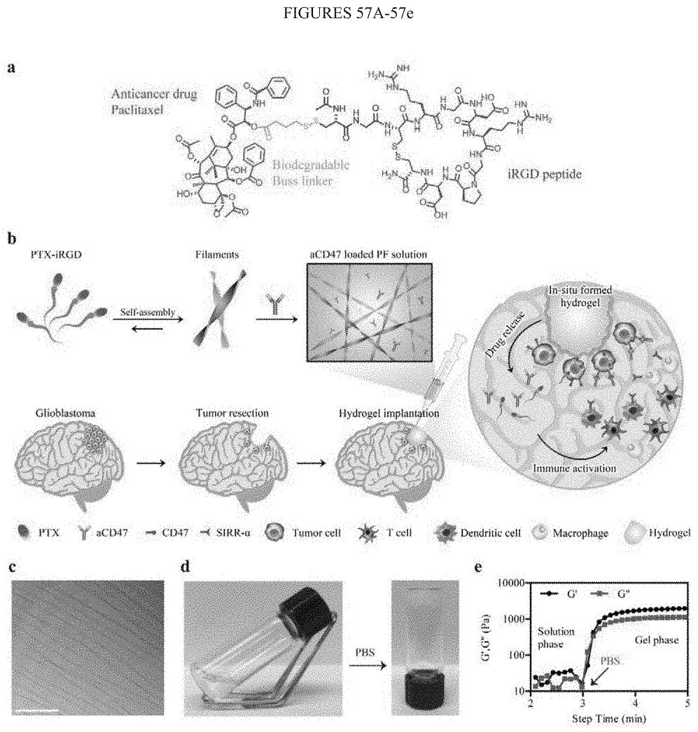

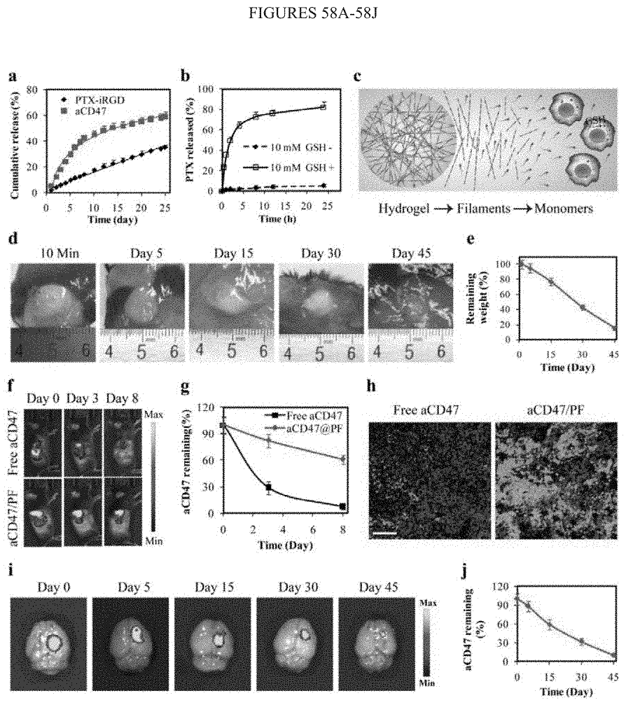

To construct a bio-responsive hydrogel, a PTX prodrug hydrogelator (PTX-iRGD) was synthesized by chemically conjugating a PTX molecule to the iRGD peptide through a reducible 4-(pyridin-2-yl-disulfanyl)butyrate (buSS) linker using a previously reported method 50 (FIG. 57A). iRGD was incorporated into the molecular design since it enhances penetration of tumor tissues by binding to neuropilin-1, leading to transcytosis. This rationally designed amphiphilic PTX prodrug spontaneously assembled into supramolecular filaments upon dissolution in water (FIG. 57C). The addition of PBS or cell medium (FIG. 57D) to PTX filament (PF) aqueous solution induced rapid hydrogel formation due to charge screening between filaments. The solution-to-hydrogel transition of PF was confirmed using rheological testing. Addition of PBS to the PF solution drastically increased the storage modulus (G′), indicating the formation of a supramolecular hydrogel (FIG. 57E). It was hypothesized that the rapid solution-to-hydrogel transition of this PF hydrogelator may make it suitable to provide for localized controlled delivery of aCD47 monoclonal antibodies (mAbs). By simply mixing aqueous solutions of aCD47 and PF, followed by gelation induction by PBS, aCD47 was loaded into the hydrogel (aCD47/PF). Moreover, confocal imaging revealed spatially uniform distribution of aCD47 throughout the hydrogel (Supplementary FIG. 58B).

The release of PTX-iRGD and aCD47 from aCD47/PF hydrogel was then investigated in PBS at 37° C. PTX-iRGD exhibited a linear release profile from aCD47/PF hydrogel, with ˜35% PTX-iRGD released within 25 days. Approximately 30% of the encapsulated aCD47 was released over the first 5 days, followed by a gradually reduced release rate, with ˜60% of aCD47 released over 25 days (FIG. 58A). These results display a typical release profile for encapsulated antibodies from a hydrogel matrix and unique zero-order release for PTX-iRGD, suggesting that the hydrogel serves as a depot for sustained release of therapeutic agents, and for controlled release of chemotherapeutic hydrogelator. The intracellularly-rich reducing agent, glutathione (GSH), effectively liberates the parent PTX through the reduction of the GSH-responsive buSS disulfide linker utilized in the PTX-iRGD prodrug design. In the presence of GSH, the liberation of free PTX from PTX-iRGD by disulfide reduction took place very quickly, with ˜65% of parent PTX converted within 4 hours (FIG. 58B), implying PTX-iRGD could rapidly provide free PTX within cells (FIG. 58C). The PTX prodrug and its corresponding PF hydrogel achieved effective growth inhibition toward GL-261 tumor cells and tumor spheroids in vitro.

### Example 18

This example demonstrates that in situ formed hydrogel enhanced local retention of aCD47 in vivo and reduced side effects.

In vivo gelation was evaluated by subcutaneous injection of PF solution into mice. Injected PF transformed from a solution to a well-defined hydrogel within ten minutes (FIG. 58D). This in situ formed PF hydrogel exhibited gradual and nearly linear biodegradation by mass, with ˜82% hydrogel weight loss within 45 days (FIGS. 58D and 58E). These results suggest PF can serve as an in situ therapeutic depot at the target site for long-term release of therapeutics. Furthermore, to evaluate the in vivo retention behavior, Cy5.5-labelled aCD47 either in solution or encapsulated within PF solution were intratumorally injected into subcutaneous GL-261 tumor-bearing mice. Fluorescence signals in free aCD47 treated mice were detected in the tumor and major organs during the first three days, but the intensity dropped rapidly, with only ˜5% left at the tumor sites after eight days (FIGS. 58F and 58G). In sharp contrast, in the aCD47/PF injected mice almost all aCD47 was trapped within the tumors, with ˜62% of aCD47 remaining throughout a large tumor area after eight days (FIG. 58G). Confocal imaging of tumor sections further confirmed the PF hydrogel-induced local retention of aCD47, evident by strong fluorescence detected in aCD47/PF-treated tumors at three days following treatment (FIG. 58H,). These results suggest that in situ formed aCD47/PF hydrogel can significantly enhance drug retention within the target sites, and thus enable controlled release of aCD47 over a prolonged period of time.

Since the PF hydrogel can concentrate aCD47 at the desired site, it was hypothesized that localized aCD47/PF would reduce off-target toxicity and immune-related adverse events caused by conventional systemic administration of aCD47. To test this hypothesis, healthy mice were intracranially injected with aCD47/PF solution. Ex vivo imaging demonstrated that aCD47 retained in the injection site in the brain and the in situ formed hydrogel significantly prevented leakage of the aCD47 cargo (FIG. 58I). Although aCD47 was gradually released from the aCD47/PF hydrogel (FIG. 58J), fluorescent signals were hardly detectable in other major organs and blood over 45 days of investigation. Notably, body weight was not affected by intracranial injection of aCD47/PF. Moreover, histological analysis of major organs, blood test, and serum biochemistry assay showed no notable difference between aCD47/PF treated mice and healthy mice, suggesting that localized aCD47/PF did not cause obvious side effects.

### Example 19

This example demonstrates that aCD47/PF elicits regression of orthotopic GBM.

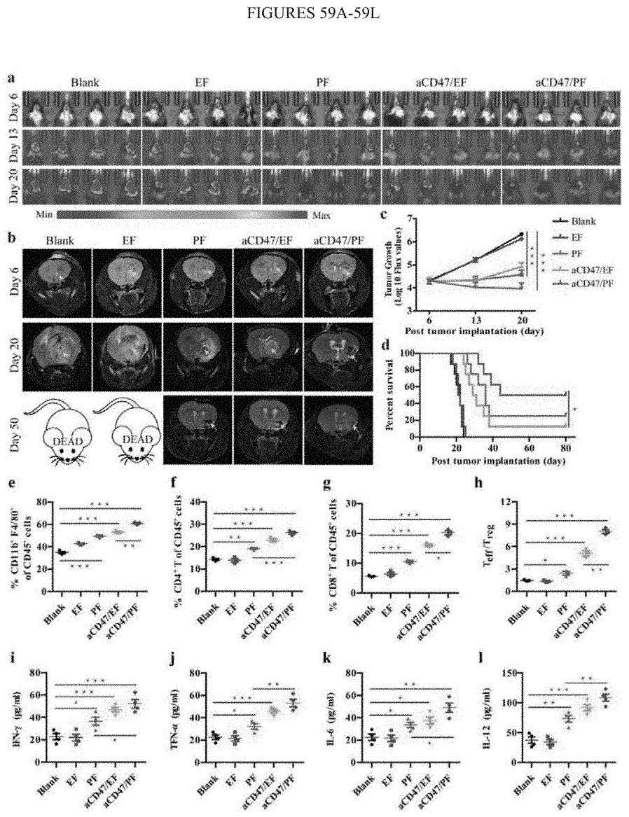

To evaluate therapeutic efficacy and investigate potential antitumor immune responses elicited by aCD47/PF hydrogel, an orthotopic GL-261 brain tumor model in mice was first utilized. For comparison, DOCA-iRGD was designed as a PTX-empty hydrogel (defined as EF gel) for the local delivery of aCD47 (aCD47/EF). The hydrogelators EF, PF, aCD47/EF, and aCD47/PF were directly injected into established brain tumors in mice. Tumor burden was monitored using bioluminescence signals and T2 weighted magnetic resonance imaging (MRI) (FIGS. 59A and 59B). As shown in FIGS. 59A-C, mice treated with “empty” EF hydrogel displayed a rapid and aggressive increase in tumor mass, and demonstrated no difference in survival compared to the untreated group (median survival, 21.5 vs 22 days) (FIG. 59D). In contrast, PF- and aCD47/EF-treated mice exhibited relatively slow tumor growth (FIGS. 59A and 59C) and the tumor lesion was substantially reduced by day 50 (FIG. 59B). 25% (median survival, 36 days) and 12.5% (median survival, 30 days) of the PF-treated and aCD47/EF-treated mice survived until the end of the study, respectively (FIG. 59D). Notably, aCD47/PF treatment dramatically inhibited brain tumor growth (FIGS. 59A-59C), with no tumor burden observed in long-term survival mice from MRI on day 50 (FIG. 59B). This combined treatment resulted in an extraordinary benefit in median survival (62 days), with 50% of aCD47/PF treated mice surviving until the end of the study (FIG. 59D).

Furthermore, residual tumors were collected, and the related immune cells were analyzed on day 10 post-treatment. All the therapeutic-containing hydrogels induced marked infiltration of CD45⁺ cells in tumor tissues. The percentage of macrophages was significantly increased after PF treatment and aCD47 blockade (FIG. 59E). This tendency was further enhanced by the combined aCD47/PF treatment (60.9% in aCD47/PF group vs. 49.6% and 53.1% in PF and aCD47/EF groups, respectively) (FIG. 59E). Furthermore, it was also observed that CD47 blockade increased proportion of antigen presenting CD103+DCs, and CD86+DCs, in consistency with previous studies on T cell-mediated antitumor immune response by aCD47-based therapy. Subsequently, both CD4+ (22.8% vs. 13.5% in Blank group) and CD8⁺ T (16.1% vs. 5.3% in Blank group) cell counts were significantly increased following aCD47/EF treatment (FIGS. 59F-59G). Among all treatment groups, aCD47/PF induced the highest percentages of CD4+ and CD8+ T cells. The proportion of CD8+ T cells in the aCD47/PF treated mice was 1.9-fold (P≤0.001) and 1.3-fold (P≤0.05) higher than those in the PF and aCD47/EF treated mice, respectively. The ratio of effector T cells (T_{eff}) to regulatory T cells (T_{reg}) was also significantly increased following aCD47/PF treatment (8.1% vs. 1.4% in Blank group) (FIG. 59H). The increased secretion of IFN-γ, TNF-α, IL-6, and IL-12 further validated the effective immune response elicited by aCD47/PF treatment (FIGS. 59I-59L). Together, these results suggest that aCD47/PF promotes a robust innate and adaptive antitumor immune response that dramatically inhibited brain tumor growth.

### Example 20

This example demonstrates that PF hydrogel elicits an immune-stimulating tumor microenvironment.

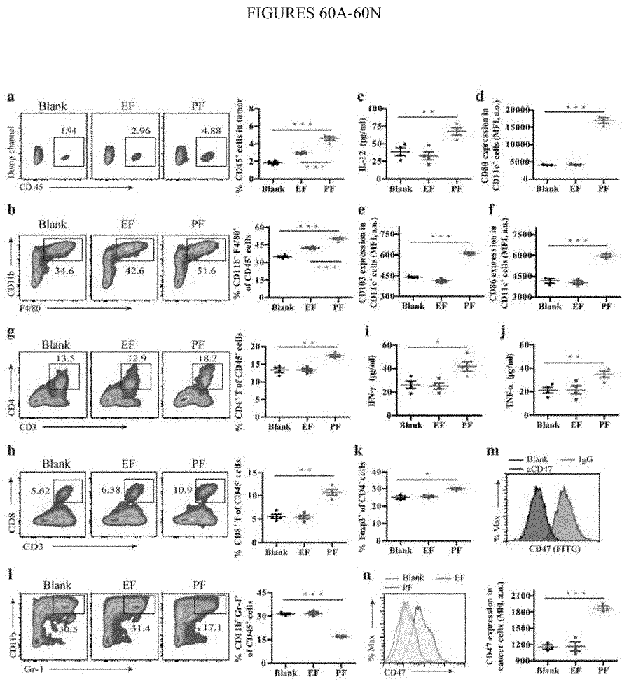

To test whether PF has the potential to boost the local immune response in a manner synergistic with CD47 blockade immunotherapy, the related immune cells and tumor cells were investigated after treatment of PF hydrogel. As shown in FIG. 60, the EF hydrogel had no obvious effects on tumor-infiltrating lymphocytes (TILs) and tumor cells. In contrast, localized PF hydrogel treatment significantly increased the percentage of CD45⁺ cells (4.6% vs. 1.8% in Blank group) and macrophages (50.1% vs. 34.8% in Blank group) in brain tumors (FIGS. 60A-60B). This result was supported by findings of an elevated interleukin-12 (IL-12) level in the TME which may be secreted from macrophages. PF treatment also increased the proportion of immune effector CD80+ dendritic cells (DCs), CD103+DCs, and CD86+ DCs within the tumor (FIGS. 60D-60F). These antigen-presenting DCs are known to stimulate adaptive immune responses. PF treated tumors exhibited increased percentages of CD4+ T (17.5% vs. 13.3% in Blank group), and CD8⁺ T (10.6% vs. 5.6% in Blank group) cells compared to control groups (FIGS. 60G-60H). This is consistent with previous reports that PTX can elicit DCs-mediated phagocytosis and subsequent activation of cytotoxic T lymphocytes (CTLs). The increased secretion of interferon-γ (IFN-γ) and tumor necrosis factor-α (TNF-α) further suggest an effective immune response elicited by PF treatment (FIGS. 60I and 60J). Although Foxp3+CD4+ T cells were slightly increased (FIG. 60K), a significant reduction of myeloid-derived suppressor cells (MDSC, CD11b+Gr-1+) (17.1% vs. 31.4% in Blank group) was observed in brain tumors treated with PF (FIG. 60L). As measured by flow cytometry, immune checkpoint CD47 is highly expressed on the surface of GL-261 brain cancer cells (FIG. 60M). Compared to control or EF treatment, PF further enhanced the enrichment of CD47 on GL-261 cells (FIG. 60N). Collectively, these findings demonstrate that PF hydrogel promotes an immune-stimulating TME in GL-261 brain tumors in mice.

### Example 21

The example demonstrates that aCD47/PF prevents orthotopic GBM recurrence after surgery.

Given that surgical resection is the current standard of care for GBM, a brain tumor resection model was developed to further test the aCD47/PF hydrogel as potential GBM therapy. At day 8 after tumor cell inoculation, the tumor developed to adequate size with small tumor islands around the primary tumor mass evident by MRI (FIG. 61A). Based on findings in a pilot study day 8 was selected to be the optimal time for surgery in the GBM resection model (FIG. 61B i-iii). Resection did not induce neurological impairment or any notable side effects. To validate the model following surgery, mice were immediately sacrificed to confirm the efficiency of resection. The resection cavity could be clearly observed from H&E-stained brain sections (FIG. 61C). The primary tumor mass was almost completely removed, with small tumor deposits remaining in the normal brain parenchyma around the surgical cavity. The extent of tumor resection was quantitatively assessed by bioluminescence analysis, which showed that ˜95% of GBM mass was removed by the surgery (FIG. 61D). These results demonstrate the accurate surgical resection of intracranial GBM in mice, which closely mimics the maximal resection procedure adopted for human GBM patient. Although survival analysis showed that surgical resection extended survival time (median survival, 28.5 days resected vs 22 days untreated), the benefit was modest and all mice succumbed to tumor recurrence (FIG. 61E).

To test the inhibition of tumor recurrence by aCD47/PF hydrogel, aCD47/PF solution was intra-operatively infused into the resection cavity (FIG. 61B iv). In the presence of biological fluid, aCD47/PF gelation was formed rapidly within the resection cavity. This in situ hydrogel seamlessly filled up the cavity left by tumor resection, thereby serving as a reservoir for long-term, localized release of therapeutic agents. Bioluminescence imaging confirmed that all mice developed a well-demarcated tumor at day 8 post-inoculation. The luminescence intensity from brain tumors was significantly reduced after surgical tumor removal (day 9) (FIG. 61F). Similar to the resected mice in the control group, tumor recurrence and growth occurred aggressively in mice that underwent resection followed by EF treatment (FIG. 5f-h). All EF treated mice died within 36 days and showed no difference in survival time, as compared with the mice received tumor resection only (median survival, 29.5 vs 28.5 days) (FIG. 61I). Single drug treatment with PF or aCD47/EF significantly reduced tumor recurrence, with tumor burdens sharply decreased compared to that of untreated resected mice (FIGS. 61F-61H). Both treatments prolonged the median survival time (63 and 39 days, respectively) and improved survival rate of 50% and 25%, respectively (FIG. 61I). Strikingly, no tumor growth was found, by either IVIS or MRI imaging in brain tissues of mice treated with combination aCD47/PF therapy (FIGS. 61F-61H). Resection surgery plus aCD47/PF treatment eliminated tumor recurrence, leading to 100% survival (FIG. 61I), in contrast to aCD47/PF treated mice without resection. Collectively, these data suggest that site-specific aCD47/PF hydrogel implantation as an adjunct therapy to surgical resection shows promise as a new treatment approach for GBM.

### Example 22

This example demonstrates that aCD47/PF elicits durable antitumor immune response.

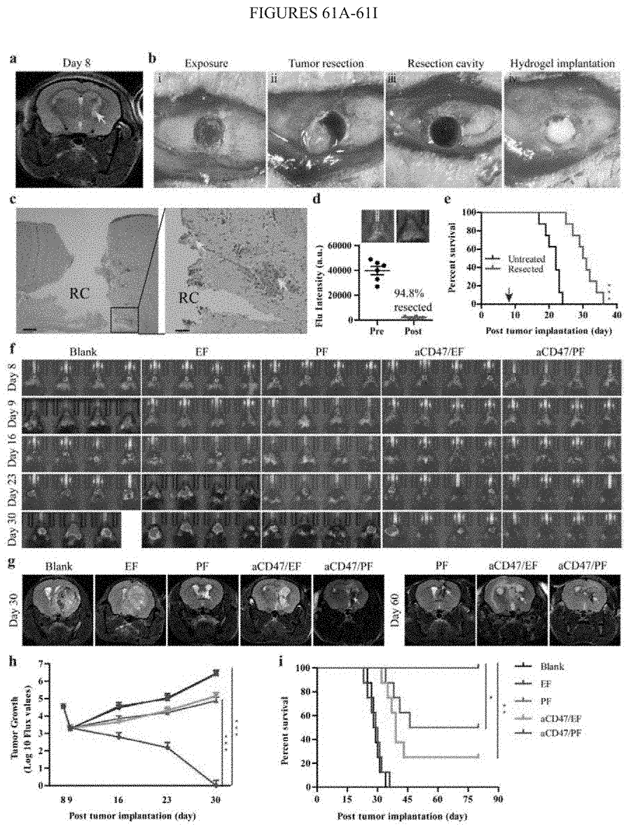

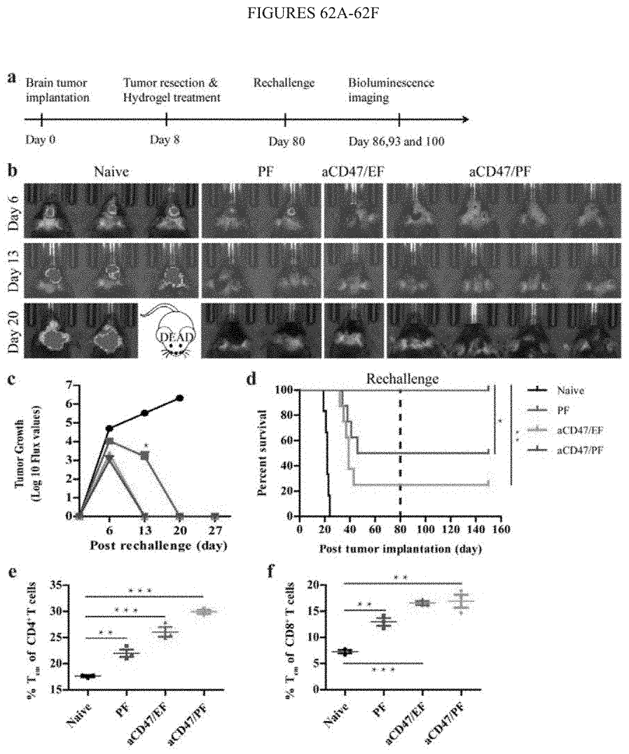

Mice that survived following an initial treatment, called long-term survivors, were rechallenged with GL-261 cells to assess whether localized aCD47/PF elicited a memory immune response (FIG. 62A). As illustrated by the bioluminescence imaging, naïve mice developed progressive and large tumors, which led to 100% fatality within 24 days after tumor cell inoculation (FIGS. 62B-62D). In contrast, although some mice exhibited initial tumor growth in the first few days, no bioluminescence signal was detected in the PF, aCD47/EF, and aCD47/PF groups on day 20 post-rechallenge (FIGS. 62B-62C). These results unravel effective inhibition of tumor growth and a robust memory immune response in the surviving mice. Indeed, in the rechallenged long-term surviving mice, flow cytometry analyses of immune cells in the spleen showed increased percentages of CD4+CD44_{high}CD62_{low} T effector memory cells (CD4+ Tem) and CD8+CD44_{high}CD62L_{low} T effector memory cells (CD8⁺ Tem) (FIGS. 62E-62F). Notably, aCD47/PF-treated mice possessed a much higher survival rate and larger proportions of CD4+ (29.9% vs. 17.6%) and CD8+ Tem (16.9% vs. 7.3%), as compared with Blank group (FIGS. 62D-62F). These results strongly substantiate that a robust and durable antitumor memory immune response was established by a single localized aCD47/PF hydrogel treatment.

Examples 17-22 describe the development of a self-assembling prodrug hydrogel system to deliver both PTX and aCD47 following GBM resection. It was demonstrated that a well-defined aCD47/PF hydrogel was formed rapidly after deposition of aCD47-containing PF solution into the resection cavity. This in situ formed filaments hydrogel seamlessly filled the cavity left by GBM resection, and served as a reservoir for long-term, localized release of both PTX and aCD47 to residual tumor tissue. These in vivo results revealed that PF hydrogel elicited an immune-stimulating TME with enhanced infiltration of TAMs, DCs, and CD8+ T cells and enrichment of CD47 on cancer cells. PTX worked synergistically with the locally released aCD47 to promote phagocytosis of tumor cells by macrophages and also trigger robust T cell mediated antitumor immune response. As such, this chemo-immunotherapy aCD47/PF hydrogel significantly suppressed tumor recurrence following post-surgical removal of GBM, and demonstrated a striking 100% survival rate. Moreover, the local administration of the hydrogel generated robust effector T cell memory, thereby also preventing tumor recurrence. It is noteworthy that this self-supporting hydrogel contains only the PTX prodrug, thus avoiding any potential toxicities from additional excipient materials. More importantly, the in situ formed hydrogel increases the therapeutic concentration at the target site while preventing leakage of the drugs into blood stream and major organs, so as to reduce off-target side effects. Collectively, these results suggest that site-specific aCD47/PF hydrogel injection following surgical resection may be a clinically-relevant chemo-immunotherapy strategy for the treatment of recurrent GBM.

### REFERENCES

All references, including publications, patent applications, and patents, cited herein are hereby incorporated by reference to the same extent as if each reference were individually and specifically indicated to be incorporated by reference and were set forth in its entirety herein.
1. Lesniak, M. S. & Brem, H. Targeted therapy for brain tumours. Nat. Rev. Drug Discov. 3, 499-508 (2004).
2. McGirt, M. J. et al. Gliadel (BCNU) wafer plus concomitant temozolomide therapy after primary resection of glioblastoma multiforme. J. Neurosurg. 110, 583-588 (2009).
3. Attenello, F. J. et al. Use of Gliadel (BCNU) wafer in the surgical treatment of malignant glioma: a 10-year institutional experience. Ann. Surg. Oncol. 15, 2887-2893 (2008).
4. Chowdhary, S. A., Ryken, T. & Newton, H. B. Survival outcomes and safety of carmustine wafers in the treatment of high-grade gliomas: a meta-analysis. J. Neurooncol. 122, 367-382 (2015).
5. Stupp, R. et al. Effects of radiotherapy with concomitant and adjuvant temozolomide versus radiotherapy alone on survival in glioblastoma in a randomised phase III study: 5-year analysis of the EORTC-NCIC trial. Lancet Oncol. 10, 459-466 (2009).
6. Stupp, R. et al. Radiotherapy plus concomitant and adjuvant temozolomide for glioblastoma. N. Engl. J. Med. 352, 987-996 (2005).
7. Fesnak, A. D., June, C. H. & Levine, B. L. Engineered T cells: the promise and challenges of cancer immunotherapy. Nat. Rev. Cancer 16, 566-581 (2016).
8. Ribas, A. & Wolchok, J. D. Cancer immunotherapy using checkpoint blockade.

Science 359, 1350-+(2018).
9. Sampson, J. H., Gunn, M. D., Fecci, P. E. & Ashley, D. M. Brain immunology and immunotherapy in brain tumours. Nat. Rev. Cancer 20, 12-25 (2020).
10. Akhavan, D. et al. CAR T cells for brain tumors: Lessons learned and road ahead. Immunol. Rev. 290, 60-84 (2019).
11. Hambardzumyan, D., Gutmann, D. H. & Kettenmann, H. The role of microglia and macrophages in glioma maintenance and progression. Nat. Neurosci. 19, 20-27 (2016).
12. Badie, B. & Schartner, J. M. Flow cytometric characterization of tumor-associated macrophages in experimental gliomas. Neurosurgery 46, 957-962 (2000).
13. Gholamin, S. et al. Disrupting the CD47-SIRPalpha anti-phagocytic axis by a humanized anti-CD47 antibody is an efficacious treatment for malignant pediatric brain tumors. Sci. Transl. Med. 9 (2017).
14. Gordon, S. R. et al. PD-1 expression by tumour-associated macrophages inhibits phagocytosis and tumour immunity. Nature 545, 495-499 (2017).
15. Hutter, G. et al. Microglia are effector cells of CD47-SIRP alpha antiphagocytic axis disruption against glioblastoma. Proc. Natl. Acad. Sci. USA 116, 997-1006 (2019).
16. Willingham, S. B. et al. The CD47-signal regulatory protein alpha (SIRPa) interaction is a therapeutic target for human solid tumors. Proc. Natl. Acad. Sci. USA 109, 6662-6667 (2012).
17. Jaiswal, S. et al. CD47 is upregulated on circulating hematopoietic stem cells and leukemia cells to avoid phagocytosis. Cell 138, 271-285 (2009).
18. Sick, E. et al. CD47 update: a multifaceted actor in the tumour microenvironment of potential therapeutic interest. Br. J. Pharmacol. 167, 1415-1430 (2012).
19. Barclay, A. N. & van den Berg, T. K. The Interaction Between Signal Regulatory Protein Alpha (SIRP alpha) and CD47: Structure, Function, and Therapeutic Target. Annu. Rev. Immunol. 32, 25-50 (2014).
20. Sockolosky, J. T. et al. Durable antitumor responses to CD47 blockade require adaptive immune stimulation. Proc. Natl. Acad. Sci. USA 113, E2646-2654 (2016).
21. Tseng, D. et al. Anti-CD47 antibody-mediated phagocytosis of cancer by macrophages primes an effective antitumor T-cell response. Proc. Natl. Acad. Sci. USA 110, 11103-11108 (2013).
22. Liu, X. et al. CD47 blockade triggers T cell-mediated destruction of immunogenic tumors. Nat. Med. 21, 1209-1215 (2015).
23. Advani, R. et al. CD47 Blockade by Hu5F9-G4 and Rituximab in Non-Hodgkin's Lymphoma. N. Engl. J. Med. 379, 1711-1721 (2018).
24. Gholamin, S. et al. Irradiation or temozolomide chemotherapy enhances anti-CD47 treatment of glioblastoma. Innate Immun. 26, 130-137 (2020).
25. Chen, Q. et al. In situ sprayed bioresponsive immunotherapeutic gel for post-surgical cancer treatment. Nat. Nanotechnol. 14, 89-97 (2019).
26. Huang, Y., Ma, Y., Gao, P. & Yao, Z. Targeting CD47: the achievements and concerns of current studies on cancer immunotherapy. J. Thorac. Dis. 9, E168-E174 (2017).
27. Petrova, P. S. et al. TTI-621 (SIRPalphaFc): A CD47-Blocking Innate Immune Checkpoint Inhibitor with Broad Antitumor Activity and Minimal Erythrocyte Binding. Clin. Cancer Res. 23, 1068-1079 (2017).
28. Gu, L. & Mooney, D. J. Biomaterials and emerging anticancer therapeutics: engineering the microenvironment. Nat. Rev. Cancer 16, 56-66 (2016).
29. Irvine, D. J. & Dane, E. L. Enhancing cancer immunotherapy with nanomedicine. Nat. Rev. Immunol. 20, 321-334 (2020).
30. Ishihara, J. et al. Matrix-binding checkpoint immunotherapies enhance antitumor efficacy and reduce adverse events. Sci. Transl. Med. 9 (2017).
31. Ishihara, J. et al. Targeted antibody and cytokine cancer immunotherapies through collagen affinity. Sci. Transl. Med. 11 (2019).
32. Milling, L., Zhang, Y. & Irvine, D. J. Delivering safer immunotherapies for cancer. Adv. Drug Deliv. Rev. 114, 79-101 (2017).
33. Wang, H. & Mooney, D. J. Biomaterial-assisted targeted modulation of immune cells in cancer treatment. Nat. Mater. 17, 761-772 (2018).
34. Wang, F., Porter, M., Konstantopoulos, A., Zhang, P. & Cui, H. Preclinical development of drug delivery systems for paclitaxel-based cancer chemotherapy. J. Control. Release 267, 100-118 (2017).
35. De Palma, M. & Lewis, C. E. Macrophage regulation of tumor responses to anticancer therapies. Cancer Cell 23, 277-286 (2013).
36. Zhao, Y. et al. Chemotherapy-Induced Macrophage Infiltration into Tumors Enhances Nanographene-Based Photodynamic Therapy. Cancer Res 77, 6021-6032 (2017).
37. Samanta, D. et al. Chemotherapy induces enrichment of CD47(+)/CD73(+)/PDL1(+) immune evasive triple-negative breast cancer cells. Proc. Natl. Acad. Sci. USA 115, E1239-E1248 (2018).
38. Chakroun, R. W. et al. Fine-Tuning the Linear Release Rate of Paclitaxel-Bearing Supramolecular Filament Hydrogels through Molecular Engineering. ACS Nano 13, 7780-7790
39. Lock, L. L. et al. One-Component Supramolecular Filament Hydrogels as Theranostic Label-Free Magnetic Resonance Imaging Agents. ACS Nano 11, 797-805 (2017).
40. Wang, F. et al. Supramolecular Tubustecan Hydrogel as Chemotherapeutic Carrier to Improve Tumor Penetration and Local Treatment Efficacy. ACS Nano 14, 10083-10094 (2020).
41. Chakroun, R. W. et al. Supramolecular Design of Unsymmetric Reverse Bolaamphiphiles for Cell-Sensitive Hydrogel Degradation and Drug Release. Angew. Chem. Int. Ed. Engl. 59, 4434-4442 (2020).
42. Acar, H. et al. Self-assembling peptide-based building blocks in medical applications. Adv. Drug Deliver. Rev. 110, 65-79 (2017).
43. Kumar, V. A. et al. Drug-Triggered and Cross-Linked Self-Assembling Nanofibrous Hydrogels. J. Am. Chem. Soc. 137, 4823-4830 (2015).
44. Schiapparelli, P. et al. Self-assembling and self-formulating prodrug hydrogelator extends survival in a glioblastoma resection and recurrence model. J. Control. Release 319, 311-321 (2020).
45. Sleep, E. et al. Injectable biomimetic liquid crystalline scaffolds enhance muscle stem cell transplantation. Proc. Natl. Acad. Sci. USA 114, E7919-E7928 (2017).
46. Yan, C. et al. Injectable solid hydrogel: mechanism of shear-thinning and immediate recovery of injectable beta-hairpin peptide hydrogels. Soft Matter. 6, 5143-5156 (2010).
47. O'Leary, L. E. R., Fallas, J. A., Bakota, E. L., Kang, M. K. & Hartgerink, J. D. Multi-hierarchical self-assembly of a collagen mimetic peptide from triple helix to nanofibre and hydrogel. Nat. Chem. 3, 821-828 (2011).
48. Wang, F. et al. Tumour sensitization via the extended intratumoural release of a STING agonist and camptothecin from a self-assembled hydrogel. Nat. Biomed. Eng. (2020).
49. Wang, F. et al. Supramolecular prodrug hydrogelator as an immune booster for checkpoint blocker-based immunotherapy. Sci. Adv. 6, eaaz8985 (2020).
50. Sugahara, K. N. et al. Tissue-penetrating delivery of compounds and nanoparticles into tumors. Cancer Cell 16, 510-520 (2009).
51. McCracken, M. N., Cha, A. C. & Weissman, I. L. Molecular Pathways: Activating T Cells after Cancer Cell Phagocytosis from Blockade of CD47 “Don't Eat Me” Signals. Clin. Cancer Res. 21, 3597-3601 (2015).
52. Song, Q. et al. Tumor Microenvironment Responsive Nanogel for the Combinatorial Antitumor Effect of Chemotherapy and Immunotherapy. Nano Lett. 17, 6366-6375 (2017).
53. Pfannenstiel, L. W., Lam, S. S. K., Emens, L. A., Jaffee, E. M. & Armstrong, T. D. Paclitaxel enhances early dendritic cell maturation and function through TLR4 signaling in mice. Cell Immunol. 263, 79-87 (2010).
54. Su, H., Zhang, W., Wang, H., Wang, F. & Cui, H. Paclitaxel-Promoted Supramolecular Polymerization of Peptide Conjugates. J. Am. Chem. Soc. 141, 11997-12004 (2019).
55. Bianco, J. et al. Novel model of orthotopic U-87 MG glioblastoma resection in athymic nude mice. J. Neurosci. Methods 284, 96-102 (2017).
56. Mathios, D. et al. Anti-PD-1 antitumor immunity is enhanced by local and abrogated by systemic chemotherapy in GBM. Sci. Transl. Med. 8 (2016).

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition comprising:
(a) a prodrug comprising a cytotoxic agent conjugated to a hydrophilic moiety by a linker; and
(b) an immunomodulator one or more immunomodulator.

2. The composition of claim 1, wherein the cytotoxic agent comprises a chemotherapeutic agent.

3. The composition of claim 1, wherein the cytotoxic agent is selected from the group consisting of an alkylating agent, a nitrogen mustard alkylating agent, a nitrosourea alkylating agent, an antimetabolite, a purine analog antimetabolite, a pyrimidine analog antimetabolite, a hormonal antineoplastic, a natural antineoplastic, an antibiotic natural antineoplastic, a vinca alkaloid natural antineoplastic, carboplatin, cisplatin, carmustine (BCNU), methotrexate, fluorouracil (5-FU), gemcitabine, goserelin, leuprolide, tamoxifen, aldesleukin, interleukin-2, docetaxel, etoposide, interferon, paclitaxel, a taxane derivative, tretinoin (ATRA), bleomycin, dactinomycin, daunorubicin, doxorubicin, mitomycin, bumetanide, verteporfrin, vorapaxar, and camptothecin.

4. The composition of 1, wherein the cytotoxic agent is camptothecin or paclitaxel.

5. The composition of claim 1, wherein the immunomodulator is an immune checkpoint regulator.

6. The composition of claim 5, wherein the immune checkpoint regulator is an immune checkpoint inhibitor.

7. The composition of claim 6, wherein the immune checkpoint inhibitor is a monoclonal antibody selected from the group consisting of an anti-CTLA-4 antibody, an anti-B7-H4 antibody, an anti-B7-H1 antibody, an anti-LAG3 antibody, an anti-CD47 antibody, and an anti-PD1 antibody.

8. The composition of claim 7, wherein the immune checkpoint inhibitor is an anti-PD1 antibody selected from the group consisting of pembrolizumab, nivolumab, cemiplimab, avelumab, durvalumab, atezolizumab, spartalizumab (PDR001), camrelizumab (SHR1210), sintilimab (IBI308), tislelizumab (BGB-A317), toripalimab (JS 001), MPDL3280A, MEDI4736, AMP-224, AMP-514, and MSB0010718C.

9. The composition of claim 7, wherein the immune checkpoint inhibitor is an anti-CD47 antibody.

10. The composition of claim 1, wherein the immunomodulator is a small molecule.

11. The composition of claim 10, wherein the small molecule is an agonist of a stimulator of interferon genes (STING) protein.

12. The composition of claim 11, wherein the agonist of a STING protein is selected from the group consisting of cGAMP, cyclic dinucleotides, c-di-AMP, ADU-S100/MIW815, MK-1454, dimeric amidobenzimidazole (diABZI), and 5,6-dimethylxanthenone-4-acetic acid (DMXAA).

13. The composition of claim 1, wherein the hydrophilic moiety is a peptide, polypeptide, or oligo ethyleneoxide (OEG).

14. The composition of claim 1, wherein the hydrophilic moiety is a peptide or polypeptide selected from the group consisting of iRGD, RGD, RGDR (SEQ ID NO: 1), HDK, CEA, TAG-72, CyclinBl, Ep-CAM, Her2/neu, CDK4, fibronectin, p53, and ras.

15. The composition of claim 1, wherein the linker comprises a matrix metalloproteinase-2 (MMP-2)-cleavable peptide and a disulfide linker.

16. The composition of claim 15, wherein the MMP-2=cleavable peptide comprises an amino acid sequence selected from the group consisting of PLGLAG (SEQ ID NO: 4), PLGVR (SEQ ID NO: 5), and GPLGIAGQ (SEQ ID NO: 6).

17. The composition of claim 15, wherein the disulfide linker comprises (4-(pyridin-2-yldisulfanyl)butanoate) (buSS) or 4-pyridyldisulfanyl)ethyl carbonate (etcSS).

18. A hydrogel comprising the composition of claim 1.

19. The hydrogel of claim 18, comprising a compound having a formula selected from the group consisting of:

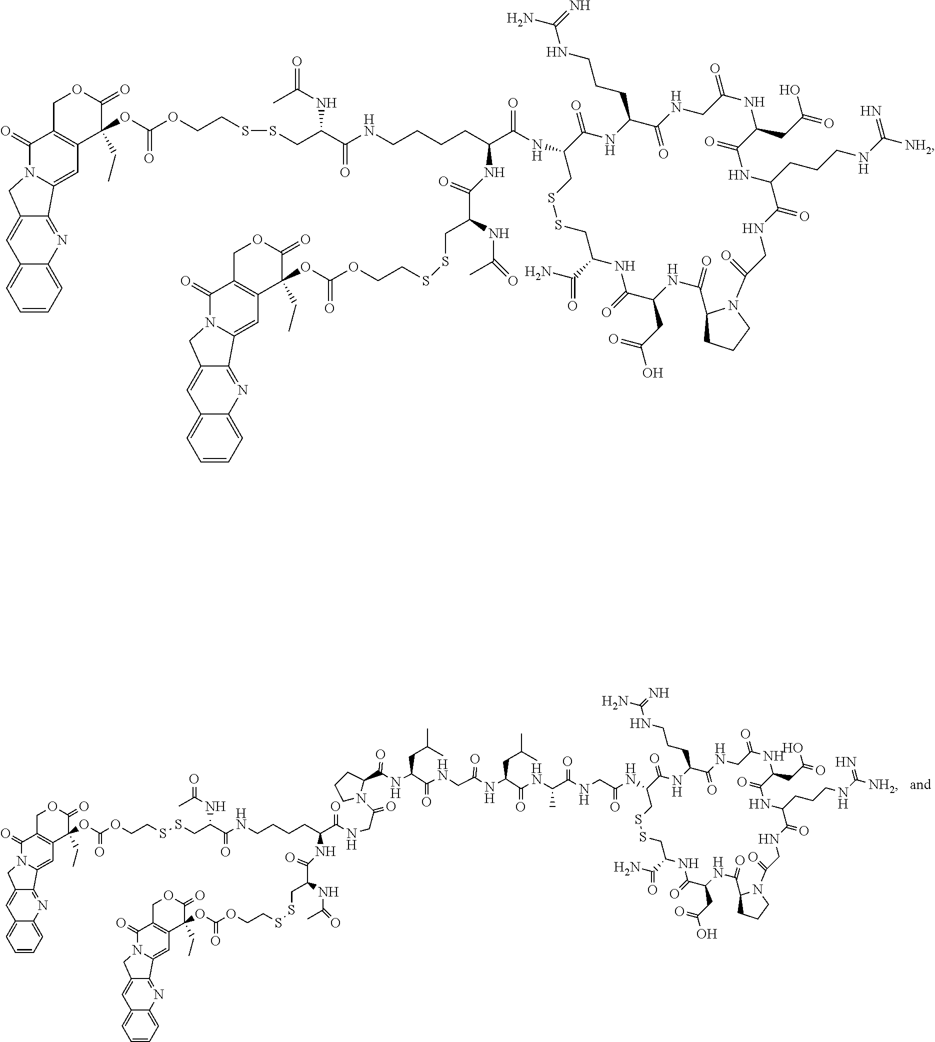

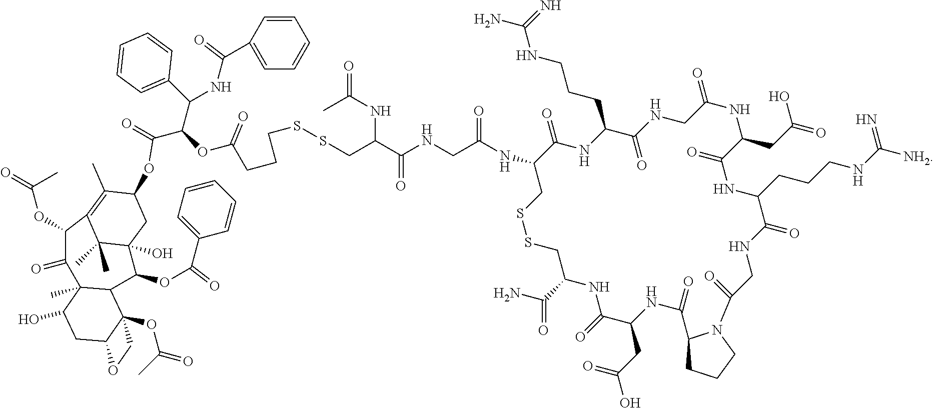

20. A method of killing cancer cells, said method comprising contacting cancer cells with the composition of claim 1, wherein the prodrug spontaneously assembles into a hydrogel and the cytotoxic agent and immunomodulator are released from the composition, thereby killing the cancer cells.

21-27. (canceled)
